(19) United States
(12) Patent Application Publication
(10) Pub. No.: US 2015/0133493 A1
(43) Pub. Date: May 14, 2015

(54) COUMARIN-BASED COMPOUNDS FOR THE TREATMENT OF ALZHEIMER'S DISEASE AND CANCER

(71) Applicant: Sloan-Kettering Institute for Cancer Research, New York, NY (US)

(72) Inventors: Lei Zhu, Schenectady, NY (US); Hakim Djaballah, Scarsdale, NY (US); Yueming Li, New York, NY (US); Christopher Chad Shelton, New York, NY (US)

(21) Appl. No.: 14/323,438

(22) Filed: Jul. 3, 2014

Related U.S. Application Data
(63) Continuation of application No. 13/140,791, filed on Aug. 30, 2011, now abandoned.
(60) Provisional application No. 61/139,830, filed on Dec. 22, 2008.
(60) Provisional application No. 61/255,819, filed on Oct. 28, 2009.

(51) Int. Cl. C07D 493/04; C07D 335/06; C07D 215/20; C07D 311/56

(52) U.S. Cl. CPC C07D 493/04; C07D 311/56; C07D 335/06; C07D 215/20
USPC 514/312; 549/400; 514/457; 549/23; 514/432; 549/384; 546/155

(57) ABSTRACT

Compounds including those of the Formula I

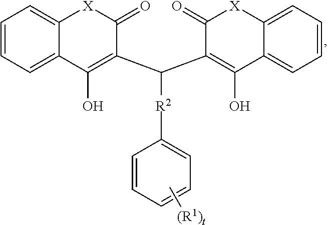

where X, R¹, R² and subscript t are as defined herein, useful as γ-secretase inhibitors, are provided, as are compositions comprising the compounds, as well as methods for use of the compounds for treating or preventing neurodegenerative diseases, such as, for instance, Alzheimer's disease.

### CROSS-REFERENCE TO RELATED APPLICATIONS

This application claims priority to U.S. Provisional Patent Application Ser. No. 61/139,830, filed Dec. 22, 2008 and U.S. Provisional Patent Application Ser. No. 61/255,819, filed Oct. 28, 2009. The entire content of each priority application is incorporated herein by reference.

### FIELD OF THE INVENTION

The invention relates to Coumarin-Based Compounds, pharmaceutical compositions thereof, and methods of treatment of disease therewith.

### BACKGROUND OF THE INVENTION

Alzheimer's disease (AD) is the most prevalent form of dementia. It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of beta-amyloid peptide (Aβ). Aβ is formed from amyloid precursor protein (APP). APP is a ubiquitous membrane-spanning (type 1) glycoprotein, of which three major isoforms (APP695, APP751, and APP770) are known, that undergoes a variety of proteolytic processing events (Selkoe, 1998, Trends Cell Biol. 8:447-453).

Generation of Aβ from APP occurs via separate intracellular proteolytic events involving the enzymes beta-secretase and γ-secretase. Beta-secretase first cleaves APP within the extracellular domain to create soluble APP-beta and beta-CTF (C-terminal fragment), which is then further processed by γ-secretase to release Aβ and γ-CTF. Given that γ-secretase cleaves beta-CTF, beta-CTF has widely been used to monitor γ-secretase activity in cell based and in vitro assays. The cleavage site of APP by γ-secretase appears to be situated within a transmembrane domain, and variability in the site of γ-secretase mediated proteolysis results in Aβ of varying chain lengths comprising heterogeneous C-termini, e.g. Aβ (1-38, “Aβ38”), Aβ (1-40, “Aβ40”) and Aβ (1-42, “Aβ42”). After secretion into the extracellular medium, the initially-soluble Aβ forms aggregate, ultimately resulting in the insoluble deposits and dense neuritic plaques which are the pathological characteristics of AD. Aβ42 is more prone to aggregation than Aβ40 and is the major component of amyloid plaque (Jarrett, et al., 1993, Biochemistry 32:4693-4697; Kuo, et al., 1996, J. Biol. Chem. 271:4077-4081).

Alternatively, APP can be sequentially cleaved by alpha-secretase and γ-secretase to produce soluble APP-alpha, P3 and γ-CTF. Alpha-secretase cleavage precludes the formation of Aβ peptides.

Various interventions in the plaque-forming process have been proposed as therapeutic treatments for AD (see, e.g., Hardy and Selkoe, 2002, Science 297:353-356). One such method of treatment that has been proposed is that of blocking or attenuating the production of Aβ, for example, by inhibition of beta- or γ-secretase. Other proposed methods of treatment include administering a compound(s) which blocks the aggregation of Aβ, or administering an antibody which selectively binds to Aβ. Activation of α-secretase is also an appealing strategy for the development of AD therapy, in that increased alpha-secretase cleavage might lend to lessened Aβ generation.

γ-Secretase is a macromolecular aspartyl protease composed of at least four proteins: presenilin (PS), nicastrin (NCT), PEN-2 and APH-1 (De Strooper, 2003, Neuron 38:9-12). Recently, CD147 and TMP21 have been found to be associated with the γ-secretase complex (Chen, et al., 2006, Nature 440:1208-1212; Zhou et al., 2005, Proc. Natl. Acad. Sci. USA, 102:7499-7504). Among these known components, PS is believed to contain the active site of γ-secretase (Esler et al., 2000, Nat. Cell. Biol., 2:428:434; Li et al., 2000, Nature 405:689-694; Wolfe et al., 1999, Nature 398:513-517). Considerable effort has been made to understand the process of γ-secretase substrate recognition and its catalytic machinery. A PS-dependent protease can process any single-pass transmembrane (TM) protein regardless of its primary sequence as long as the TM protein extracellular domain is smaller than 300 amino acids. Moreover, the size of the extracellular domain appears to determine the efficiency of substrate cleavage (Struhl and Adachi, 2000, Mol. Cell 6:625-636).

The sequential cleavage of APP by two proteases (beta- or alpha-secretase followed γ-secretase) is analogous to a recently defined signaling paradigm, known as regulated intramembrane proteolysis (RIP) (Brown et al., 2000, Cell 100:391-398). RIP generally requires two proteolytic steps to initiate its signaling cascade, whereby the second intramembrane cleavage is dependent on the first cleavage. Indeed, Notch, a type I transmembrane protein employs RIP and is a substrate for γ-secretase cleavage. Activation of Notch (which is γ-secretase dependent) has been implicated in cancer development. As such, inhibitors of γ-secretase activity might not only have implications in the treatment of AD, but may also have benefit in treatment of all diseases in which γ-secretase plays a role.

Cancer also affects a significant number of people. It is currently believed that the Notch signaling pathway is implicated in cancer biology. The Notch signaling pathway involves cell-cell communication, and aberrant Notch signaling has been observed in cancer cells. Such aberrant Notch signaling has been linked to tumor formation. γ-Secretase inhibitors have been found to prevent the generation of the active domain of Notch molecules, thereby suppressing Notch signaling.

There is a need in the art for additional treatments for neurodegenerative diseases and cancer.

### SUMMARY OF THE INVENTION

In one embodiment, the invention provides compounds of the following Formula I

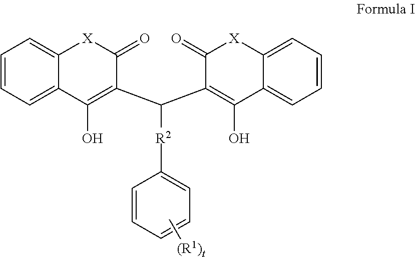

and pharmaceutically acceptable salts thereof, wherein:

each X is independently O, NH, or S;

each R¹ is independently halo, C₁-C₈ alkoxy, cyano, amino, hydroxy, or C₂-C₈ alkyl;

R² is C₁-C₈ alkylene or C₂-C₈ alkenylene; and

t is an integer from 2 to 5.

In another embodiment, the invention provides compounds of the following Formula II

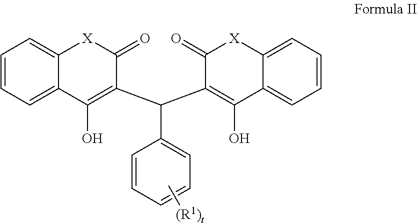

and pharmaceutically acceptable salts thereof, wherein:

each X is independently O, NH, or S;

each R¹ is independently halo, C₁-C₈ alkoxy, cyano, amino, hydroxy, or C₂-C₈ alkyl; and

t is 4 or 5.

In another embodiment, the invention provides compounds of the following Formula III

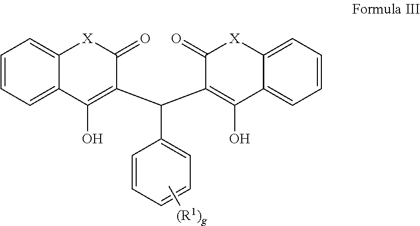

and pharmaceutically acceptable salts thereof, wherein:

each X is independently O, NH, or S;

each R¹ is independently chloro, fluoro, C₂-C₈ alkoxy, cyano, amino, hydroxy, or C₂-C₈ alkyl; and

g is 3.

In another embodiment, the invention provides compounds of the following Formula IV

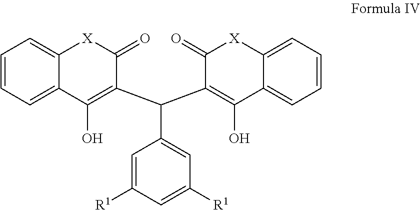

and pharmaceutically acceptable salts thereof, wherein:

each X is independently O or S; and

each R¹ is independently halo, C₁-C₈ alkoxy, cyano, amino, hydroxy, or C₁-C₈ alkyl.

In another embodiment, the invention provides compounds of the following Formula V

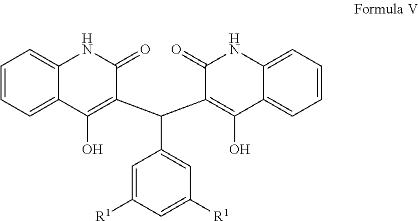

and pharmaceutically acceptable salts thereof, wherein:

each R¹ is independently chloro, bromo, fluoro, iodo, C₁-C₈ alkoxy, cyano, amino, hydroxy, or C₁-C₈ alkyl.

In another embodiment, the invention provides compounds of the following Formula VI

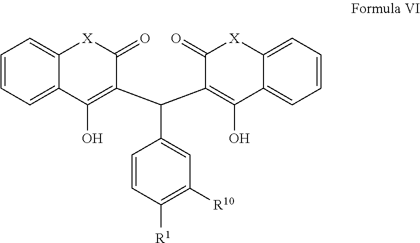

and pharmaceutically acceptable salts thereof, wherein:

each X is independently O, NH or S;

R¹ is C₁-C₈ alkoxy; and

R¹⁰ is halo.

In another embodiment, the invention provides compounds of the following Formula VII

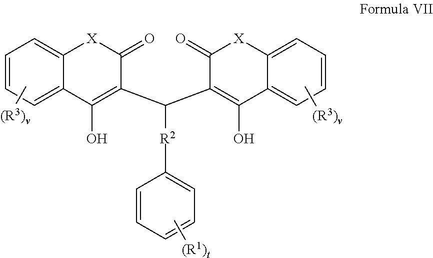

and pharmaceutically acceptable salts thereof, wherein:

each X is independently O, NH, or S;

each R¹ is independently halo, cyano, amino, hydroxy, or C₂-C₈ alkyl;

R² is C₁-C₈ alkylene or C₂-C₈ alkenylene;

each R³ is independently halo or C₁-C₈ alkyl;

t is an integer from 1 to 5; and

each v is independently an integer from 1 to 4.

In another embodiment, the invention provides compounds of the following Formula VIII

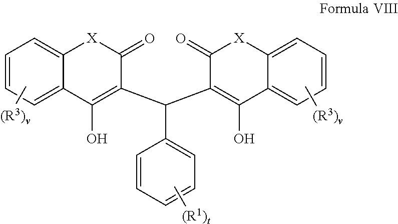

and pharmaceutically acceptable salts thereof, wherein:

each X is independently NH or S;

each R¹ is independently halo, cyano, amino, hydroxy, or C₂-C₈ alkyl;

each R³ is independently halo or C₁-C₈ alkyl;

t is an integer from 1 to 5; and

each v is independently an integer from 1 to 4.

In another embodiment, the invention provides compounds of the following Formula IX

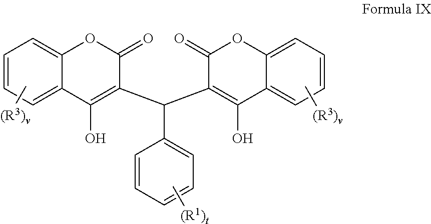

and pharmaceutically acceptable salts thereof, wherein:

each R¹ is independently halo, cyano, amino, hydroxy, or C₂-C₈ alkyl;

each R³ is independently halo or C₁-C₈ alkyl;

t is an integer from 1 to 5; and

each v is independently 3 or 4.

In another embodiment, the invention provides compounds of the following Formula X

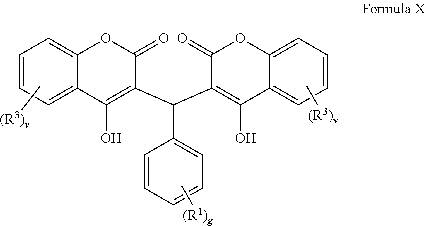

and pharmaceutically acceptable salts thereof, wherein:

each R¹ is independently fluoro, iodo, cyano, or C₂-C₈ alkyl;

each R³ is independently halo or C₁-C₈ alkyl;

g is an integer from 1 to 5; and

each v is independently 1 or 2.

In another embodiment, the invention provides compounds of the following Formula XI

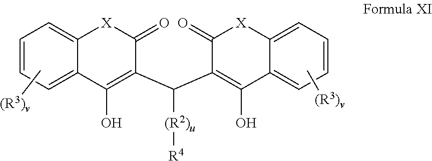

and pharmaceutically acceptable salts thereof, wherein:

each X is independently O or S;

R² is C₁-C₈ alkylene or C₂-C₈ alkenylene;

each R³ is independently halo or C₁-C₈ alkyl;

R⁴ is hydrogen, meta-(trihalomethyl)phenyl, para-ethylphenyl, or para-(C₄-C₈ alkyl)phenyl;

u is 0 or 1; and

each v is independently an integer from 0 to 4. In some embodiments, R⁴ of Formula XI is not hydrogen.

In another embodiment, the invention provides compounds of the following Formula XII

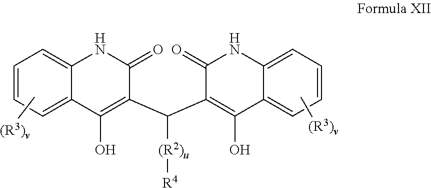

and pharmaceutically acceptable salts thereof, wherein:

R² is C₁-C₈ alkylene or C₂-C₈ alkenylene;

each R³ is independently halo or C₁-C₈ alkyl;

R⁴ is hydrogen, meta-(trihalomethyl)phenyl or para-(C₄-C₈ alkyl)phenyl;

u is 0 or 1; and

each v is independently an integer from 0 to 4. In some embodiments, R⁴ of Formula XII is not hydrogen.

In another embodiment, the invention provides compounds of the following Formula XIII

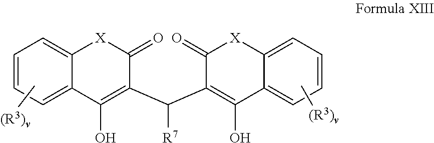

and pharmaceutically acceptable salts thereof, wherein:

each X is independently O, NH, or S;

each R³ is independently halo or C₁-C₈ alkyl;

R⁷ is hydrogen, C₄-C₈ alkenyl or

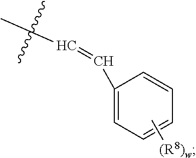

each R⁸ is independently halo, C₁-C₈ alkoxy, cyano, amino, hydroxy, or C₂-C₈ alkyl;

each v is independently an integer from 0 to 4; and

w is an integer from 1 to 5. In some embodiments, R⁷ of formula XIII is not hydrogen.

In another embodiment, the invention provides compounds of the following Formula XIV

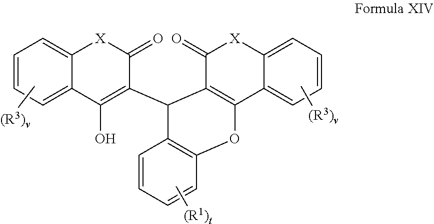

and pharmaceutically acceptable salts thereof, wherein:

each X is independently O, NH, or S;

each R¹ is independently halo, C₁-C₈ alkoxy, cyano, amino, hydroxy, or C₂-C₈ alkyl;

each R³ is independently fluoro, chloro, or C₂-C₈ alkyl;

t is an integer from 0 to 4; and

each v is independently an integer from 1 to 4.

In another embodiment, the invention provides compounds of the following Formula XV

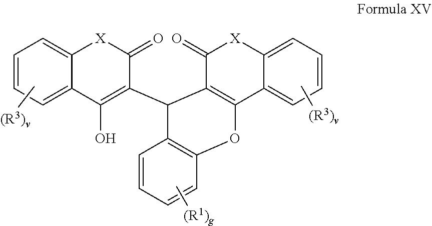

and pharmaceutically acceptable salts thereof, wherein:

each X is independently O, NH, or S;

each R¹ is independently fluoro, C₂-C₈ alkoxy, cyano, amino, or C₂-C₈ alkyl;

each R³ is independently halo or C₁-C₈ alkyl;

g is 1 or 2; and

each v is independently an integer from 0 to 4.

In another embodiment, the invention provides a compound of the following Formula XVI

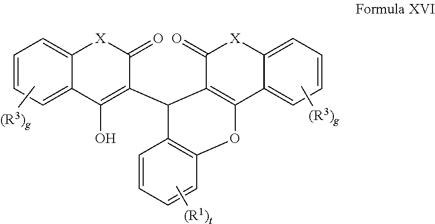

and pharmaceutically acceptable salts thereof, wherein:

each X is independently O, NH, or S;

each R¹ is independently fluoro, bromo, iodo, C₁-C₈ alkoxy, cyano, amino, hydroxy, or C₂-C₈ alkyl;

each R³ is independently halo or C₁-C₈ alkyl;

t is 3 or 4; and

each g is independently an integer from 0 to 4.

In another embodiment, the invention provides compounds of the following Formula XVII

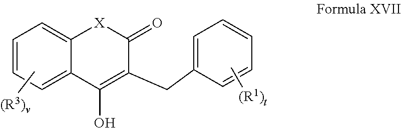

and pharmaceutically acceptable salts thereof, wherein:

X is independently O, NH, or S;

each R¹ is independently halo, cyano, amino, or C₂-C₈ alkyl;

each R³ is independently halo or C₁-C₈ alkyl;

t is an integer from 3 to 5; and

v is an integer from 0 to 4.

In another embodiment, the invention provides compounds of the following Formula XVIII

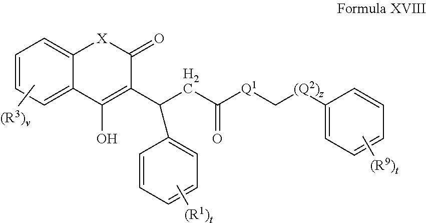

and pharmaceutically acceptable salts thereof, wherein:

X is O, NH, or S;

each R¹ is independently halo, C₁-C₈ alkoxy, cyano, amino, hydroxy, or C₂-C₈ alkyl;

each R³ is independently halo or C₁-C₈ alkyl;

each R⁹ is independently halo, C₁-C₈ alkoxy, cyano, amino, hydroxy, or C₂-C₈ alkyl;

Q¹ is NH or O;

Q² is

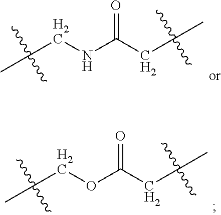 [SMILES: *CNC(=O)C* ; *COC(=O)C*]

each t is independently an integer from 1 to 5;

v is an integer from 0 to 4; and

z is an integer from 0 to 5.

In another embodiment, the invention provides compounds of the following Formula XIX

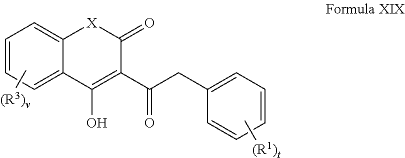

and pharmaceutically acceptable salts thereof, wherein:

X is O, NH, or S;

each R¹ is independently halo, C₁-C₈ alkoxy, cyano, amino, hydroxy, or C₂-C₈ alkyl;

each R³ is independently halo or C₁-C₈ alkyl;

t is an integer from 1 to 5; and

v is an integer from 0 to 4.

In another embodiment, the invention provides compounds of the following Formula XX

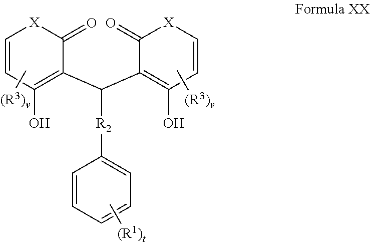

and pharmaceutically acceptable salts thereof, wherein:

each X is independently O or S;

each R¹ is independently halo, C₁-C₈ alkoxy, cyano, amino, hydroxy, or C₂-C₈ alkyl;

R² is C₁-C₈ alkylene or C₂-C₈ alkenylene;

each R³ is independently halo or C₁-C₈ alkyl;

t is an integer from 2 to 5; and

each v is independently an integer from 0 to 2.

In another embodiment, the invention provides compounds of the following Formula XXI

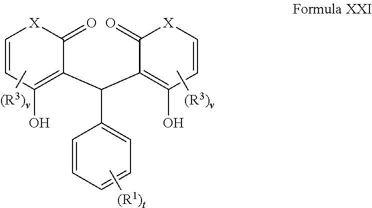

and pharmaceutically acceptable salts thereof, wherein:

each X is independently O or S;

each R¹ is independently halo, C₁-C₈ alkoxy, cyano, amino, hydroxy, or C₂-C₈ alkyl;

each R³ is independently halo or C₂-C₈ alkyl;

t is an integer from 2 to 5; and

each v is independently an integer from 0 to 2.

In another embodiment, the invention provides compounds of the following Formula XXII

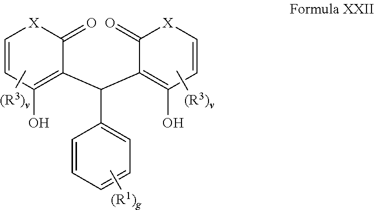

and pharmaceutically acceptable salts thereof, wherein:

each X is independently O or S;

each R¹ is independently halo, C₁-C₈ alkoxy, cyano, amino, hydroxy, or C₁-C₈ alkyl;

each R³ is independently halo or C₂-C₈ alkyl;

g is an integer from 2 to 5; and

each v is independently 0 or 2.

In another embodiment, the invention provides compounds of the following Formula XXIII

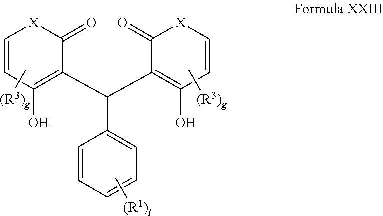

and pharmaceutically acceptable salts thereof, wherein:

each X is independently O or S;

each R¹ is independently halo, C₁-C₈ alkoxy, cyano, amino, hydroxy, or C₂-C₈ alkyl;

each R³ is independently halo or C₁-C₈ alkyl;

t is an integer from 3 to 5; and

each g is 1.

In another embodiment, the invention provides compounds of the following Formula XXIV

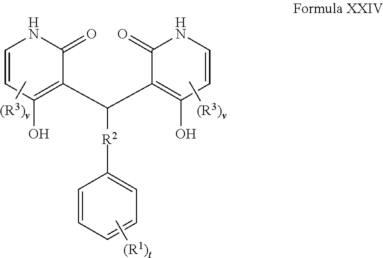

and pharmaceutically acceptable salts thereof, wherein:

each R¹ is independently halo, C₁-C₈ alkoxy, cyano, amino, hydroxy, or C₂-C₈ alkyl;

R² is C₁-C₈ alkylene or C₂-C₈ alkenylene;

each R³ is independently halo or C₁-C₈ alkyl;

t is an integer from 2 to 5; and

each v is independently an integer from 1 to 2.

In another embodiment, the invention provides compounds of the following Formula XXV

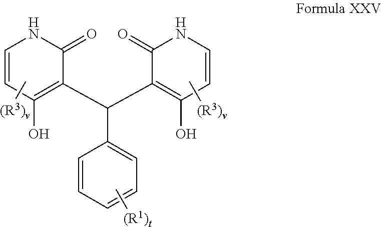

and pharmaceutically acceptable salts thereof, wherein:

each R¹ is independently halo, C₁-C₈ alkoxy, cyano, amino, hydroxy, or C₂-C₈ alkyl;

each R³ is independently halo or C₂-C₈ alkyl;

t is an integer from 2 to 5; and

each v is independently an integer from 1 to 2.

In another embodiment, the invention provides compounds of the following Formula XXVI

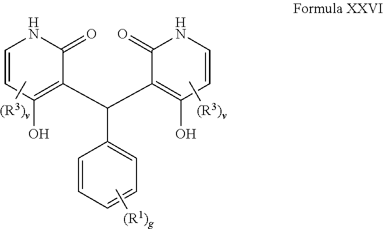

and pharmaceutically acceptable salts thereof, wherein:

each R¹ is independently fluoro, bromo, iodo, cyano, amino, or C₂-C₈ alkyl;

each R³ is independently halo or C₁-C₈ alkyl;

g is an integer from 2 to 5; and

each v is independently an integer from 1 to 2.

In another embodiment, the invention provides compositions comprising an effective amount of a compound of Formula I to XXVI or a pharmaceutically acceptable salt thereof and a pharmaceutically acceptable carrier or vehicle.

In another embodiment, the invention provides methods for treating or preventing cancer, comprising administering to a subject an effective amount of a compound of Formulas I to XXVI, set forth above, or a pharmaceutically acceptable salt thereof.

In another embodiment, the invention provides methods for treating or preventing cancer, comprising administering to a subject an effective amount of a compound of the following Formula A

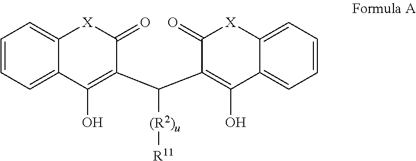

or a pharmaceutically acceptable salt thereof, wherein:

each X is independently O, NH, or S;

R² is C₁-C₈ alkylene or C₂-C₈ alkenylene;

u is 0 or 1; and

R¹¹ is hydrogen;

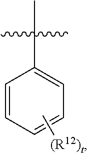

wherein each R¹² is independently fluoro, bromo, iodo, cyano, C₄-C₈ alkoxy, amino, hydroxy, C₁-C₈ alkyl, NHAc, or trihalomethyl and l is 1;

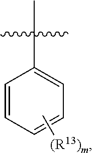

wherein each R¹³ is independently iodo, C₂-C₈ alkoxy, amino, hydroxy, cyano, C₁-C₈ alkyl, NHAc, or trihalomethyl and m is an integer from 2 to 5;

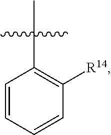

wherein R¹⁴ is bromo, iodo, fluoro, C₃-C₈ alkoxy, amino, hydroxy, cyano, C₁-C₈ alkyl, NHAc, or trihalomethyl;
C₁-C₈ alkyl or C₃-C₈ cycloalkyl; or
C₂-C₈ alkenyl. In some embodiments, R¹¹ of Formula A is not hydrogen.

In another embodiment, the invention provides methods for treating or preventing cancer, comprising administering to a subject an effective amount of a compound of the following Formula B

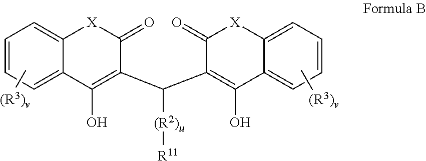

or a pharmaceutically acceptable salt thereof, wherein:

each X is independently O, NH, or S;

R² is C₁-C₈ alkylene or C₂-C₈ alkenylene;

u is 0 or 1;

each R³ is independently halo or C₁-C₈ alkyl;

each v is independently an integer from 1 to 4; and

R¹¹ is hydrogen;

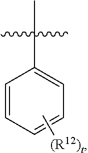

wherein each R¹² is independently bromo, fluoro, iodo, C₄-C₈ alkoxy, amino, C₂-C₈ alkyl, NHAc, or trihalomethyl and l is 1;

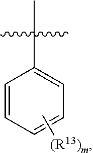

wherein each R¹³ is independently chloro, iodo, fluoro, C₂-C₈ alkoxy, amino, hydroxy, cyano, C₁-C₈ alkyl, NHAc, or trihalomethyl and m is an integer from 2 to 5;
C₁-C₈ alkyl or C₃-C₈ cycloalkyl; or
C₂-C₈ alkenyl. In some embodiments, R¹¹ of formula B is not hydrogen.

In another embodiment, the invention provides methods for treating or preventing cancer, comprising administering to a subject an effective amount of a compound of the following Formula C

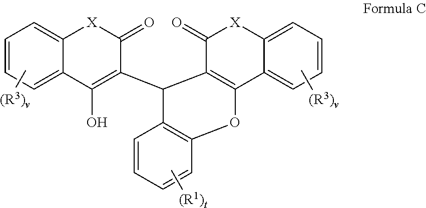

or a pharmaceutically acceptable salt thereof, wherein:

each X is independently O, NH, or S;

each R¹ is independently halo, C₁-C₈ alkoxy, amino, hydroxy, cyano, C₁-C₈ alkyl, NHAc, or trihalomethyl;

each R³ is independently halo or C₁-C₈ alkyl;

t is an integer from 1 to 4; and

each v is independently an integer from 0 to 4.

In another embodiment, the invention provides methods for treating or preventing cancer, comprising administering to a subject an effective amount of a compound of the following Formula D

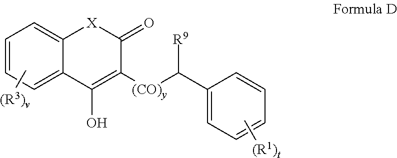

or a pharmaceutically acceptable salt thereof, wherein:

X is O, NH, or S;

each R¹ is independently halo, C₁-C₈ alkoxy, amino, hydroxy, cyano, C₁-C₈ alkyl, NHAc, or trihalomethyl;

each R³ is independently halo or C₁-C₈ alkyl;

R⁹ is hydrogen or

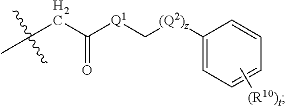

each R¹⁰ is independently halogen, C₁-C₈ alkoxy, cyano, amino, hydroxy, or C₂-C₈ alkyl;

Q¹ is NH or O;

Q² is

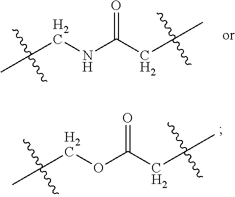 [SMILES: *CNC(=O)C* ; *COC(=O)C*]

each t is independently an integer from 1 to 5;

v is an integer from 0 to 4; and

y is 0 or 1; and

z is an integer from 0 to 5.

In another embodiment, the invention provides methods for treating or preventing cancer, comprising administering to a subject an effective amount of a compound of the following Formula E

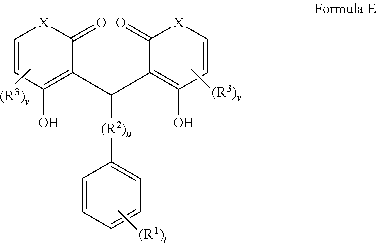

or a pharmaceutically acceptable salt thereof, wherein:

each X is independently O or S;

each R¹ is independently halo, C₁-C₈ alkoxy, amino, hydroxy, cyano, C₁-C₈ alkyl, NHAc, or trihalomethyl;

R² is C₁-C₈ alkylene or C₁-C₈ alkenylene;

each R³ is independently halogen or C₁-C₈ alkyl;

t is an integer from 1 to 5;

each v is independently an integer from 0 to 2; and

u is 0 or 1.

In another embodiment, the invention provides methods for treating or preventing cancer, comprising administering to a subject an effective amount of a compound of the following Formula F

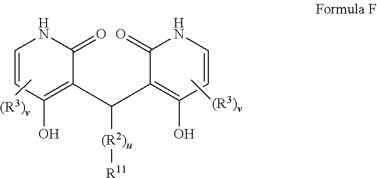

or a pharmaceutically acceptable salt thereof, wherein:

R² is C₁-C₈ alkylene or C₂-C₈ alkenylene;

each R³ is independently halogen or C₁-C₈ alkyl;

each v is independently an integer from 0 to 2;

u is 0 or 1; and

R¹¹ is hydrogen;

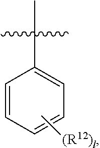

wherein each R¹² is independently halo, C₁-C₈ alkoxy, amino, hydroxy, cyano, C₁-C₈ alkyl, NHAc, or trihalomethyl and l is 1, 2, 4, or 5; or

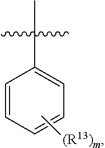

wherein each R¹³ is independently fluoro, chloro, bromo, iodo, C₁-C₈ alkoxy, amino, hydroxy, cyano, C₁-C₈ alkyl, NHAc, or trihalomethyl and m is 3. In some embodiments, R¹¹ of Formula F is not hydrogen.

In another embodiment, the invention provides methods for treating or preventing a neurodegenerative disease, comprising administering to a subject an effective amount of a compound of Formula I to XXVI or A to F, set forth above, or a pharmaceutically acceptable salt thereof.

A compound of Formula I to XXVI, A to F, or a pharmaceutically acceptable salt thereof (a “Coumarin-Based Compound”) is useful for treating or preventing a neurodegenerative disease or cancer (each being a “Condition”).

### BRIEF DESCRIPTION OF THE FIGURES

FIG. 1. This figure provides results of a cell-based assay demonstrating the decrease in Aβ42 (triangles) secretion observed when cells stably transfected with APP were incubated in increasing amounts of compound 37. Secreted amounts of Aβ38 (squares) and Aβ40 (circles) remained relatively constant.

FIG. 2. In vitro characterization of coumarin-dimer allosteric GSIs against various γ-secretase cleavage products. The potency of 7 unique coumarin-based γ-secretase inhibitors were evaluated for efficacy against γ-secretase-mediated production of Aβ40, Aβ42, Aβ38, and Notch. Additionally, the pan-GSI Compound E was also examined in these assays. The IC₅₀ values were calculated from the dose response curves using a non-linear regression analysis in Prism software. IC₅₀ values are presented with standard deviation (n=3 for each data point). The three β-amyloid-detection in vitro assays were modified from our previously reported assay (21) using a biotinylated substrate that eliminated the requirement of anti-β-amyloid biotinylated antibody. Ruthenylated antibodies that detected the -40, -42, or -38 cleavage site were incorporated to detect proteolysis indicative of γ-secretase activity. In vitro Notch assay utilized a recombinant transmembrane portion of the Notch peptide and anti-Notch1 SM320 antibody in conjunction with ruthenylated anti-rabbit secondary antibodies. Electrochemiluminescence was quantified on an Analyzer (BioVeris). The selectivity ratio for Aβ42 inhibition over Aβ40 and Notch are indicated in the two far right columns.

FIG. 3. Cellular evaluation of the coumarin-dimer CS-1 and its selective inhibition of Aβ42. Compounds were incubated with the APPsw-N2A mouse neuroblastoma cells for 24 hours and media were analyzed by biotinylated 4G8 and ruthenylated antibodies specific for each respective cleavage product. (a) CS-1 preferentially abrogates Aβ42 production with no effect on Aβ40 or Aβ38. (b) The GSI Compound E exhibits no inhibitory selectivity for inhibition of β-amyloid peptides. (c) The GSM indomethacin reduces Aβ42 production, potently increases Aβ38, and has little effect on Aβ40. (d) Immunoprecipitation mass spectrometry analysis of CS-1 effect on secreted β-amyloid species. Aβ peptides were immunoprecipitated using 4G8 antibody and isolated with Protein G+/A agarose beads. Samples were analyzed by MALDI-MS. Samples shown are representative and each data point was performed in triplicate. (e) Cell-based Notch cleavage assay. HEK-293 cells were transfected with ΔE Notch construct and then Compound E and CS-1 were evaluated for their ability to inhibit γ-secretase-mediated Notch intracellular domain production. Compound E inhibitor was able to prevent production of NICD, however CS-1 did not affect this cleavage. Western blot is representative and was performed in triplicate. (f) Effect of CS-1 on AICD production. N2A APPsw cell membrane was prepared and incubated with the indicated concentrations of CS-1 at 37° C. for 2 hours. The generated AICD and APP-CTFs were detected by Western Blotting using APPc antibody. Western blot is representative and was performed in triplicate.

FIG. 4. Kinetic analysis of allosteric GSIs and evaluation of their effect on the γ-secretase active site architecture. (a) Kinetic analysis of CS1 was performed using our modified version of a previously reported in vitro γ-secretase activity assay. The inhibition kinetics were analyzed by using a non-linear curve fit with the Michaelis-Menten equation. Upper right inset: we replotted slopes against the inhibitor concentrations after performing double reciprocal conversion. (b) Schematic representation of the allosteric binding of the di-coumarin compounds to γ-secretase. This binding ultimately causes an alteration at the active site of γ-secretase. Black rectangle represents the coumarin-dimer compound. (c) The binding of L458 to the active site of γ-secretase and its interaction at various subpockets within the enzyme. (d) Chemical structure of the four photoaffinity probes utilized in the characterization of CS-1 effect on active site architecture. Hydroxyethylamine and benzophenone moieties are marked by blue and red, respectively. (e) Evaluation of CS-1 effect on the photolabeling of four probes. CS-1 has little to no effect on the ability of JC-8 and L505 to label the active site at the S1′ and S3′ sites, respectively. CS-1 blocks photoincorporation of the benzophenone group of the L646 and GY-4 compounds that label the S2 and S1 subsites, respectively. (f) Evaluation of CS-2 effect on the active site photolabeling by L505 and GY4. (g) Effect of Compound E on active site photolabling. Compound E at 2 μM completely suppressed photolabeling of all four probes. Blotting was performed for PS1-NTF. The photolabeling blots are representative and were performed in triplicate.

FIG. 5. Di-coumarin binding alters the active site of γ-secretase and preferentially alters Aβ42 cleavage. (a) Schematic representation of the AGSI effect on the γ-secretase active site binding pockets. Binding of CS-1 alters the S1 and S2 subsites within the active site of γ-secretase that were probed by GY-4 and L646, respectively, and ultimately leads to a selective inhibition of Aβ42. Active site conformational change is depicted by a change in shape and color at the S2 and S2 subsites. (b) The P2-P3′ residues of Aβ38, Aβ40, Aβ42, and Notch. Alteration of the S2 and S1 subsites may influence Aβ42 production more significantly than other cleavages.

### DETAILED DESCRIPTION OF THE INVENTION

### I. Definitions

The following definitions are used in connection with the Coumarin-Based Compounds:

The term “—C₁-C₈ alkyl,” as used herein unless otherwise defined, refers to a straight chain or branched non-cyclic hydrocarbon having from 1 to 8 carbon atoms, wherein one of the hydrocarbon's hydrogen atoms has been replaced by a single bond. Representative straight chain —C₁-C₈ alkyls include -methyl, -ethyl, -n-propyl, -n-butyl, -n-pentyl, n-heptyl, n-hexyl, and n-octyl. Representative branched —C₁-C₈ alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl.

The term “—C₃-C₈ cycloalkyl,” as used herein unless otherwise defined, refers to a cyclic hydrocarbon having from 3 to 8 carbon atoms, wherein one of the hydrocarbon's hydrogen atoms has been replaced by a single bond. Representative —C3-C8 cycloalkyls include cyclopropyl, cyclobutyl, cyclopentyl, cyclohexyl, cycloheptyl, and cyclooctyl.

The term “halo,” as used herein unless otherwise defined, refers to —F, —Cl, —Br or —I.

The term “subject,” as used herein unless otherwise defined, is a mammal, e.g., a human, mouse, rat, guinea pig, dog, cat, horse, cow, pig, or non-human primate, such as a monkey, chimpanzee, or baboon. In one embodiment, the subject is a human.

The term “pharmaceutically acceptable salt,” as used herein unless otherwise defined, is a salt of an acidic or basic group on the Coumarin-Based Compounds. Illustrative salts of a basic group include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, and pamoate (i.e., 1,1′-methylene-bis-(2-hydroxy-3-naphthoate)) salts. The term “pharmaceutically acceptable salt” also refers to a salt of a Coumarin-Based Compound having an acidic functional group, such as a carboxylic acid, phenolic, or enolic functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

An “effective amount” when used in connection with a Coumarin-Based Compound is an amount that is effective for treating or preventing a Condition.

An “effective amount” when used in connection with another anti-cancer agent is an amount that is effective for treating or preventing cancer alone or in combination with a Coumarin-Based Compound. An “effective amount” when used in connection with another anti-neurodegenerative disease agent is an amount that is effective for treating or preventing a neurodegenerative disease alone or in combination with a Coumarin-Based Compound. “In combination with” includes administration within the same composition and via separate compositions; in the latter instance, the other anti-neurodegenerative disease agent is effective for treating or preventing a neurodegenerative disease during a time when the Coumarin-Based Compound exerts its prophylactic or therapeutic effect, or vice versa, and the other anti-cancer agent is effective for treating or preventing cancer during a time when the Coumarin-Based Compound exerts its prophylactic or therapeutic effect, or vice versa.

As used herein, the term “amyloid precursor protein” (“APP”) refers to an integral membrane protein that is expressed in tissues and concentrated in the synapses of neurons. As used herein, the term APP is meant to encompass all isoforms and forms of APP, both wild-type and synthetic. Exemplary APP isoforms include, but are not limited to, APP695 (SEQ ID NO:1), the 695 amino acid splice variant of APP (see GenBank accession no. Y00264 and Kang, et al., 1987, Nature 325:733-736), APP 751 (SEQ ID NO:2), the 751 amino acid splice variant of APP (see Ponte, et al., 1988, Nature 331:525-527), and APP770 (SEQ ID NO:3), the 770 amino acid splice variant of APP (see Kitaguchi, et al., 1988, Nature 331:530-532). Other isoforms of APP include APP714, L-APP752, L-APP733, L-APP696, L-APP677, APP563 and APP365. Use of the term APP herein is meant to include all isoforms containing mutations found in familial AD and other amyloidosis conditions. For example, these mutations include, but are not limited to, the Swedish double mutation (Lys670Asn, Met671 Leu); the London mutation (Val717Ile); the Indiana mutation (Val717Leu); naturally occurring mutations including Val717Phe, Val717Gly, Ala713Thr, and Ala713Val; the Austrian mutation (Thr714Ile); the Iranian mutation (Thr714Ala); the French mutation (Val715Met); the German mutation (Val715Ala); the Florida mutation (Ile716Val); the Australian mutation (Leu723Pro); the Flemish mutation (Ala692Gly); the Dutch mutation (Glu693Gln); the Arctic mutation (Glu693Gly); the Italian mutation (Glu693Lys); the Iowa mutation (Asp694Asn); and the amyloidosis-Dutch type mutation (Glu693Gln). (All numbering herein is relative to the APP770 form). Use of the term APP herein further includes proteins containing one or more additions, deletions, insertions, or substitutions relative to the isoforms described above, and APP proteins from humans and other species. Unless a specific isoform is specified, APP when used herein generally refers to any and all isoforms of APP, with or without mutations, from any species.

As used herein, the term “amyloid-beta (“Aβ”)” refers to a peptide derived from the proteolytic cleavage of APP. Cleavage of Aβ by beta-secretase generates two APP fragments, referred to herein as “beta-CTF” and “soluble beta-APP.” Beta-CTF is an approximately 100 amino acid fragment, wherein the N-terminus of beta-CTF defines the N-terminus of Aβ. An example of a naturally occurring beta-CTF sequence, i.e., the beta-CTF of APP695, is provided in SEQ ID NO:5. Derivatives of the beta-CTF portion of APP provided in SEQ ID NO:5 are well known in the art (see, e.g., Lichtenthaler, et al., 1997, Biochemistry 36:15396-15403; and Selkoe, 1999, Nature 399:A23-A31). Such derivatives can themselves provide a beta-CTF domain or can serve as a starting point for creating additional derivatives. Examples of naturally occurring derivatives of SEQ ID NO:5 are provided by SEQ ID NOs:12-17. Subsequent γ-secretase cleavage of beta-CTF generates the C-terminus of Aβ. Because γ-secretase cleavage of the beta-CTF fragment occurs over a short stretch of amino acids rather than at a single peptide bond, Aβ ranges in size from, e.g., 39 to 43 peptides. However, Aβ peptides of 40 and 42 amino acids in length (“Aβ40” and “Aβ42,” respectively) predominate.

As used herein, the term “γ-secretase” refers to an enzyme(s) with the ability to cleave at the γ-secretase site of a protein having a γ-secretase cleavage site, e.g., APP. As used herein, γ-secretase includes all recombinant forms, mutations, and other variants of γ-secretase so long as these maintain a functional capability to catalyze the cleavage of molecules or substrates bearing γ-secretase cleavage sites.

As used herein, the term “about” or “approximately,” when used in conjunction with a number, refers to any number within 1, 5 or 10% of the referenced number.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

Concentrations, amounts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### II. Coumarin-Based Compounds of Formulas I to XXVI

In one embodiment, the invention provides compounds of the following Formula I

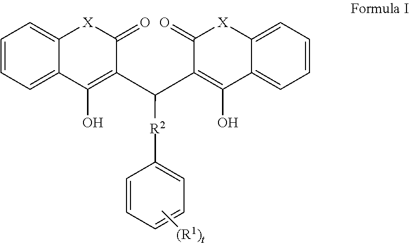

and pharmaceutically acceptable salts thereof, wherein X, R¹, R², and t are as provided above in the summary of the invention for the compounds or pharmaceutically acceptable salts of Formula I.

In some embodiments, X is O. In some embodiments, R¹ is halo. In some embodiments, R² is —CH═CH—. In other embodiments, X is O and R¹ is halo. In other embodiments, X is O and R² is C₂ alkylene. In other embodiments, X is O, R¹ is halo, and R² is C₂ alkylene. In other embodiments, X is O, R¹ is fluoro, and R² is C₂ alkylene.

In other embodiments, the compounds of Formula I have the Formula Ia, set forth below. In some embodiments, the compounds of Formula Ia are those where R^{1a} and R^{1e} are H. In other embodiments, the compounds of Formula Ia are those where R² is —CH═CH—. In some embodiments, R² is trans —CH═CH—. In other embodiments, R² is cis —CH═CH—. In other embodiments, the compounds of Formula Ia are those where R^{1a} and R^{1e} are H and R² is —CH═CH—.

Illustrative examples of the compounds of Formula Ia include those set forth below in Table 1.

TABLE 1
Illustrative examples of the compounds of Formula Ia
Formula Ia

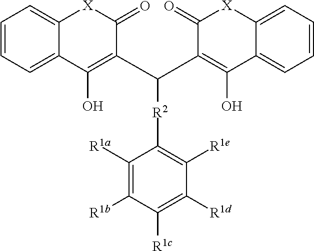

| Cpd. | X | R^{1a} | R^{1b} | R^{1c} | R^{1d} | R^{1e} | R² |
|---|---|---|---|---|---|---|---|
| 1 | O | H | F | F | F | H | HC═CH |
| 2 | O | H | Cl | Cl | Cl | H | HC═CH |
| 3 | O | H | Br | Br | Br | H | HC═CH |
| 4 | O | H | I | I | I | H | HC═CH |
| 5 | NH | H | F | F | F | H | HC═CH |
| 6 | NH | H | Cl | Cl | Cl | H | HC═CH |
| 7 | NH | H | Br | Br | Br | H | HC═CH |
| 8 | NH | H | I | I | I | H | HC═CH |
| 9 | S | H | F | F | F | H | HC═CH |
| 10 | S | H | Cl | Cl | Cl | H | HC═CH |
| 11 | S | H | Br | Br | Br | H | HC═CH |
| 12 | S | H | I | I | I | H | HC═CH |

and pharmaceutically acceptable salts thereof .

In one embodiment, R² of Compound 1-11 or 12 is cis. In another embodiment, R² of Compound 1-11 or 12 is trans.

In another embodiment, the invention provides compounds of the following Formula II

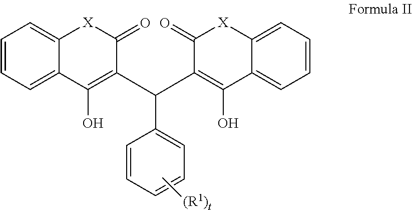

and pharmaceutically acceptable salts thereof, wherein X, R¹, and t are as provided above in the summary of the invention for the compounds or pharmaceutically acceptable salts of Formula II.

In some embodiments, X is O. In some embodiments, R¹ is halo. In other embodiments, X is O, and R¹ is halo. In some embodiments, X is O, and R¹ is fluoro.

In other embodiments, the compounds of Formula II have the Formula IIa, set forth below. In some embodiments, the compounds of Formula IIa are those where R^{1a}, R^{1b}, R^{1c}, R^{1d}, or R^{1e} is halo. In other embodiments, the compounds of Formula IIa are those where R^{1b}, R^{1c}, R^{1d}, and R^{1e} are independently halo.

Illustrative examples of the compounds of Formula IIa include those set forth below in Table 2.

TABLE 2
Illustrative examples of the compounds of Formula IIa
Formula IIa

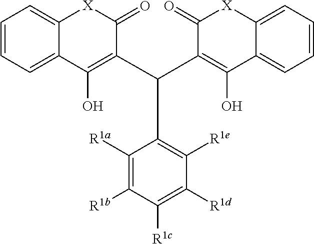

| Cpd. | X | R^{1a} | R^{1b} | R^{1c} | R^{1d} | R^{1e} |
|---|---|---|---|---|---|---|
| 13 | O | H | F | F | F | F |
| 14 | O | F | F | F | F | F |
| 15 | O | H | Cl | Cl | Cl | Cl |
| 16 | O | Cl | Cl | Cl | Cl | Cl |
| 17 | O | H | Br | Br | Br | Br |
| 18 | O | Br | Br | Br | Br | Br |
| 19 | O | H | I | I | I | I |
| 20 | O | I | I | I | I | I |
| 21 | NH | H | F | F | F | F |
| 22 | NH | F | F | F | F | F |
| 23 | NH | H | Cl | Cl | Cl | Cl |
| 24 | NH | Cl | Cl | Cl | Cl | Cl |
| 25 | NH | H | Br | Br | Br | Br |
| 26 | NH | Br | Br | Br | Br | Br |
| 27 | NH | H | I | I | I | I |
| 28 | NH | I | I | I | I | I |
| 29 | S | H | F | F | F | F |
| 30 | S | F | F | F | F | F |
| 31 | S | H | Cl | Cl | Cl | Cl |
| 32 | S | Cl | Cl | Cl | Cl | Cl |
| 33 | S | H | Br | Br | Br | Br |
| 34 | S | Br | Br | Br | Br | Br |
| 35 | S | H | I | I | I | I |
| 36 | S | I | I | I | I | I |

and pharmaceutically acceptable salts thereof.

In another embodiment, the invention provides compounds of the following Formula III

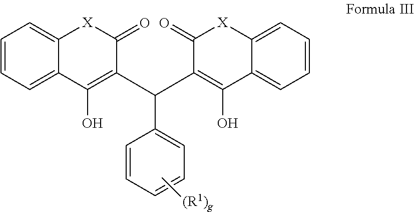

and pharmaceutically acceptable salts thereof, wherein X, R¹, and g are as provided above in the summary of the invention for the compounds or pharmaceutically acceptable salts of Formula III.

In some embodiments, X is O. In some embodiments, R¹ is halo or hydroxy. In other embodiments, X is O and R¹ is halo or hydroxy. In other embodiments, X is O and R¹ is chloro, fluoro, or hydroxy. In other embodiments, X is O, and R¹ is fluoro. In some embodiments, X is NH, and R¹ is fluoro. In other embodiments, X is S, and R¹ is fluoro. In other embodiments, the compounds of Formula III have the Formula IIIa, set forth below. In some embodiments, the compounds of Formula IIIa are those where R^{1a} and R^{1e} are H and R^{1b} through R^{1d} are independently halo. In other embodiments, the compounds of Formula IIIa are those where R^{1a} and R^{1e} are H and R^{1b} through R^{1d} are fluoro.

Illustrative examples of the compounds of Formula IIIa include those set forth below in Table 3.

TABLE 3
Illustrative examples of the compounds of Formula IIIa
Formula IIIa

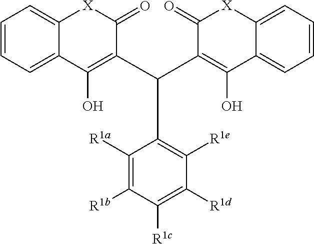

| Cpd. | X | R^{1a} | R^{1b} | R^{1c} | R^{1d} | R^{1e} |
|---|---|---|---|---|---|---|
| 37 | O | H | F | F | F | H |
| 38 | O | H | Cl | Cl | Cl | H |
| 39 | O | H | Br | Br | Br | H |
| 40 | O | H | I | I | I | H |
| 41 | O | H | F | OH | F | H |
| 42 | O | H | F | OH | Cl | H |
| 43 | O | H | F | OH | Br | H |
| 44 | O | H | F | OH | I | H |
| 45 | NH | H | F | F | F | H |
| 46 | NH | H | Cl | Cl | Cl | H |
| 47 | NH | H | Br | Br | Br | H |
| 48 | NH | H | I | I | I | H |
| 49 | NH | H | F | OH | F | H |
| 50 | NH | H | F | OH | Cl | H |
| 51 | NH | H | F | OH | Br | H |
| 52 | NH | H | F | OH | I | H |
| 53 | S | H | F | F | F | H |
| 54 | S | H | Cl | Cl | Cl | H |
| 55 | S | H | Br | Br | Br | H |
| 56 | S | H | I | I | I | H |
| 57 | S | H | F | OH | F | H |
| 58 | S | H | F | OH | Cl | H |
| 59 | S | H | F | OH | Br | H |
| 60 | S | H | F | OH | I | H |

and pharmaceutically acceptable salts thereof.

In another embodiment, the invention provides compounds of the following Formula IV

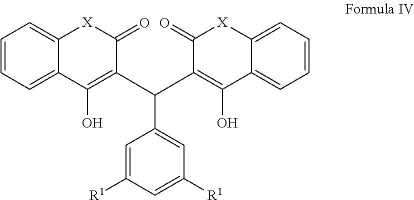

and pharmaceutically acceptable salts thereof, wherein X and R¹ are as provided above in the summary of the invention for the compounds or pharmaceutically acceptable salts of Formula IV.

In some embodiments, X is O. In some embodiments, R¹ is halo. In other embodiments, X is O, and R¹ is halo. In some embodiments, X is O, and R¹ is fluoro.

In other embodiments, the compounds of Formula IV have the Formula IVa, set forth below. In some embodiments, the compounds of Formula IVa are those where R^{1a} or R^{1b} is independently halo. In other embodiments, the compounds of Formula IVa are those where R^{1a} and R^{1b} are independently halo. In other embodiments, the compounds of Formula IVa are those where R^{1a} and R^{1b} are fluoro.

Illustrative examples of the compounds of Formula IVa include those set forth below in Table 4.

TABLE 4
Illustrative examples of the compounds of Formula IVa
Formula IVa

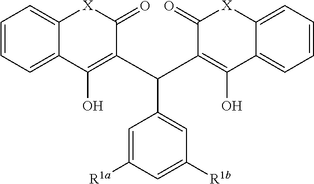

| Cpd. | X | R^{1a} | R^{1b} |
|---|---|---|---|
| 61 | O | F | F |
| 62 | O | Cl | Cl |
| 63 | O | Br | Br |
| 64 | O | I | I |
| 65 | S | F | F |
| 66 | S | Cl | Cl |
| 67 | S | Br | Br |
| 68 | S | I | I |

and pharmaceutically acceptable salts thereof.

In another embodiment, the invention provides compounds of the following Formula V

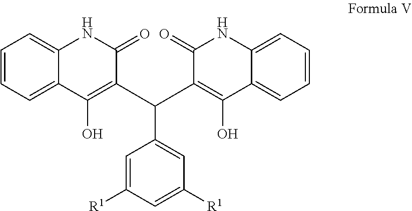

and pharmaceutically acceptable salts thereof, wherein R¹ is as provided above in the summary of the invention for the compounds or pharmaceutically acceptable salts of Formula V.

In some embodiments, R¹ is chloro, bromo, fluoro, iodo, methoxy, cyano, amino, or methyl. In some embodiments, R¹ is chloro, bromo, iodo, methoxy, cyano, amino, or methyl.

In other embodiments, the compounds of Formula V have the Formula Va, set forth below. In some embodiments, the compounds of Formula Va are those where R^{1a} or R^{1b} is independently chloro, bromo, iodo, methoxy, cyano, amino, or methyl. In other embodiments, the compounds of Formula Va are those where R^{1a} and R^{1b} are chloro, bromo, or iodo.

Illustrative examples of the compounds of Formula Va include those set forth below in Table 5.

TABLE 5
Illustrative examples of the compounds of Formula Va
Formula Va

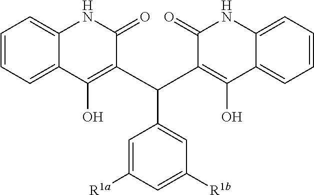

| Cpd. | R^{1a} | R^{1b} |
|---|---|---|
| 69 | Cl | Cl |
| 70 | Br | Br |
| 71 | I | I |
| 72 | OCH₃ | OCH₃ |
| 73 | CN | CN |
| 74 | NH₂ | NH₂ |
| 75 | OH | OH |
| 76 | CH₃ | CH₃ |

and pharmaceutically acceptable salts thereof.

In another embodiment, the invention provides compounds of the following Formula VI

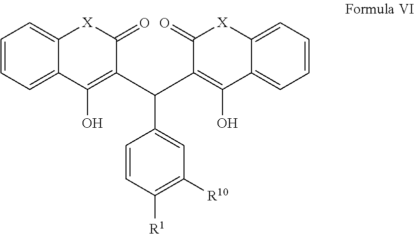

and pharmaceutically acceptable salts thereof, wherein X, R¹, and R¹⁰ are as provided above in the summary of the invention for the compounds or pharmaceutically acceptable salts of Formula VI.

In some embodiments, X is O. In some embodiments, R¹ is methoxy, ethoxy, isopropoxy, or t-butoxy. In other embodiments, X is O, and R¹ is methoxy, ethoxy, isopropoxy, or t-butoxy.

In other embodiments, the compounds of Formula VI have the Formula VIa, set forth below. In some embodiments, the compounds of Formula VIa are those where R^{1a} is methoxy or ethoxy. In other embodiments, the compounds of Formula VIa are those where R^{1a} is methoxy or ethoxy, and R¹⁰ is fluoro.

Illustrative examples of the compounds of Formula VIa include those set forth below in Table 6.

TABLE 6
Illustrative examples of the compounds of Formula VIa
Formula VIa

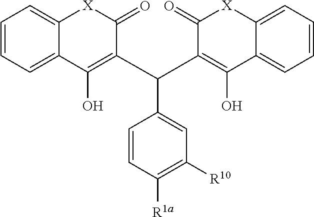

| Cpd. | X | R^{1a} | R¹⁰ |
|---|---|---|---|
| 77 | O | OMe | F |
| 78 | O | OMe | Cl |
| 79 | O | OMe | Br |
| 80 | O | OMe | I |
| 81 | O | OEt | F |
| 82 | O | OEt | Cl |
| 83 | O | OEt | Br |
| 84 | O | OEt | I |
| 85 | NH | OMe | F |
| 86 | NH | OMe | Cl |
| 87 | NH | OMe | Br |
| 88 | NH | OMe | I |
| 89 | NH | OEt | F |
| 90 | NH | OEt | Cl |
| 91 | NH | OEt | Br |
| 92 | NH | OEt | I |
| 93 | S | OMe | F |
| 94 | S | OMe | Cl |
| 95 | S | OMe | Br |
| 96 | S | OMe | I |
| 97 | S | OEt | F |
| 98 | S | OEt | Cl |
| 99 | S | OEt | Br |
| 100 | S | OEt | I |

and pharmaceutically acceptable salts thereof.

In another embodiment, the invention provides compounds of the following Formula VII

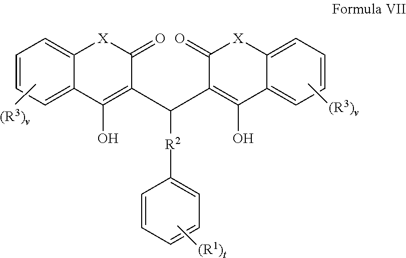

and pharmaceutically acceptable salts thereof, wherein X, R¹, R², R³, t, and v are as provided above in the summary of the invention for the compounds or pharmaceutically acceptable salts of Formula VII.

In some embodiments, X is O. In some embodiments, R¹ is halo. In some embodiments, R² is —CH═CH—. In some embodiments, R³ is fluoro or methyl. In other embodiments, X is O, and R¹ is halo. In other embodiments, X is O, and R3 is fluoro or methyl. In other embodiments, X is O, R¹ is halo, and R³ is fluoro or methyl.

In other embodiments, the compounds of Formula VII have the Formula VIIa, set forth below. In some embodiments, the compounds of Formula VIIa are those where R^{1a} and R^{1e} are H. In other embodiments, the compounds of Formula VIIa are those where R^{1b}, R^{1c}, or R^{1d} is halo. In other embodiments, the compounds of Formula VIIa are those where R^{1b}, R^{1c}, and R^{1d} are independently halo. In other embodiments, the compounds of Formula, VIIa are those where R² is —CH═CH—. In some embodiments, R² is trans —CH═CH—. In other embodiments, R² is cis —CH═CH—. In other embodiments, the compounds of Formula VIIa are those where R^{1a} and R^{1e} are H and R^{1b}, R^{1c}, or R^{1d} is halo. In other embodiments, the compounds of Formula VIIa are those where R^{1a} and R^{1e} are H, R² is —CH═CH— and R^{1b}, R^{1c}, or R^{1d} is halo.

Illustrative examples of the compounds of Formula VIIa include those set forth below in Table 7.

TABLE 7
Illustrative examples of the compounds of Formula VIIa
Formula VIIa

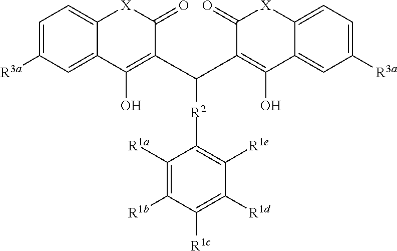

| Cpd. | X | R^{1a} | R^{1b} | R^{1c} | R^{1d} | R^{1e} | R² | R^{3a} |
|---|---|---|---|---|---|---|---|---|
| 101 | O | H | F | F | F | H | HC═CH | CH₃ |
| 102 | O | H | Cl | Cl | Cl | H | HC═CH | CH₃ |
| 103 | O | H | Br | Br | Br | H | HC═CH | CH₃ |
| 104 | O | H | I | I | I | H | HC═CH | CH₃ |
| 105 | O | H | F | F | H | H | HC═CH | CH₃ |
| 106 | O | H | Cl | Cl | H | H | HC═CH | CH₃ |
| 107 | O | H | Br | Br | H | H | HC═CH | CH₃ |
| 108 | O | H | I | I | H | H | HC═CH | CH₃ |
| 109 | O | H | F | F | F | H | HC═CH | F |
| 110 | O | H | Cl | Cl | Cl | H | HC═CH | F |
| 111 | O | H | Br | Br | Br | H | HC═CH | F |
| 112 | O | H | I | I | I | H | HC═CH | F |
| 113 | O | H | F | F | H | H | HC═CH | F |
| 114 | O | H | Cl | Cl | H | H | HC═CH | F |
| 115 | O | H | Br | Br | H | H | HC═CH | F |
| 116 | O | H | I | I | H | H | HC═CH | F |
| 117 | NH | H | F | F | F | H | HC═CH | CH₃ |
| 118 | NH | H | Cl | Cl | Cl | H | HC═CH | CH₃ |
| 119 | NH | H | Br | Br | Br | H | HC═CH | CH₃ |
| 120 | NH | H | I | I | I | H | HC═CH | CH₃ |
| 121 | NH | H | F | F | H | H | HC═CH | CH₃ |
| 122 | NH | H | Cl | Cl | H | H | HC═CH | CH₃ |
| 123 | NH | H | Br | Br | H | H | HC═CH | CH₃ |
| 124 | NH | H | I | I | H | H | HC═CH | CH₃ |
| 125 | NH | H | F | F | F | H | HC═CH | F |
| 126 | NH | H | Cl | Cl | Cl | H | HC═CH | F |
| 127 | NH | H | Br | Br | Br | H | HC═CH | F |
| 128 | NH | H | I | I | I | H | HC═CH | F |
| 129 | NH | H | F | F | H | H | HC═CH | F |
| 130 | NH | H | Cl | Cl | H | H | HC═CH | F |
| 131 | NH | H | Br | Br | H | H | HC═CH | F |
| 132 | NH | H | I | I | H | H | HC═CH | F |
| 133 | S | H | F | F | F | H | HC═CH | CH₃ |
| 134 | S | H | Cl | Cl | Cl | H | HC═CH | CH₃ |
| 135 | S | H | Br | Br | Br | H | HC═CH | CH₃ |
| 136 | S | H | I | I | I | H | HC═CH | CH₃ |
| 137 | S | H | F | F | H | H | HC═CH | CH₃ |
| 138 | S | H | Cl | Cl | H | H | HC═CH | CH₃ |
| 139 | S | H | Br | Br | H | H | HC═CH | CH₃ |
| 140 | S | H | I | I | H | H | HC═CH | CH₃ |
| 141 | S | H | F | F | F | H | HC═CH | F |
| 142 | S | H | Cl | Cl | Cl | H | HC═CH | F |
| 143 | S | H | Br | Br | Br | H | HC═CH | F |
| 144 | S | H | I | I | I | H | HC═CH | F |
| 145 | S | H | F | F | H | H | HC═CH | F |
| 146 | S | H | Cl | Cl | H | H | HC═CH | F |
| 147 | S | H | Br | Br | H | H | HC═CH | F |
| 148 | S | H | I | I | H | H | HC═CH | F |

and pharmaceutically acceptable salts thereof.

In one embodiment, R² of Compound 1-147 or 148 is cis. In another embodiment, R² of Compound 1-147 or 148 is trans.

In another embodiment, the invention provides compounds of the following Formula VIII

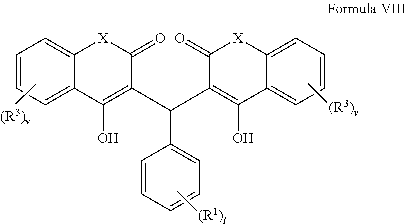

and pharmaceutically acceptable salts thereof, wherein X, R¹, R³, t, and v are as provided above in the summary of the invention for the compounds or pharmaceutically acceptable salts of Formula VIII.

In some embodiments, R¹ is halo. In some embodiments, R³ is fluoro or methyl. In other embodiments, R¹ is halo and R³ is fluoro or methyl.

In other embodiments, the compounds of Formula VIII have the Formula VIIIa, set forth below. In some embodiments, the compounds of Formula VIIIa are those where R^{1a} and R^{1e} are H. In some embodiments, the compounds of Formula VIIIa are those where R^{1b}, R^{1c}, or R^{1d} is independently halo. In some embodiments, the compounds of Formula VIIIa are those where R^{1b}, R^{1c}, and R^{1d} are independently halo. In other embodiments, the compounds of Formula VIIIa are those where R^{1a} and R^{1e} are H, and R^{1b}, R^{1c}, or R^{1d} is independently halo.

Illustrative examples of the compounds of Formula VIIIa include those set forth below in Table 8.

TABLE 8
Illustrative examples of the compounds of Formula VIIIa
Formula VIIIa

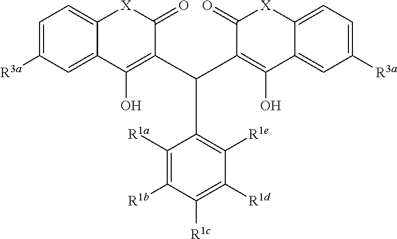

| Cpd | X | R^{1a} | R^{1b} | R^{1c} | R^{1d} | R^{1e} | R^{3a} |
|---|---|---|---|---|---|---|---|
| 149 | NH | H | F | F | F | H | CH₃ |
| 150 | NH | H | Cl | Cl | Cl | H | CH₃ |
| 151 | NH | H | Br | Br | Br | H | CH₃ |
| 152 | NH | H | I | I | I | H | CH₃ |
| 153 | NH | H | F | F | H | H | CH₃ |
| 154 | NH | H | Cl | Cl | H | H | CH₃ |
| 155 | NH | H | Br | Br | H | H | CH₃ |
| 156 | NH | H | I | I | H | H | CH₃ |
| 157 | NH | H | F | F | F | H | F |
| 158 | NH | H | Cl | Cl | Cl | H | F |
| 159 | NH | H | Br | Br | Br | H | F |
| 160 | NH | H | I | I | I | H | F |
| 161 | NH | H | F | F | H | H | F |
| 162 | NH | H | Cl | Cl | H | H | F |
| 163 | NH | H | Br | Br | H | H | F |
| 164 | NH | H | I | I | H | H | F |
| 165 | S | H | F | F | F | H | CH₃ |
| 166 | S | H | Cl | Cl | Cl | H | CH₃ |
| 167 | S | H | Br | Br | Br | H | CH₃ |
| 168 | S | H | I | I | I | H | CH₃ |
| 169 | S | H | F | F | H | H | CH₃ |
| 170 | S | H | Cl | Cl | H | H | CH₃ |
| 171 | S | H | Br | Br | H | H | CH₃ |
| 172 | S | H | I | I | H | H | CH₃ |
| 173 | S | H | F | F | F | H | F |
| 174 | S | H | Cl | Cl | Cl | H | F |
| 175 | S | H | Br | Br | Br | H | F |
| 176 | S | H | I | I | I | H | F |
| 177 | S | H | F | F | H | H | F |
| 178 | S | H | Cl | Cl | H | H | F |
| 179 | S | H | Br | Br | H | H | F |
| 180 | S | H | I | I | H | H | F |

and pharmaceutically acceptable salts thereof.

In another embodiment, the invention provides compounds of the following Formula IX

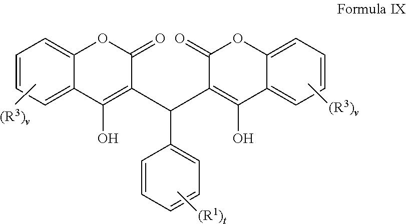

and pharmaceutically acceptable salts thereof, wherein R¹, R³, t, and v are as provided above in the summary of the invention for the compounds or pharmaceutically acceptable salts of Formula IX.

In some embodiments, R¹ is halo. In some embodiments, R³ is fluoro or methyl. In other embodiments, R¹ is halo and R³ is fluoro or methyl.

In other embodiments, the compounds of Formula IX have the Formula IXa, set forth below. In some embodiments, the compounds of Formula IXa are those where R^{1a} and R^{1e} are H. In some embodiments, the compounds of Formula IXa are those where R^{1b}, R^{1c}, or R^{1d} is independently halo. In some embodiments, the compounds of Formula IXa are those where R^{1b}, R^{1c}, and R^{1d} are independently halo. In other embodiments, the compounds of Formula IXa are those where R^{1a} and R^{1e} are H and R^{1b}, R^{1c}, or R^{1d} is independently halo.

Illustrative examples of the compounds of Formula IXa include those set forth below in Table 9.

TABLE 9
Illustrative examples of the compounds of Formula IXa
Formula IXa

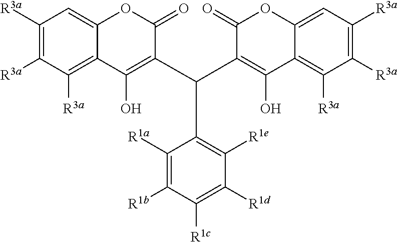

| Cpd. | R^{1a} | R^{1b} | R^{1c} | R^{1d} | R^{1e} | R^{3a} |
|---|---|---|---|---|---|---|
| 181 | H | F | F | F | H | CH₃ |
| 182 | H | Cl | Cl | Cl | H | CH₃ |
| 183 | H | Br | Br | Br | H | CH₃ |
| 184 | H | I | I | I | H | CH₃ |
| 185 | H | F | F | H | H | CH₃ |
| 186 | H | Cl | Cl | H | H | CH₃ |
| 187 | H | Br | Br | H | H | CH₃ |
| 188 | H | I | I | H | H | CH₃ |
| 189 | H | F | F | F | H | F |
| 190 | H | Cl | Cl | Cl | H | F |
| 191 | H | Br | Br | Br | H | F |
| 192 | H | I | I | I | H | F |
| 193 | H | F | F | H | H | F |
| 194 | H | Cl | Cl | H | H | F |
| 195 | H | Br | Br | H | H | F |
| 196 | H | I | I | H | H | F |

and pharmaceutically acceptable salts thereof.

In another embodiment, the invention provides compounds of the following Formula X

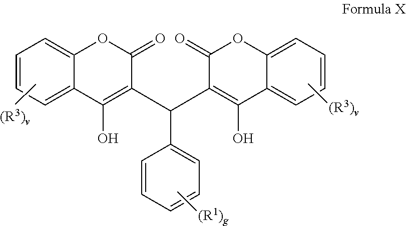

and pharmaceutically acceptable salts thereof, wherein R¹, R³, g, and v are as provided above in the summary of the invention for the compounds or pharmaceutically acceptable salts of Formula X.

In some embodiments, R¹ is fluoro, iodo, cyano, or ethyl. In some embodiments, R³ is fluoro or methyl. In other embodiments, R¹ is fluoro, iodo, cyano, or ethyl and R³ is fluoro or methyl.

In other embodiments, the compounds of Formula X have the Formula Xa, set forth below. In some embodiments, the compounds of Formula Xa are those where R^{1a}, R^{1e}, and R^{3a} are H. In some embodiments, the compounds of Formula Xa are those where R^{1b}, R^{1c}, or R^{1d} is fluoro or iodo. In some embodiments, the compounds of Formula Xa are those where R^{1b}, R^{1c}, and R^{1d} are fluoro. In other embodiments, the compounds of Formula Xa are those where R^{1a}, R^{1e}, and R^{3a} are H and R^{1b}, R^{1c}, or R^{1d} is fluoro or iodo.

Illustrative examples of the compounds of Formula Xa include those set forth below in Table 10.

TABLE 10
Illustrative examples of the compounds of Formula Xa
Formula Xa

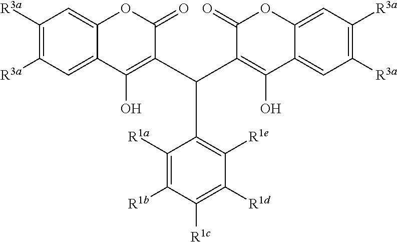

| Cpd. | R^{1a} | R^{1b} | R^{1c} | R^{1d} | R^{1e} | R^{3a} | R^{3b} |
|---|---|---|---|---|---|---|---|
| 197 | H | F | F | F | H | H | CH₃ |
| 198 | H | I | I | I | H | H | CH₃ |
| 199 | H | H | F | H | H | H | CH₃ |
| 200 | H | H | I | H | H | H | CH₃ |
| 201 | H | H | CN | H | H | H | CH₃ |
| 202 | H | H | Et | H | H | H | CH₃ |
| 203 | H | F | F | F | H | H | F |
| 204 | H | I | I | I | H | H | F |
| 205 | H | H | F | H | H | H | F |
| 206 | H | H | I | H | H | H | F |
| 207 | H | H | CN | H | H | H | F |
| 208 | H | H | Et | H | H | H | F |

and pharmaceutically acceptable salts thereof.

In another embodiment, the invention provides compounds of the following Formula XI

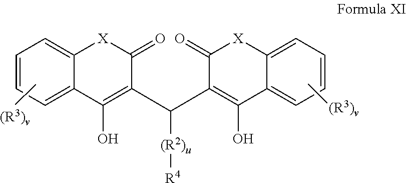

and pharmaceutically acceptable salts thereof, wherein X, R², R³, R⁴, u, and v are as provided above in the summary of the invention for the compounds or pharmaceutically acceptable salts of Formula XI.

In some embodiments, R⁴ is meta-(trihalomethyl)phenyl or para-ethylphenyl. In some embodiments, R² is —CH═CH—. In some embodiments, R³ is fluoro or methyl. In other embodiments, R⁴ is meta-(trihalomethyl)phenyl or para-ethylphenyl and R² is —CH═CH—. In other embodiments, R⁴ is meta-(trihalomethyl)phenyl or para-ethylphenyl and R³ is fluoro or methyl. In other embodiments, R⁴ is meta-(trihalomethyl)phenyl or para-ethylphenyl, R² is —CH═CH—, and R³ is fluoro or methyl.

In other embodiments, the compounds of Formula XI have the Formula XIa, set forth below. In some embodiments, the compounds of Formula XIa are those where R⁴ is meta-(trihalomethyl)phenyl or para-ethylphenyl. In other embodiments, the compounds of Formula XIa are those where R^{3a} is H, fluoro, or methyl. In some embodiments, the compounds of Formula XIa are those where R² is —CH═CH—. In some embodiments, R² is trans —CH═CH—. In other embodiments, R² is cis —CH═CH—. In other embodiments, the compounds of Formula XIa are those where R⁴ is meta-(trihalomethyl)phenyl or para-ethylphenyl and R² is —CH═CH—. In other embodiments, the compounds of Formula XIa are those where R⁴ is meta-(trihalomethyl)phenyl or para-ethylphenyl and R^{3a} is H, fluoro, or methyl. In other embodiments, the compounds of Formula XIa are those where R⁴ is meta-(trihalomethyl)phenyl or para-ethylphenyl, R² is —CH═CH—, and R^{3a} is H, fluoro, or methyl.

Illustrative examples of the compounds of Formula XIa include those set forth below in Table 11.

TABLE 11
Illustrative examples of the compounds of Formula XIa

| | |
|---|---|
| | Formula XIa |
| 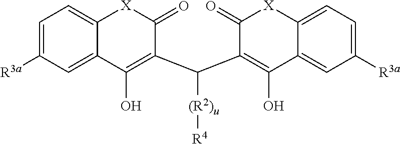 | |

| Cpd. | X | R⁴ | R^{3a} | u | R² |
|---|---|---|---|---|---|
| 209 | O | m-CF₃—C₆H₄ | H | 0 | absent |
| 210 | O | p-C₂H₅—C₆H₄ | H | 0 | absent |
| 211 | O | p-C₃H₇—C₆H₄ | H | 0 | absent |
| 212 | O | m-CF₃—C₆H₄ | CH₃ | 0 | absent |
| 213 | O | p-C₂H₅—C₆H₄ | CH₃ | 0 | absent |
| 214 | O | p-C₃H₇—C₆H₄ | CH₃ | 0 | absent |
| 215 | O | m-CF₃—C₆H₄ | F | 0 | absent |
| 216 | O | p-C₂H₅—C₆H₄ | F | 0 | absent |
| 217 | O | p-C₃H₇—C₆H₄ | F | 0 | absent |
| 218 | O | m-CF₃—C₆H₄ | H | 1 | CH═CH |
| 219 | O | p-C₂H₅—C₆H₄ | H | 1 | CH═CH |
| 220 | O | p-C₃H₇—C₆H₄ | H | 1 | CH═CH |
| 221 | O | m-CF₃—C₆H₄ | CH₃ | 1 | CH═CH |
| 222 | O | p-C₂H₅—C₆H₄ | CH₃ | 1 | CH═CH |
| 223 | O | p-C₃H₇—C₆H₄ | CH₃ | 1 | CH═CH |
| 224 | O | m-CF₃—C₆H₄ | F | 1 | CH═CH |
| 225 | O | p-C₂H₅—C₆H₄ | F | 1 | CH═CH |
| 226 | O | p-C₃H₇—C₆H₄ | F | 1 | CH═CH |
| 227 | S | m-CF₃—C₆H₄ | H | 0 | absent |
| 228 | S | p-C₂H₅—C₆H₄ | H | 0 | absent |
| 229 | S | p-C₃H₇—C₆H₄ | H | 0 | absent |
| 230 | S | m-CF₃—C₆H₄ | CH₃ | 0 | absent |
| 231 | S | p-C₂H₅—C₆H₄ | CH₃ | 0 | absent |
| 232 | S | p-C₃H₇—C₆H₄ | CH₃ | 0 | absent |
| 233 | S | m-CF₃—C₆H₄ | F | 0 | absent |
| 234 | S | p-C₂H₅—C₆H₄ | F | 0 | absent |
| 235 | S | p-C₃H₇—C₆H₄ | F | 0 | absent |
| 236 | S | m-CF₃—C₆H₄ | H | 1 | CH═CH |
| 237 | S | p-C₂H₅—C₆H₄ | H | 1 | CH═CH |
| 238 | S | p-C₃H₇—C₆H₄ | H | 1 | CH═CH |
| 239 | S | m-CF₃—C₆H₄ | CH₃ | 1 | CH═CH |
| 240 | S | p-C₂H₅—C₆H₄ | CH₃ | 1 | CH═CH |
| 241 | S | p-C₃H₇—C₆H₄ | CH₃ | 1 | CH═CH |
| 242 | S | m-CF₃—C₆H₄ | F | 1 | CH═CH |
| 243 | S | p-C₂H₅—C₆H₄ | F | 1 | CH═CH |
| 244 | S | p-C₃H₇—C₆H₄ | F | 1 | CH═CH |

and pharmaceutically acceptable salts thereof.

In one embodiment, R² of compound 218-226, 236-243, or 244 is cis. In another embodiment, R² of compound 218-226, 236-243, or 244 is trans.

In another embodiment, the invention provides compounds of the following Formula XII

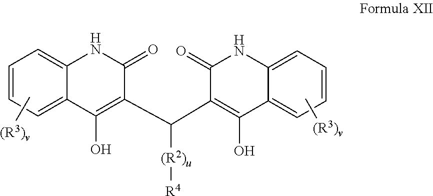

and pharmaceutically acceptable salts thereof, wherein R², R³, R⁴, u and v are as provided above in the summary of the invention for the compounds or pharmaceutically acceptable salts of Formula XII.

In some embodiments, R⁴ is meta-(trihalomethyl)phenyl or para-butylphenyl. In some embodiments, R² is —CH═CH—. In some embodiments, R³ is fluoro or methyl. In other embodiments, R⁴ is meta-(trihalomethyl)phenyl or para-butylphenyl and R² is —CH═CH—. In other embodiments, R⁴ is meta-(trihalomethyl)phenyl or para-butylphenyl and R³ is fluoro or methyl. In other embodiments, R⁴ is meta-(trihalomethyl)phenyl or para-butylphenyl, R² is —CH═CH—, and R³ is fluoro or methyl.

In other embodiments, the compounds of Formula XII have the Formula XIIa, set forth below. In some embodiments, the compounds of Formula XIIa are those where R⁴ is meta-(trihalomethyl)phenyl or para-butylphenyl. In other embodiments, the compounds of Formula XIIa are those where R^{3a} is H, fluoro, or methyl. In some embodiments, the compounds of Formula XIIa are those where R² is —CH═CH—. In some embodiments, R² is trans —CH═CH—. In other embodiments, R² is cis —CH═CH—. In other embodiments, the compounds of Formula XIIa are those where R⁴ is meta-(trihalomethyl)phenyl or para-butylphenyl, R^{1a} is H, fluoro, or methyl, and R² is —CH═CH—.

Illustrative examples of the compounds of Formula XIIa include those set forth below in Table 12.

TABLE 12
Illustrative examples of the compounds of Formula XIIa

| | | | | Formula XIIa |
|---|---|---|---|---|
| 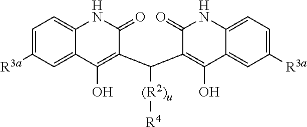 | | | | |
| Cpd. | R⁴ | R^{3a} | u | R² |
| 245 | m-CF₃—C₆H₄ | H | 0 | absent |
| 246 | p-C₄H₉—C₆H₄ | H | 0 | absent |
| 247 | m-CF₃—C₆H₄ | CH₃ | 0 | absent |
| 248 | p-C₄H₉—C₆H₄ | CH₃ | 0 | absent |
| 249 | m-CF₃—C₆H₄ | F | 0 | absent |
| 250 | p-C₄H₉—C₆H₄ | F | 0 | absent |
| 251 | m-CF₃—C₆H₄ | H | 1 | CH═CH |
| 252 | p-C₄H₉—C₆H₄ | H | 1 | CH═CH |
| 253 | m-CF₃—C₆H₄ | CH₃ | 1 | CH═CH |
| 254 | p-C₄H₉—C₆H₄ | CH₃ | 1 | CH═CH |
| 255 | m-CF₃—C₆H₄ | F | 1 | CH═CH |
| 256 | p-C₄H₉—C₆H₄ | F | 1 | CH═CH |

and pharmaceutically acceptable salts thereof.

In one embodiment, R² of Compound 251-255 or 256 is cis. In another embodiment, R² of Compound 251-255 or 256 is trans.

In another embodiment, the invention provides compounds of the following Formula XIII

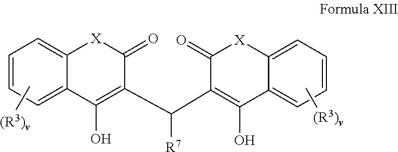

and pharmaceutically acceptable salts thereof, wherein X, R³, R⁷, R⁸ and v are as provided above in the summary of the invention for the compounds or pharmaceutically acceptable salts of Formula XIII.

In certain embodiments of a compound of Formula XIII, R⁷ is

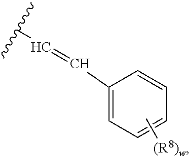

where each R⁸ is independently halo, C₁-C₈ alkoxy, cyano, amino, hydroxy, or C₂-C₈ alkyl and w is an integer from 1 to 5.

In some embodiments, X is O. In some embodiments, R⁸ is methoxy, fluoro, hydroxy, or ethyl. In some embodiments, R³ is fluoro or methyl. In other embodiments, X is O and R⁸ is methoxy, fluoro, hydroxy, or ethyl. In other embodiments, X is O and R³ is fluoro or methyl. In other embodiments, X is O, R³ is fluoro or methyl, and R⁸ is methoxy, fluoro, hydroxy, or ethyl. In other embodiments, X is O; R⁸ is methoxy, fluoro, hydroxy, or ethyl; and v is 0.

In other embodiments, the compounds of Formula XIII have the Formula XIIIa, set forth below. In some embodiments, the compounds of Formula XIIIa are those where R^{3a} is H, fluoro, or methyl. In some embodiments, the compounds of Formula XIIIa are those where R^{7a} is —CH═CH—CH═CH—CH₃ or

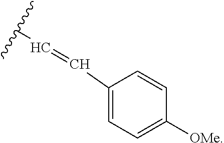 [SMILES: */C=C/c1ccc(OC)cc1]

In one embodiment, the

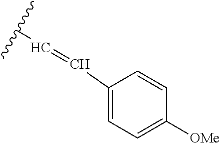 [SMILES: */C=C/c1ccc(OC)cc1]

group is cis. In another embodiment, the

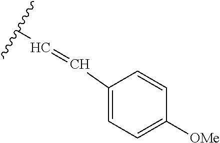 [SMILES: */C=C/c1ccc(OC)cc1]

group is trans. In another embodiment, the —CH═CH—CH═CH—CH₃ group is cis, cis

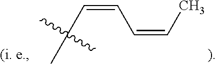 [SMILES: */C=C\C=C/C]

In another embodiment, the —CH═CH—CH═CH—CH₃ group is trans, trans

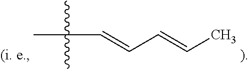 [SMILES: */C=C/C=C/C]

In another embodiment, the —CH═CH—CH═CH—CH₃ group is trans, cis

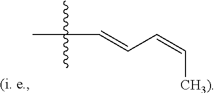

In another embodiment, the —CH═CH—CH═CH—CH₃ group is cis, trans

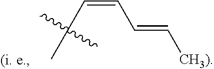

In other embodiments, R^{3a} is H, fluoro, or methyl and R^{7a} is —CH═CH—CH═CH—CH₃ or

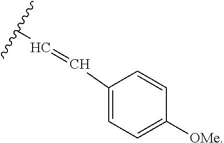 [SMILES: */C=C/c1ccc(OC)cc1]

Illustrative examples of the compounds of Formula XIIIa include those set forth below in Table 13.

TABLE 13
Illustrative examples of the compounds of Formula XIIIa

| | |
|---|---|
| | Formula XIIIa |
| 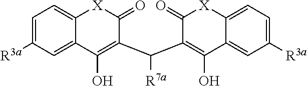 | |

| Cpd. | X | R^{3a} | R^{7a} |
|---|---|---|---|
| 257 | O | H | 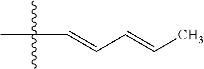 [SMILES: */C=C/C=C/C] |
| 258 | O | H | 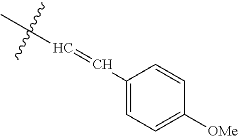 [SMILES: *C=Cc1ccc(OC)cc1] |
| 259 | O | H | 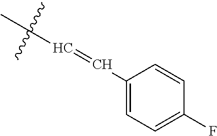 [SMILES: *C=Cc1ccc(F)cc1] |
| 260 | O | H | 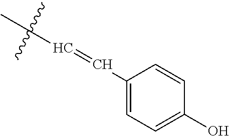 [SMILES: *C=Cc1ccc(O)cc1] |
| 261 | O | H | 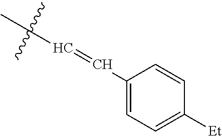 [SMILES: *C=Cc1ccc(CC)cc1] |
| 262 | O | F | 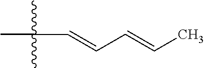 [SMILES: */C=C/C=C/C] |
| 263 | O | F | 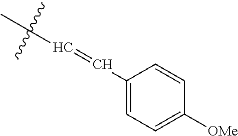 [SMILES: *C=Cc1ccc(OC)cc1] |
| 264 | O | F | 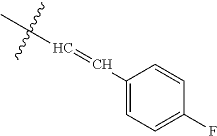 [SMILES: *C=Cc1ccc(F)cc1] |
| 265 | O | F | 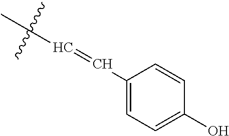 [SMILES: *C=Cc1ccc(O)cc1] |
| 266 | O | F | 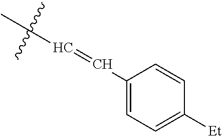 [SMILES: *C=Cc1ccc(CC)cc1] |
| 267 | O | CH₃ | 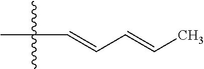 [SMILES: */C=C/C=C/C] |
| 268 | O | CH₃ | 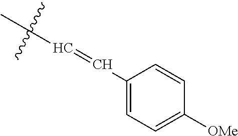 [SMILES: *C=Cc1ccc(OC)cc1] |
| 269 | O | CH₃ | 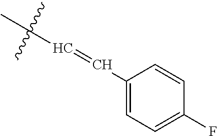 [SMILES: *C=Cc1ccc(F)cc1] |
| 270 | O | CH₃ | 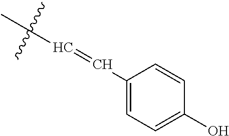 [SMILES: *C=Cc1ccc(O)cc1] |
| 271 | O | CH₃ | 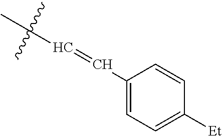 [SMILES: *C=Cc1ccc(CC)cc1] |
| 272 | NH | H | 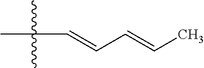 [SMILES: */C=C/C=C/C] |
| 273 | NH | H | 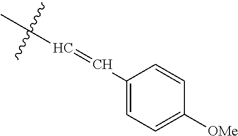 [SMILES: *C=Cc1ccc(OC)cc1] |
| 274 | NH | H | 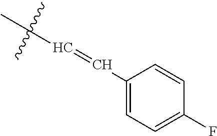 [SMILES: *C=Cc1ccc(F)cc1] |
| 275 | NH | H | 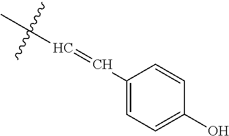 [SMILES: *C=Cc1ccc(O)cc1] |
| 276 | NH | H | 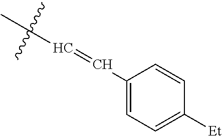 [SMILES: *C=Cc1ccc(CC)cc1] |
| 277 | NH | F | 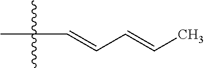 [SMILES: */C=C/C=C/C] |
| 278 | NH | F | 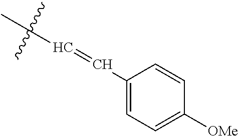 [SMILES: *C=Cc1ccc(OC)cc1] |
| 279 | NH | F | 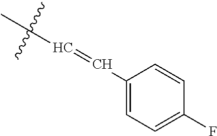 [SMILES: *C=Cc1ccc(F)cc1] |
| 280 | NH | F | 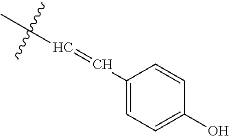 [SMILES: *C=Cc1ccc(O)cc1] |
| 281 | NH | F | 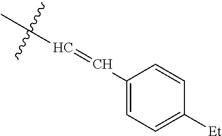 [SMILES: *C=Cc1ccc(CC)cc1] |
| 282 | NH | CH₃ | 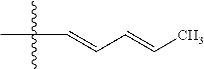 [SMILES: */C=C/C=C/C] |
| 283 | NH | CH₃ | 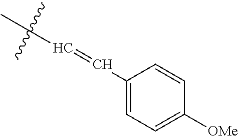 [SMILES: *C=Cc1ccc(OC)cc1] |
| 284 | NH | CH₃ | 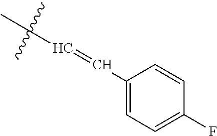 [SMILES: *C=Cc1ccc(F)cc1] |
| 285 | NH | CH₃ | 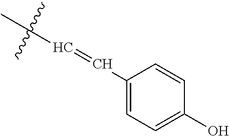 [SMILES: *C=Cc1ccc(O)cc1] |
| 286 | NH | CH₃ | 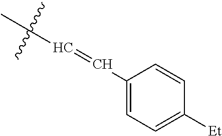 [SMILES: *C=Cc1ccc(CC)cc1] |
| 287 | S | H | 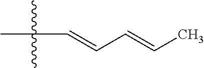 [SMILES: */C=C/C=C/C] |
| 288 | S | H | 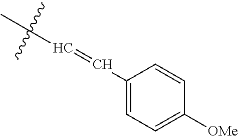 [SMILES: *C=Cc1ccc(OC)cc1] |
| 289 | S | H | 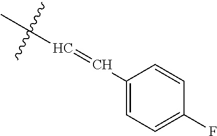 [SMILES: *C=Cc1ccc(F)cc1] |
| 290 | S | H | 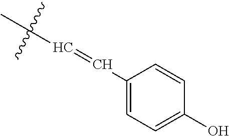 [SMILES: *C=Cc1ccc(O)cc1] |
| 291 | S | H | 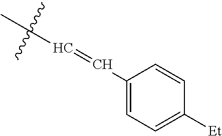 [SMILES: *C=Cc1ccc(CC)cc1] |
| 292 | S | F | 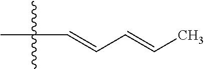 [SMILES: */C=C/C=C/C] |
| 293 | S | F | 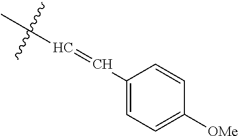 [SMILES: *C=Cc1ccc(OC)cc1] |
| 294 | S | F | 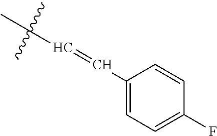 [SMILES: *C=Cc1ccc(F)cc1] |
| 295 | S | F | 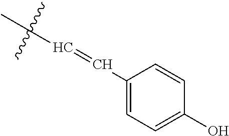 [SMILES: *C=Cc1ccc(O)cc1] |
| 296 | S | F | 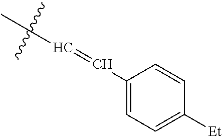 [SMILES: *C=Cc1ccc(CC)cc1] |
| 297 | S | CH₃ | 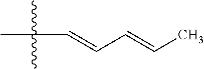 [SMILES: */C=C/C=C/C] |
| 298 | S | CH₃ | 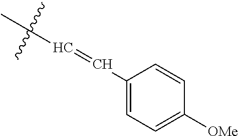 [SMILES: *C=Cc1ccc(OC)cc1] |
| 299 | S | CH₃ | 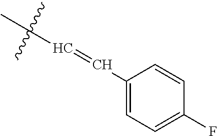 [SMILES: *C=Cc1ccc(F)cc1] |
| 300 | S | CH₃ | 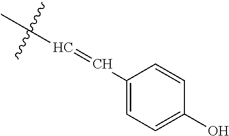 [SMILES: *C=Cc1ccc(O)cc1] |
| 301 | S | CH₃ | 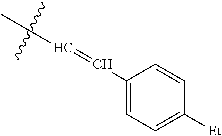 [SMILES: *C=Cc1ccc(CC)cc1] |

and pharmaceutically acceptable salts thereof.

In one embodiment, R^{7a} of Compound 258-261, 263-266, 268-271, 273-276, 278-281, 283-286, 288-291, 293-296, or 298-301 is cis. In another embodiment, R^{7a} of Compound 258-261, 263-266, 268-271, 273-276, 278-281, 283-286, 288-291, 293-296, or 298-301 is trans.

In another embodiment, the invention provides compounds of the following Formula XIV

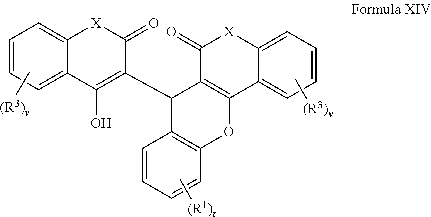

and pharmaceutically acceptable salts thereof, wherein X, R¹, R³, t, and v are as provided above in the summary of the invention for the compounds or pharmaceutically acceptable salts of Formula XIV.

In some embodiments, X is O. In some embodiments, R¹ is halo or amino. In some embodiments, R³ is fluoro or ethyl. In other embodiments, X is O and R¹ is halo or amino. In other embodiments, X is O and R³ is fluoro or ethyl. In other embodiments, X is O, R³ is fluoro or ethyl and R¹ is halo or amino.

In certain embodiments, R¹ is methoxy, ethoxy, propoxy, butoxy, pentoxy, hexoxy, heptoxy or octoxy.

In other embodiments, the compounds of Formula XIV have the Formula XIVa. In some embodiments, the compounds of Formula XIVa are those where R^{1a} and R^{1b} are independently H, halo, or amino. In some embodiments, the compounds of Formula XIVa are those where R^{3a} is fluoro or ethyl. In other embodiments, the compounds of Formula XIVa are those where R^{1a} and R^{1b} are independently H, halo, or amino and R^{3a} is fluoro or ethyl.

Illustrative examples of the compounds of Formula XIVa include those set forth below in Table 14.

TABLE 14
Illustrative examples of the compounds of Formula XIVa

| | |
|---|---|
| | Formula XIVa |
| 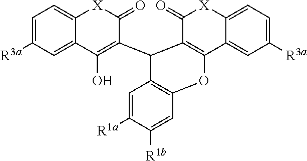 | |

| Cpd. | X | R^{1a} | R^{1b} | R^{3a} |
|---|---|---|---|---|
| 302 | O | H | H | Et |
| 303 | O | H | F | Et |
| 304 | O | H | Cl | Et |
| 305 | O | H | Br | Et |
| 306 | O | H | I | Et |
| 307 | O | H | NH₂ | Et |
| 308 | O | F | F | Et |
| 309 | O | Cl | Cl | Et |
| 310 | O | Br | Br | Et |
| 311 | O | I | I | Et |
| 312 | O | NH₂ | NH₂ | Et |
| 313 | O | H | H | F |
| 314 | O | H | F | F |
| 315 | O | H | Cl | F |
| 316 | O | H | Br | F |
| 317 | O | H | I | F |
| 318 | O | H | NH₂ | F |
| 319 | O | F | F | F |
| 320 | O | Cl | Cl | F |
| 321 | O | Br | Br | F |
| 322 | O | I | I | F |
| 323 | O | NH₂ | NH₂ | F |
| 324 | NH | H | H | Et |
| 325 | NH | H | F | Et |
| 326 | NH | H | Cl | Et |
| 327 | NH | H | Br | Et |
| 328 | NH | H | I | Et |
| 329 | NH | H | NH₂ | Et |
| 330 | NH | F | F | Et |
| 331 | NH | Cl | Cl | Et |
| 332 | NH | Br | Br | Et |
| 333 | NH | I | I | Et |
| 334 | NH | NH₂ | NH₂ | Et |
| 335 | NH | H | H | F |
| 336 | NH | H | F | F |
| 337 | NH | H | Cl | F |
| 338 | NH | H | Br | F |
| 339 | NH | H | I | F |
| 340 | NH | H | NH₂ | F |
| 341 | NH | F | F | F |
| 342 | NH | Cl | Cl | F |
| 343 | NH | Br | Br | F |
| 344 | NH | I | I | F |
| 345 | NH | NH₂ | NH₂ | F |
| 346 | S | H | H | Et |
| 347 | S | H | F | Et |
| 348 | S | H | Cl | Et |
| 349 | S | H | Br | Et |
| 350 | S | H | I | Et |
| 351 | S | H | NH₂ | Et |
| 352 | S | F | F | Et |
| 353 | S | Cl | Cl | Et |
| 354 | S | Br | Br | Et |
| 355 | S | I | I | Et |
| 356 | S | NH₂ | NH₂ | Et |
| 357 | S | H | H | F |
| 358 | S | H | F | F |
| 359 | S | H | Cl | F |
| 360 | S | H | Br | F |
| 361 | S | H | I | F |
| 362 | S | H | NH₂ | F |
| 363 | S | F | F | F |
| 364 | S | Cl | Cl | F |
| 365 | S | Br | Br | F |
| 366 | S | I | I | F |
| 367 | S | NH₂ | NH₂ | F |

and pharmaceutically acceptable salts thereof.

In another embodiment, the invention provides compounds of the following Formula XV

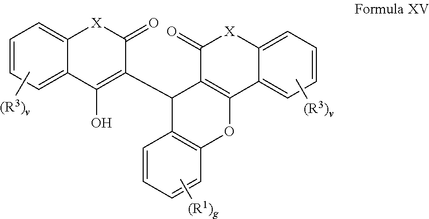

and pharmaceutically acceptable salts thereof, wherein X, R¹, R³, g, and v are as provided above in the summary of the invention for the compounds or pharmaceutically acceptable salts of Formula XV.

In some embodiments, X is O. In some embodiments R¹ is fluoro, amino, or ethoxy. In some embodiments, R³ is fluoro or methyl. In other embodiments, X is O and R¹ is fluoro, amino or ethoxy. In some embodiments, X is O and R¹ is fluoro, amino or ethoxy, and v is 0.

In other embodiments, the compounds of Formula XV have the Formula XVa, set forth below. In some embodiments, the compounds of Formula XVa are those where R^{1a} and R^{1b} are independently fluoro, amino, ethoxy, propoxy, butoxy, pentoxy, hexoxy, heptoxy or octoxy. In some embodiments, the compounds of Formula XVa are those where R^{1a} is not methoxy. In some embodiments, the compounds of Formula XVa are those where R^{1b} is not methoxy. In some embodiments, the compounds of Formula XVa are those where R^{1a} and R^{1b} are fluoro. In some embodiments, the compounds of Formula XVa are those where R^{3a} is H, fluoro, or methyl. In other embodiments, the compounds of Formula XVa are those where R^{1a} and R^{1b} are independently fluoro, amino, ethoxy, propoxy, butoxy, pentoxy, hexoxy, heptoxy or octoxy and R^{3a} is H, fluoro, or methyl.

Illustrative examples of the compounds of Formula XVa include those set forth below in Table 15.

TABLE 15
Illustrative examples of the compounds of Formula XVa

| | | | | Formula XVa |
|---|---|---|---|---|
| 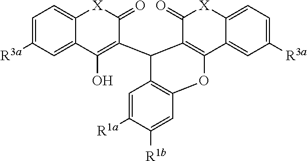 | | | | |
| Cpd. | X | R^{1a} | R^{1b} | R^{3a} |
| 368 | O | H | F | H |
| 369 | O | F | F | H |
| 370 | O | H | OEt | H |
| 371 | O | OEt | OEt | H |
| 372 | O | H | NH₂ | H |
| 373 | O | NH₂ | NH₂ | H |
| 374 | O | H | F | F |
| 375 | O | F | F | F |
| 376 | O | H | OEt | F |
| 377 | O | OEt | OEt | F |
| 378 | O | H | NH₂ | F |
| 379 | O | NH₂ | NH₂ | F |
| 380 | O | H | F | CH₃ |
| 381 | O | F | F | CH₃ |
| 382 | O | H | OEt | CH₃ |
| 383 | O | OEt | OEt | CH₃ |
| 384 | O | H | NH₂ | CH₃ |
| 385 | O | NH₂ | NH₂ | CH₃ |
| 386 | NH | H | F | H |
| 387 | NH | F | F | H |
| 388 | NH | H | OEt | H |
| 389 | NH | OEt | OEt | H |
| 390 | NH | H | NH₂ | H |
| 391 | NH | NH₂ | NH₂ | H |
| 392 | NH | H | F | F |
| 393 | NH | F | F | F |
| 394 | NH | H | OEt | F |
| 395 | NH | OEt | OEt | F |
| 396 | NH | H | NH₂ | F |
| 397 | NH | NH₂ | NH₂ | F |
| 398 | NH | H | F | CH₃ |
| 399 | NH | F | F | CH₃ |
| 400 | NH | H | OEt | CH₃ |
| 401 | NH | OEt | OEt | CH₃ |
| 402 | NH | H | NH₂ | CH₃ |
| 403 | NH | NH₂ | NH₂ | CH₃ |
| 404 | S | H | F | H |
| 405 | S | F | F | H |
| 406 | S | H | OEt | H |
| 407 | S | OEt | OEt | H |
| 408 | S | H | NH₂ | H |
| 409 | S | NH₂ | NH₂ | H |
| 410 | S | H | F | F |
| 411 | S | F | F | F |
| 412 | S | H | OEt | F |
| 413 | S | OEt | OEt | F |
| 414 | S | H | NH₂ | F |
| 415 | S | NH₂ | NH₂ | F |
| 416 | S | H | F | CH₃ |
| 417 | S | F | F | CH₃ |
| 418 | S | H | OEt | CH₃ |
| 419 | S | OEt | OEt | CH₃ |
| 420 | S | H | NH₂ | CH₃ |
| 421 | S | NH₂ | NH₂ | CH₃ |

and pharmaceutically acceptable salts thereof.

In another embodiment, the invention provides compounds of the following Formula XVI

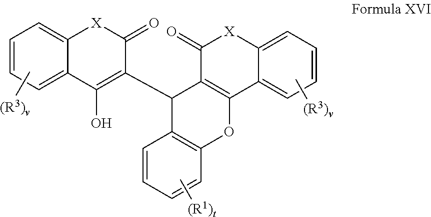

and pharmaceutically acceptable salts thereof, wherein X, R¹, R³, t, and g are as provided above in the summary of the invention for the compounds or pharmaceutically acceptable salts of Formula XVI.

In some embodiments, X is O. In some embodiments, R¹ is halo. In some embodiments, R³ is fluoro or methyl. In other embodiments, R¹ is halo and R³ is fluoro or methyl. In other embodiments, X is O, R¹ is halo, and v is 0.

In other embodiments, the compounds of Formula XVI have the Formula XVIa, set forth below. In some embodiments, the compounds of Formula XVIa are those where R^{1a} is H and R^{1b}, R^{1c}, and R^{1d} are independently halo. In some embodiments, the compounds of Formula XVIa are those where R^{1a}, R^{1b}, R^{1c}, and R^{1d} are independently halo. In some embodiments, the compounds of Formula XVIa are those where R^{1a}, R^{1b}, R^{1c}, and R^{1d} are fluoro.

Illustrative examples of the compounds of Formula XVIa include those set forth below in Table 16.

TABLE 16
Illustrative examples of the compounds of Formula XVIa

| | | | | | | Formula XVIa |
|---|---|---|---|---|---|---|
| 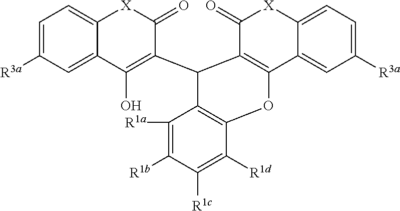 | | | | | | |
| Cpd. | X | R^{1a} | R^{1b} | R^{1c} | R^{1d} | R^{3a} |
| 422 | O | H | F | F | F | H |
| 423 | O | F | F | F | F | H |
| 424 | O | H | Cl | Cl | Cl | H |
| 425 | O | Cl | Cl | Cl | Cl | H |
| 426 | O | H | Br | Br | Br | H |
| 427 | O | Br | Br | Br | Br | H |
| 428 | O | H | F | F | F | F |
| 429 | O | F | F | F | F | F |
| 430 | O | H | Cl | Cl | Cl | F |
| 431 | O | Cl | Cl | Cl | Cl | F |
| 432 | O | H | Br | Br | Br | F |
| 433 | O | Br | Br | Br | Br | F |
| 434 | O | H | F | F | F | CH₃ |
| 435 | O | F | F | F | F | CH₃ |
| 436 | O | H | Cl | Cl | Cl | CH₃ |
| 437 | O | Cl | Cl | Cl | Cl | CH₃ |
| 438 | O | H | Br | Br | Br | CH₃ |
| 439 | O | Br | Br | Br | Br | CH₃ |
| 440 | NH | H | F | F | F | H |
| 441 | NH | F | F | F | F | H |
| 442 | NH | H | Cl | Cl | Cl | H |
| 443 | NH | Cl | Cl | Cl | Cl | H |
| 444 | NH | H | Br | Br | Br | H |
| 445 | NH | Br | Br | Br | Br | H |
| 446 | NH | H | F | F | F | F |
| 447 | NH | F | F | F | F | F |
| 448 | NH | H | Cl | Cl | Cl | F |
| 449 | NH | Cl | Cl | Cl | Cl | F |
| 450 | NH | H | Br | Br | Br | F |
| 451 | NH | Br | Br | Br | Br | F |
| 452 | NH | H | F | F | F | CH₃ |
| 453 | NH | F | F | F | F | CH₃ |
| 454 | NH | H | Cl | Cl | Cl | CH₃ |
| 455 | NH | Cl | Cl | Cl | Cl | CH₃ |
| 456 | NH | H | Br | Br | Br | CH₃ |
| 457 | NH | Br | Br | Br | Br | CH₃ |
| 458 | S | H | F | F | F | H |
| 459 | S | F | F | F | F | H |
| 460 | S | H | Cl | Cl | Cl | H |
| 461 | S | Cl | Cl | Cl | Cl | H |
| 462 | S | H | Br | Br | Br | H |
| 463 | S | Br | Br | Br | Br | H |
| 464 | S | H | F | F | F | F |
| 465 | S | F | F | F | F | F |
| 466 | S | H | Cl | Cl | Cl | F |
| 467 | S | Cl | Cl | Cl | Cl | F |
| 468 | S | H | Br | Br | Br | F |
| 469 | S | Br | Br | Br | Br | F |
| 470 | S | H | F | F | F | CH₃ |
| 471 | S | F | F | F | F | CH₃ |
| 472 | S | H | Cl | Cl | Cl | CH₃ |
| 473 | S | Cl | Cl | Cl | Cl | CH₃ |
| 474 | S | H | Br | Br | Br | CH₃ |
| 475 | S | Br | Br | Br | Br | CH₃ |

and pharmaceutically acceptable salts thereof.

In another embodiment, the invention provides compounds of the following Formula XVII

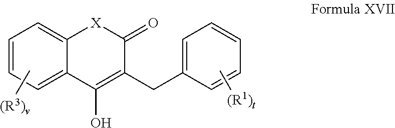

and pharmaceutically acceptable salts thereof, wherein X, R¹, R³, t, and v are as provided above in the summary of the invention for the compounds or pharmaceutically acceptable salts of Formula XVII.

In some embodiments, X is O. In some embodiments R¹ is halo. In some embodiments, R³ is fluoro or methyl. In other embodiments, X is O and R¹ is halo. In other embodiments, X is O, R¹ is halo, and R³ is fluoro or methyl.

In other embodiments, the compounds of Formula XVII have the Formula XVIIa, set forth below. In some embodiments, the compounds of Formula XVIIa are those where R^{1a}, R^{1b}, and R^{1c} are independently halo. In some embodiments, the compounds of Formula XVIIa are those where R^{1a}, R^{1b}, and R^{1c} are fluoro. In some embodiments, the compounds of Formula XVIIa are those where R^{3a} is H, fluoro, or methyl.

Illustrative examples of the compounds of Formula XVIIa include those set forth below in Table 17.

TABLE 17
Illustrative examples of the compounds of Formula XVIIa

| | | | | | Formula XVIIa |
|---|---|---|---|---|---|
| 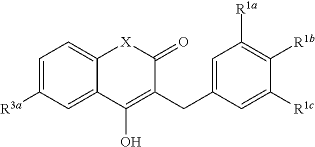 | | | | | |
| Cpd. | X | R^{1a} | R^{1b} | R^{1c} | R^{3a} |
| 476 | O | F | F | F | H |
| 477 | O | Cl | Cl | Cl | H |
| 478 | O | Br | Br | Br | H |
| 479 | O | I | I | I | H |
| 480 | O | F | F | F | CH₃ |
| 481 | O | Cl | Cl | Cl | CH₃ |
| 482 | O | Br | Br | Br | CH₃ |
| 483 | O | I | I | I | CH₃ |
| 484 | O | F | F | F | F |
| 485 | O | Cl | Cl | Cl | F |
| 486 | O | Br | Br | Br | F |
| 487 | O | I | I | I | F |
| 488 | NH | F | F | F | H |
| 489 | NH | Cl | Cl | Cl | H |
| 490 | NH | Br | Br | Br | H |
| 491 | NH | I | I | I | H |
| 492 | NH | F | F | F | CH₃ |
| 493 | NH | Cl | Cl | Cl | CH₃ |
| 494 | NH | Br | Br | Br | CH₃ |
| 495 | NH | I | I | I | CH₃ |
| 496 | NH | F | F | F | F |
| 497 | NH | Cl | Cl | Cl | F |
| 498 | NH | Br | Br | Br | F |
| 499 | NH | I | I | I | F |
| 500 | S | F | F | F | H |
| 501 | S | Cl | Cl | Cl | H |
| 502 | S | Br | Br | Br | H |
| 503 | S | I | I | I | H |
| 504 | S | F | F | F | CH₃ |
| 505 | S | Cl | Cl | Cl | CH₃ |
| 506 | S | Br | Br | Br | CH₃ |
| 507 | S | I | I | I | CH₃ |
| 508 | S | F | F | F | F |
| 509 | S | Cl | Cl | Cl | F |
| 510 | S | Br | Br | Br | F |
| 511 | S | I | I | I | F |

and pharmaceutically acceptable salts thereof.

In another embodiment, the invention provides compounds of the following Formula XVIII

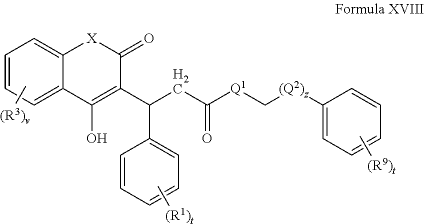

and pharmaceutically acceptable salts thereof, wherein X, R¹, R³, R⁹, Q¹, Q², t, v, and z are as provided above in the summary of the invention for the compounds or pharmaceutically acceptable salts of Formula XVIII.

In some embodiments, X is O. In some embodiments, Q¹ is NH. In some embodiments, R¹ is halo. In some embodiments, R³ is methyl. In some embodiments, R⁹ is halo. In other embodiments, X is O and Q¹ is NH. In other embodiments, X is O, Q¹ is NH, and R¹ is halo. In other embodiments, X is O, Q¹ is NH, R¹ is halo, R³ is methyl, and R⁹ is halo. In other embodiments, X is O, Q¹ is NH, R¹ is halo, R⁹ is halo, and v is 0.

In other embodiments, the compounds of Formula XVIII have the Formula XVIIIa, set forth below. In some embodiments, the compounds of Formula XVIIIa are those where R^{1a}, R^{1b}, R^{1c}, R^{9a}, and R^{9b} are independently halo. In some embodiments, the compounds of Formula XVIIIa are those where R^{1a}, R^{1b}, R^{1c}, R^{9a}, and R^{9b} are independently fluoro. In some embodiments, the compounds of Formula XVIIIa are those where R^{3a} is H or methyl.

Illustrative examples of the compounds of Formula XVIIIa include those set forth below in Table 18.

TABLE 18
Illustrative examples of the compounds of Formula XVIIIa

| | |
|---|---|
| | Formula XVIIIa |
| 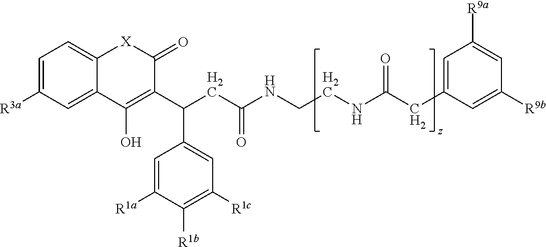 | |

| Cpd. | X | z | R^{1a} | R^{1b} | R^{1c} | R^{3a} | R^{9a} | R^{9b} |
|---|---|---|---|---|---|---|---|---|
| 512 | O | 0 | F | F | F | H | F | F |
| 513 | O | 0 | Cl | Cl | Cl | H | Cl | Cl |
| 514 | O | 0 | Br | Br | Br | H | Br | Br |
| 515 | O | 0 | I | I | I | H | I | I |
| 516 | O | 0 | F | F | F | CH₃ | F | F |
| 517 | O | 0 | Cl | Cl | Cl | CH₃ | Cl | Cl |
| 518 | O | 0 | Br | Br | Br | CH₃ | Br | Br |
| 519 | O | 0 | I | I | I | CH₃ | I | I |
| 520 | O | 1 | F | F | F | H | F | F |
| 521 | O | 1 | Cl | Cl | Cl | H | Cl | Cl |
| 522 | O | 1 | Br | Br | Br | H | Br | Br |
| 523 | O | 1 | I | I | I | H | I | I |
| 524 | O | 1 | F | F | F | CH₃ | F | F |
| 525 | O | 1 | Cl | Cl | Cl | CH₃ | Cl | Cl |
| 526 | O | 1 | Br | Br | Br | CH₃ | Br | Br |
| 527 | O | 1 | I | I | I | CH₃ | I | I |
| 528 | NH | 0 | F | F | F | H | F | F |
| 529 | NH | 0 | Cl | Cl | Cl | H | Cl | Cl |
| 530 | NH | 0 | Br | Br | Br | H | Br | Br |
| 531 | NH | 0 | I | I | I | H | I | I |
| 532 | NH | 0 | F | F | F | CH₃ | F | F |
| 533 | NH | 0 | Cl | Cl | Cl | CH₃ | Cl | Cl |
| 534 | NH | 0 | Br | Br | Br | CH₃ | Br | Br |
| 535 | NH | 0 | I | I | I | CH₃ | I | I |
| 536 | NH | 1 | F | F | F | H | F | F |
| 537 | NH | 1 | Cl | Cl | Cl | H | Cl | Cl |
| 538 | NH | 1 | Br | Br | Br | H | Br | Br |
| 539 | NH | 1 | I | I | I | H | I | I |
| 540 | NH | 1 | F | F | F | CH₃ | F | F |
| 541 | NH | 1 | Cl | Cl | Cl | CH₃ | Cl | Cl |
| 542 | NH | 1 | Br | Br | Br | CH₃ | Br | Br |
| 543 | NH | 1 | I | I | I | CH₃ | I | I |
| 544 | S | 0 | F | F | F | H | F | F |
| 545 | S | 0 | Cl | Cl | Cl | H | Cl | Cl |
| 546 | S | 0 | Br | Br | Br | H | Br | Br |
| 547 | S | 0 | I | I | I | H | I | I |
| 548 | S | 0 | F | F | F | CH₃ | F | F |
| 549 | S | 0 | Cl | Cl | Cl | CH₃ | Cl | Cl |
| 550 | S | 0 | Br | Br | Br | CH₃ | Br | Br |
| 551 | S | 0 | I | I | I | CH₃ | I | I |
| 552 | S | 1 | F | F | F | H | F | F |
| 553 | S | 1 | Cl | Cl | Cl | H | Cl | Cl |
| 554 | S | 1 | Br | Br | Br | H | Br | Br |
| 555 | S | 1 | I | I | I | H | I | I |
| 556 | S | 1 | F | F | F | CH₃ | F | F |
| 557 | S | 1 | Cl | Cl | Cl | CH₃ | Cl | Cl |
| 558 | S | 1 | Br | Br | Br | CH₃ | Br | Br |
| 559 | S | 1 | I | I | I | CH₃ | I | I |

and pharmaceutically acceptable salts therof.

In another embodiment, the invention provides compounds of the following Formula XIX

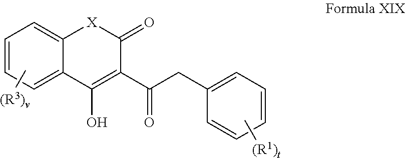

and pharmaceutically acceptable salts thereof, wherein X, R¹, R³, t, and v are as provided above in the summary of the invention for the compounds or pharmaceutically acceptable salts of Formula XIX.

In some embodiments, X is O. In some embodiments, R¹ is halo. In some embodiments, R³ is methyl. In other embodiments, X is O and R¹ is halo. In other embodiments, X is O and R³ is methyl. In other embodiments, X is O, R¹ is halo, and R³ is methyl. In some embodiments, X is O, R¹ is halo, and v is 0.

In other embodiments, the compounds of Formula XIX have the Formula XIXa, set forth below. In some embodiments, the compounds of Formula XIXa are those where R^{1a} and R^{1b} are independently halo. In some embodiments, the compounds of Formula XIXa are those where R^{1a} and R^{1b} are fluoro. In some embodiments, the compounds of Formula XIXa are those where R^{3a} is H or methyl. In other embodiments, the compounds of Formula XIXa are those where R^{1a} and R^{1b} are fluoro and R^{3a} is H or methyl.

Illustrative examples of the compounds of Formula XIXa include those set forth below in Table 19.

TABLE 19
Illustrative examples of the compounds of Formula XIXa

| | |
|---|---|
| | Formula XIXa |
| 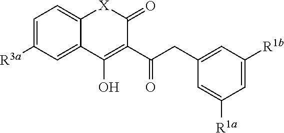 | |

| Cpd. | X | R^{1a} | R^{1b} | R^{3a} |
|---|---|---|---|---|
| 560 | O | F | F | H |
| 561 | O | Cl | Cl | H |
| 562 | O | Br | Br | H |
| 563 | O | I | I | H |
| 564 | O | F | F | CH₃ |
| 565 | O | Cl | Cl | CH₃ |
| 566 | O | Br | Br | CH₃ |
| 567 | O | I | I | CH₃ |
| 568 | NH | F | F | H |
| 569 | NH | Cl | Cl | H |
| 570 | NH | Br | Br | H |
| 571 | NH | I | I | H |
| 572 | NH | F | F | CH₃ |
| 573 | NH | Cl | Cl | CH₃ |
| 574 | NH | Br | Br | CH₃ |
| 575 | NH | I | I | CH₃ |
| 576 | S | F | F | H |
| 577 | S | Cl | Cl | H |
| 578 | S | Br | Br | H |
| 579 | S | I | I | H |
| 580 | S | F | F | CH₃ |
| 581 | S | Cl | Cl | CH₃ |
| 582 | S | Br | Br | CH₃ |
| 583 | S | I | I | CH₃ |

and pharmaceutically acceptable salts thereof.

In another embodiment, the invention provides compounds of the following Formula XX

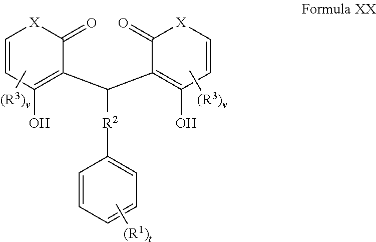

and pharmaceutically acceptable salts thereof, wherein X, R¹, R², R³, t, and v are as provided above in the summary of the invention for the compounds or pharmaceutically acceptable salts of Formula XX.

In some embodiments, X is O. In some embodiments, R¹ is halo. In some embodiments, R³ is methyl. In some embodiments, R² is —CH═CH—. In other embodiments, X is O and R¹ is halo. In other embodiments, X is O, R¹ is halo, and R³ is methyl. In other embodiments, X is O, R¹ is halo, R³ is methyl, and R² is —CH═CH—.

In other embodiments, the compounds of Formula XX have the Formula XXa, set forth below. In some embodiments, the compounds of Formula XXa are those where R^{1a} is H and R^{1b} and R^{1c} are independently halo. In other embodiments, the compounds of Formula XXa are those where R^{1a}, R^{1b}, and R^{1c} are independently halo. In other embodiments, the compounds of Formula XXa are those where R² is —CH═CH—. In some embodiments, R² is trans —CH═CH—. In other embodiments, R² is cis —CH═CH—. In other embodiments, the compounds of Formula XXa are those where R^{1a} is H, R^{1b} and R^{1c} are independently halo, and R² is —CH═CH—.

Illustrative examples of the compounds of Formula XXa include those set forth below in Table 20.

TABLE 20
Illustrative examples of the compounds of Formula XXa

| | |
|---|---|
| | Formula XXa |
| 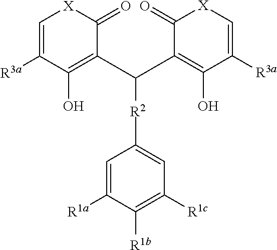 | |

| Cpd. | X | R^{1a} | R^{1b} | R^{1c} | R² | R^{3a} |
|---|---|---|---|---|---|---|
| 584 | O | H | F | F | HC═CH | H |
| 585 | O | H | Cl | Cl | HC═CH | H |
| 586 | O | H | Br | Br | HC═CH | H |
| 587 | O | H | I | I | HC═CH | H |
| 588 | O | F | F | F | HC═CH | H |
| 589 | O | Cl | Cl | Cl | HC═CH | H |
| 590 | O | Br | Br | Br | HC═CH | H |
| 591 | O | I | I | I | HC═CH | H |
| 592 | O | H | F | F | HC═CH | CH₃ |
| 593 | O | H | Cl | Cl | HC═CH | CH₃ |
| 594 | O | H | Br | Br | HC═CH | CH₃ |
| 595 | O | H | I | I | HC═CH | CH₃ |
| 596 | O | F | F | F | HC═CH | CH₃ |
| 597 | O | Cl | Cl | Cl | HC═CH | CH₃ |
| 598 | O | Br | Br | Br | HC═CH | CH₃ |
| 599 | O | I | I | I | HC═CH | CH₃ |
| 600 | S | H | F | F | HC═CH | H |
| 601 | S | H | Cl | Cl | HC═CH | H |
| 602 | S | H | Br | Br | HC═CH | H |
| 603 | S | H | I | I | HC═CH | H |
| 604 | S | F | F | F | HC═CH | H |
| 605 | S | Cl | Cl | Cl | HC═CH | H |
| 606 | S | Br | Br | Br | HC═CH | H |
| 607 | S | I | I | I | HC═CH | H |
| 608 | S | H | F | F | HC═CH | CH₃ |
| 609 | S | H | Cl | C | HC═CH | CH₃ |
| 610 | S | H | Br | Br | HC═CH | CH₃ |
| 611 | S | H | I | I | HC═CH | CH₃ |
| 612 | S | F | F | F | HC═CH | CH₃ |
| 613 | S | Cl | Cl | Cl | HC═CH | CH₃ |
| 614 | S | Br | Br | Br | HC═CH | CH₃ |
| 615 | S | I | I | I | HC═CH | CH₃ |

and pharmaceutically acceptable salts thereof.

In one embodiment, R² of compound 584-614 or 615 is cis. In another embodiment, R² of compound 584-614 or 615 is trans.

In another embodiment, the invention provides compounds of the following Formula XXI

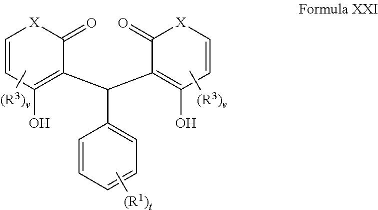

and pharmaceutically acceptable salts thereof, wherein X, R¹, R³, t, and v are as provided above in the summary of the invention for the compounds or pharmaceutically acceptable salts of Formula XXI.

In some embodiments, X is O. In some embodiments, R¹ is halo. In some embodiments, R³ is ethyl. In other embodiments, X is O and R¹ is halo. In other embodiments, X is O and R³ is ethyl. In other embodiments, X is O, R¹ is halo, and R³ is ethyl.

In other embodiments, the compounds of Formula XXI have the Formula XXIa, set forth below. In some embodiments, the compounds of Formula XXIa are those where R^{1a} is H and R^{1b} and R^{1c} are independently halo. In some embodiments, the compounds of Formula XXIa are those where R^{1a}, R^{1b}, and R^{1c} are independently halo. In some embodiments, the compounds of Formula XXIa are those where R^{3a} is H or ethyl. In other embodiments, the compounds of Formula XXIa are those where R^{1a} is H, R^{1b} and R^{1c} are independently halo, and R^{3a} is H or ethyl.

Illustrative examples of the compounds of Formula XXIa include those set forth below in Table 21.

TABLE 21
Illustrative examples of the compounds of Formula XXIa

| | |
|---|---|
| | Formula XXIa |
| 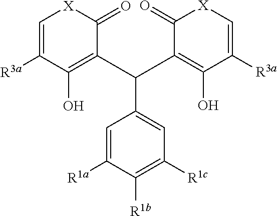 | |

| Cpd. | X | R^{1a} | R^{1b} | R^{1c} | R^{3a} |
|---|---|---|---|---|---|
| 616 | O | H | F | F | H |
| 617 | O | H | Cl | Cl | H |
| 618 | O | H | Br | Br | H |
| 619 | O | H | I | I | H |
| 620 | O | F | F | F | H |
| 621 | O | Cl | Cl | Cl | H |
| 622 | O | Br | Br | Br | H |
| 623 | O | I | I | I | H |
| 624 | O | H | F | F | Et |
| 625 | O | H | Cl | Cl | Et |
| 626 | O | H | Br | Br | Et |
| 627 | O | H | I | I | Et |
| 628 | O | F | F | F | Et |
| 629 | O | Cl | Cl | Cl | Et |
| 630 | O | Br | Br | Br | Et |
| 631 | O | I | I | I | Et |
| 632 | S | H | F | F | H |
| 633 | S | H | Cl | Cl | H |
| 634 | S | H | Br | Br | H |
| 635 | S | H | I | I | H |
| 636 | S | F | F | F | H |
| 637 | S | Cl | Cl | Cl | H |
| 638 | S | Br | Br | Br | H |
| 639 | S | I | I | I | H |
| 640 | S | H | F | F | Et |
| 641 | S | H | Cl | Cl | Et |
| 642 | S | H | Br | Br | Et |
| 643 | S | H | I | I | Et |
| 644 | S | F | F | F | Et |
| 645 | S | Cl | Cl | Cl | Et |
| 646 | S | Br | Br | Br | Et |
| 647 | S | I | I | I | Et |

and pharmaceutically acceptable salts thereof.

In another embodiment, the invention provides compounds of the following Formula XXII

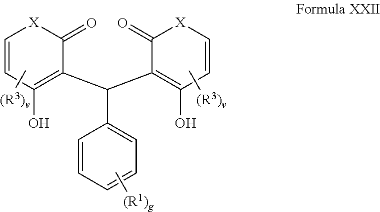

and pharmaceutically acceptable salts thereof, wherein X, R¹, R³, g, and v are as provided above in the summary of the invention for the compounds and pharmaceutically acceptable salts of Formula XXII.

In some embodiments, X is O. In some embodiments, R¹ is halo. In some embodiments, R³ is ethyl. In other embodiments, X is O and R¹ is halo. In other embodiments, X is O and R³ is ethyl. In other embodiments, X is O, R¹ is halo, and R³ is ethyl.

In other embodiments, the compounds of Formula XXII have the Formula XXIIa, set forth below. In some embodiments, the compounds of Formula XXIIa are those where R^{1a} is H and R^{1b} and R^{1c} are independently halo or methyl. In some embodiments, the compounds of Formula XXIIa are those where R^{1a}, R^{1b}, and R^{1c} are independently halo or methyl. In some embodiments, the compounds of Formula XXIIa are those where R^{3a} is H or ethyl. In other embodiments, the compounds of Formula XXIIa are those where R^{1a} is H, R^{1b} and R^{1c} are independently halo or methyl, and R^{3a} is H or ethyl.

Illustrative examples of the compounds of Formula XXIIa include those set forth below in Table 22.

TABLE 22
Illustrative examples of the compounds of Formula XXIIa

| | |
|---|---|
| | Formula XXIIa |
| 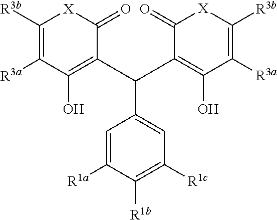 | |

| Cpd. | X | R^{1a} | R^{1b} | R^{1c} | R^{3a} | R^{3b} |
|---|---|---|---|---|---|---|
| 648 | O | H | CH₃ | CH₃ | H | H |
| 649 | O | H | CH₃ | CH₃ | Et | Et |
| 650 | O | H | F | F | Et | Et |
| 651 | O | H | Cl | Cl | Et | Et |
| 652 | O | H | Br | Br | Et | Et |
| 653 | O | H | I | I | Et | Et |
| 654 | O | F | F | F | Et | Et |
| 655 | O | Cl | Cl | Cl | Et | Et |
| 656 | O | Br | Br | Br | Et | Et |
| 657 | O | I | I | I | Et | Et |
| 658 | S | H | CH₃ | CH₃ | H | H |
| 659 | S | H | CH₃ | CH₃ | Et | Et |
| 660 | S | H | F | F | Et | Et |
| 661 | S | H | Cl | Cl | Et | Et |
| 662 | S | H | Br | Br | Et | Et |
| 663 | S | H | I | I | Et | Et |
| 664 | S | F | F | F | Et | Et |
| 665 | S | Cl | Cl | Cl | Et | Et |
| 666 | S | Br | Br | Br | Et | Et |
| 667 | S | I | I | I | Et | Et |

and pharmaceutically acceptable salts thereof.

In another embodiment, the invention provides compounds of the following Formula XXIII

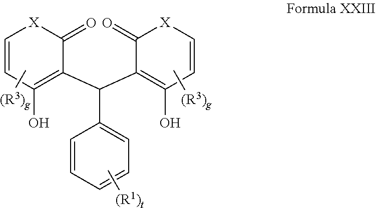

and pharmaceutically acceptable salts thereof, wherein X, R¹, R³, t, and g are as provided above in the summary of the invention for the compounds or pharmaceutically acceptable salts of Formula XXIII.

In some embodiments, X is O. In some embodiments, R¹ is halo. In some embodiments, R³ is fluoro or methyl. In other embodiments, X is O and R¹ is halo. In other embodiments, X is O and R³ is fluoro or methyl. In other embodiments, X is O, R¹ is halo, and R³ is fluoro or methyl.

In other embodiments, the compounds of Formula XXIII have the Formula XXIIIa, set forth below. In some embodiments, the compounds of Formula XXIIIa are those where R^{1a}, R^{1b}, and R^{1c} are independently halo. In some embodiments, the compounds of Formula XXIIIa are those where R^{1a}, R^{1b}, and R^{1c} are fluoro. In some embodiments, the compounds of Formula XXIIIa are those where R^{3a} is fluoro or methyl. In other embodiments, the compounds of Formula XXIIIa are those where R^{1a}, R^{1b}, and R^{1c} are independently halo and R^{3a} is fluoro or methyl.

Illustrative examples of the compounds of Formula XXIIIa include those set forth below in Table 23.

TABLE 23
Illustrative examples of the compounds of Formula XXIIIa

| | |
|---|---|
| | Formula XXIIIa |
| 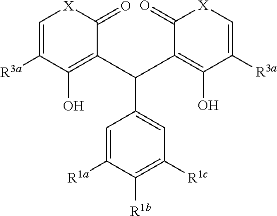 | |

| Cpd. | X | R^{1a} | R^{1b} | R^{1c} | R^{3a} |
|---|---|---|---|---|---|
| 668 | O | F | F | F | CH₃ |
| 669 | O | Cl | Cl | Cl | CH₃ |
| 670 | O | Br | Br | Br | CH₃ |
| 671 | O | I | I | I | CH₃ |
| 672 | O | F | F | F | F |
| 673 | O | Cl | Cl | Cl | F |
| 674 | O | Br | Br | Br | F |
| 675 | O | I | I | I | F |
| 676 | S | F | F | F | CH₃ |
| 677 | S | Cl | Cl | Cl | CH₃ |
| 678 | S | Br | Br | Br | CH₃ |
| 679 | S | I | I | I | CH₃ |
| 680 | S | F | F | F | F |
| 681 | S | Cl | Cl | Cl | F |
| 682 | S | Br | Br | Br | F |
| 683 | S | I | I | I | F |

and pharmaceutically acceptable salts thereof.

In another embodiment, the invention provides compounds of the following Formula XXIV

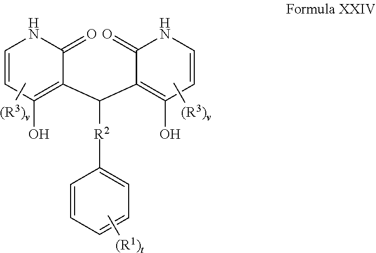

and pharmaceutically acceptable salts thereof, wherein R¹, R², R³, t, and v are as provided above in the summary of the invention for the compounds or pharmaceutically acceptable salts of Formula XXIV.

In some embodiments, R¹ is halo. In some embodiments, R² is —CH═CH—. In some embodiments, R³ is methyl. In other embodiments R¹ is halo and R² is C₂ alkylene. In other embodiments, R¹ is halo and R³ is methyl. In other embodiments, R¹ is halo, R² is C₂ alkylene and R³ is methyl.

In other embodiments, the compounds of Formula XXIV have the Formula XXIVa, set forth below. In some embodiments, the compounds of Formula XXIVa are those where R^{1a} is H and R^{1b} and R^{1c} are independently halo. In some embodiments, the compounds of Formula XXIVa are those where R^{1a}, R^{1b}, and R^{1c} are independently halo. In some embodiments, the compounds of Formula XXIVa are those where R^{3a} is methyl and R^{3b} is H. In some embodiments, the compounds of Formula XXIVa are those where R^{3a} and R^{3b} are methyl. In other embodiments, the compounds of Formula XXIVa are those where R^{1a} is H, R^{1b} and R^{1c} are independently halo, and R^{3a} and R^{3b} are methyl.

Illustrative examples of the compounds of Formula XXIVa include those set forth below in Table 24.

TABLE 24
Illustrative examples of the compounds of Formula XXIVa

| | | | | | Formula XXIVa | |
|---|---|---|---|---|---|---|
| 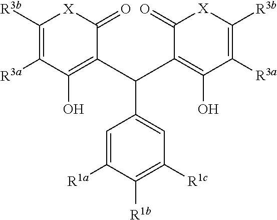 | | | | | | |
| Cpd. | R^{1a} | R^{1b} | R^{1c} | R² | R^{3a} | R^{3b} |
| 684 | H | F | F | HC═CH | CH₃ | H |
| 685 | H | Cl | Cl | HC═CH | CH₃ | H |
| 686 | H | Br | Br | HC═CH | CH₃ | H |
| 687 | H | I | I | HC═CH | CH₃ | H |
| 688 | F | F | F | HC═CH | CH₃ | H |
| 689 | Cl | Cl | Cl | HC═CH | CH₃ | H |
| 690 | Br | Br | Br | HC═CH | CH₃ | H |
| 691 | I | I | I | HC═CH | CH₃ | H |
| 692 | H | F | F | HC═CH | CH₃ | CH₃ |
| 693 | H | Cl | Cl | HC═CH | CH₃ | CH₃ |
| 694 | H | Br | Br | HC═CH | CH₃ | CH₃ |
| 695 | H | I | I | HC═CH | CH₃ | CH₃ |
| 696 | F | F | F | HC═CH | CH₃ | CH₃ |
| 697 | Cl | Cl | Cl | HC═CH | CH₃ | CH₃ |
| 698 | Br | Br | Br | HC═CH | CH₃ | CH₃ |
| 699 | I | I | I | HC═CH | CH₃ | CH₃ |

and pharmaceutically acceptable salts thereof.

In one embodiment, R² of Compound 684-698 or 699 is cis. In another embodiment, R² of Compound 684-698 or 699 is trans.

In another embodiment, the invention provides compounds of the following Formula XXV

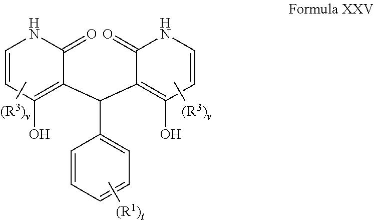

and pharmaceutically acceptable salts thereof, wherein R¹, R³, t, and v are as provided above in the summary of the invention for the compounds or pharmaceutically acceptable salts of Formula XXV.

In some embodiments, R¹ is halo. In some embodiments, R¹ is fluoro, chloro, bromo, or iodo. In some embodiments, R³ is ethyl, propyl, or butyl. In other embodiments, R¹ is halo and R³ is ethyl.

In other embodiments, the compounds of Formula XXV have the Formula XXVa, set forth below. In some embodiments, the compounds of Formula XXVa are those where R^{1a} is H and R^{1b} and R^{1c} are independently halo. In some embodiments, the compounds of Formula XXVa are those where R^{1a}, R^{1b}, and R^{1c} are independently halo. In some embodiments, the compounds of Formula XXVa are those where R^{3a} is ethyl and R^{3b} is H. In some embodiments, the compounds of Formula XXVa are those where R^{3a} and R^{3b} are ethyl. In other embodiments, the compounds of Formula XXVa are those where R^{1a} is H, R^{1b} and R^{1c} are independently halo, and R^{3a} and R^{3b} are ethyl.

Illustrative examples of the compounds of Formula XXVa include those set forth below in Table 25.

TABLE 25
Illustrative examples of the compounds of Formula XXVa

| | | | | Formula XXVa | |
|---|---|---|---|---|---|
| 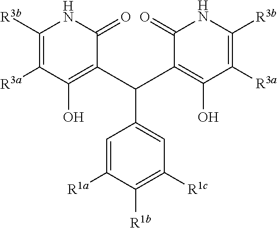 | | | | | |
| Cpd. | R^{1a} | R^{1b} | R^{1c} | R^{3a} | R^{3b} |
| 700 | H | F | F | Et | H |
| 701 | H | Cl | Cl | Et | H |
| 702 | H | Br | Br | Et | H |
| 703 | H | I | I | Et | H |
| 704 | F | F | F | Et | H |
| 705 | Cl | Cl | Cl | Et | H |
| 706 | Br | Br | Br | Et | H |
| 707 | I | I | I | Et | H |
| 708 | H | F | F | Et | Et |
| 709 | H | Cl | Cl | Et | Et |
| 710 | H | Br | Br | Et | Et |
| 711 | H | I | I | Et | Et |
| 712 | F | F | F | Et | Et |
| 713 | Cl | Cl | Cl | Et | Et |
| 714 | Br | Br | Br | Et | Et |
| 715 | I | I | I | Et | Et |

and pharmaceutically acceptable salts thereof.

In another embodiment, the invention provides compounds of the following Formula XXVI

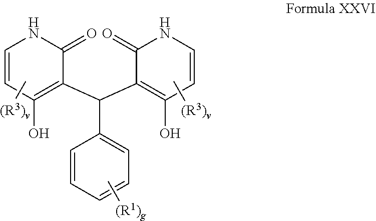

and pharmaceutically acceptable salts thereof, wherein R¹, R³, g, and v are as provided above in the summary of the invention for the compounds or pharmaceutically acceptable salts of Formula XXVI.

In some embodiments, R¹ is halo. In some embodiments, R³ is methyl. In other embodiments, R¹ is halo and R³ is methyl.

In other embodiments, the compounds of Formula XXVI have the Formula XXVIa, set forth below. In some embodiments, the compounds of Formula XXIVa are those where R^{1a} is H and R^{1b} and R^{1c} are independently fluoro, bromo, or iodo. In some embodiments, the compounds of Formula XXVIa are those where R^{1a}, R^{1b}, and R^{1c} are independently fluoro, bromo, or iodo. In some embodiments, the compounds of Formula XXVIa are those where R^{1a} is methyl and R^{3b} is H. In some embodiments, the compounds of Formula XXVIa are those where R^{3a} and R^{3b} are methyl. In some embodiments, the compounds of Formula XXIVa are those where R^{1a} is H, R^{1b} and R^{1c} are independently fluoro, bromo, or iodo, and R^{1a} and R^{3b} are methyl.

Illustrative examples of the compounds of Formula XXVIa include those set forth below in Table 26.

TABLE 26
Illustrative examples of the compounds of Formula XXVIa

| | |
|---|---|
| | Formula XXVIa |
| 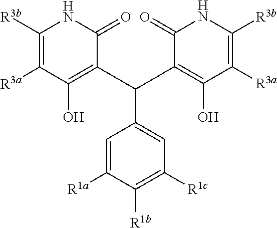 | |

| Cpd. | R^{1a} | R^{1b} | R^{1c} | R^{3a} | R^{3b} |
|---|---|---|---|---|---|
| 716 | H | F | F | CH₃ | H |
| 717 | H | Br | Br | CH₃ | H |
| 718 | H | I | I | CH₃ | H |
| 719 | F | F | F | CH₃ | H |
| 720 | Br | Br | Br | CH₃ | H |
| 721 | I | I | I | CH₃ | H |
| 722 | H | F | F | CH₃ | CH₃ |
| 723 | H | Br | Br | CH₃ | CH₃ |
| 724 | H | I | I | CH₃ | CH₃ |
| 725 | F | F | F | CH₃ | CH₃ |
| 726 | Br | Br | Br | CH₃ | CH₃ |
| 727 | I | I | I | CH₃ | CH₃ |

and pharmaceutically acceptable salts thereof.

### III. Methods for Making the Coumarin-Based Compounds

Coumarin-Based Compounds as provided herein can typically be prepared using commercially available starting reagents employing modifications to procedures known to those skilled in the art. Exemplified syntheses are set forth in the Examples below. A generalized synthesis for preparing compounds such as those of Formulas I to VI, VII, IX to XI, and XIII is provided in Scheme 1 below, in which an appropriately substituted (or nonsubstituted) 4-hydroxy coumarin or quinolin-2-one derivative is reacted with an appropriately substituted (or nonsubstituted) benzaldehyde.

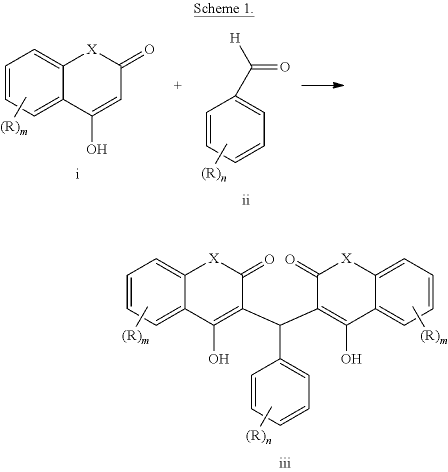

wherein X is O, NH, or S, each R is independently a substituent as described above, for instance, in Formulas I to VI, VII, IX to XI, and XIII, m is an integer from 0 to 4, and n is an integer from 0 to 5.

Typically, a solution of a compound of Formula i (2 mole equivalents) in a solvent is prepared. A compound of Formula ii (1 mole equivalent) is then added to the solution, and the resultant mixture is refluxed for a period of time sufficient to provide a compound of Formula iii. The compound of Formula iii can be isolated from the reaction mixture and purified.

The compound of Formula iii may be isolated from the reaction mixture by any method known to one of skill in the art. Such methods include, but are not limited to, filtration, chromatography or solvent extraction. The isolated compound of Formula iii may optionally be purified by any method known to one of skill in the art. Such methods include, but are not limited to, crystallization.

### IV. Treatment or Prevention of a Condition with the Coumarin-Based Compounds

In accordance with the invention, a Coumarin-Based Compound is useful for treatment or prevention of a Condition as set forth below.

A. Treatment or Prevention of Cancer

The Coumarin-Based Compounds are useful for treating or preventing cancer. Accordingly, the invention provides methods for treating or preventing cancer, comprising administering an effective amount of a Coumarin-Based Compound to a subject. In one embodiment, the subject is in need of treatment or prevention of the cancer. In one embodiment, the methods further comprise administering an effective amount of another anticancer agent. Examples of cancers that the Coumarin-Based Compounds disclosed herein are useful for treating or preventing include, but are not limited to, the cancers disclosed below in Table 27 and metastases thereof.

TABLE 27
Solid tumors, including but not limited to:

| | |
|---|---|
| fibrosarcoma | basal cell carcinoma |
| myxosarcoma | adenocarcinoma |
| liposarcoma | sweat gland carcinoma |
| chondrosarcoma | sebaceous gland carcinoma |
| osteogenic sarcoma | papillary carcinoma |
| chordoma | papillary adenocarcinomas |
| angiosarcoma | cystadenocarcinoma |
| endotheliosarcoma | medullary carcinoma |
| lymphangiosarcoma | bronchogenic carcinoma |
| lymphangioendotheliosarcoma | renal cell carcinoma |
| synovioma | hepatoma |
| mesothelioma | bile duct carcinoma |
| Ewing's tumor | choriocarcinoma |
| leiomyosarcoma | seminoma |
| rhabdomyosarcoma | embryonal carcinoma |
| colon cancer | Wilms' tumor |
| colorectal cancer | cervical cancer |
| kidney cancer | uterine cancer |
| pancreatic cancer | testicular cancer |
| bone cancer | small cell lung carcinoma |
| breast cancer | bladder carcinoma |
| ovarian cancer | lung cancer |
| prostate cancer | epithelial carcinoma |
| esophageal cancer | skin cancer |
| stomach cancer | melanoma |
| oral cancer | metastatic melanoma |
| nasal cancer | neuroblastoma |
| throat cancer | retinoblastoma |
| squamous cell carcinoma | |

Blood-borne cancers, including but not limited to:

| | |
|---|---|
| acute lymphoblastic leukemia | acute myelomonocytic leukemia |
| (“ALL”) | |
| acute lymphoblastic B-cell | acute nonlymphocyctic leukemia |
| leukemia | |
| acute lymphoblastic T-cell | acute undifferentiated leukemia |
| leukemia | |
| acute myeloblasts leukemia | chronic myelocytic leukemia |
| (“AML”) | (“CML”) |
| acute promyelocyte leukemia | chronic lymphocytic leukemia |
| (“APL”) | (“CLL”) |
| acute monoblastic leukemia | hairy cell leukemia |
| acute erythroleukemic leukemia | multiple myeloma |
| acute megakaryoblastic leukemia | |

Acute and chronic leukemias, including but not limited to:

| | |
|---|---|
| lymphoblastic | lymphocytic |
| myelogenous | myelocytic leukemias |

CNS and brain cancers, including but not limited to:

| | |
|---|---|
| glioma | acoustic neuroma |
| pilocytic astrocytoma | oligodendroglioma |
| astrocytoma | meningioma |
| anaplastic astrocytoma | vestibular schwannoma |
| glioblastoma multiforme | adenoma |
| medulloblastoma | metastatic brain tumor |
| craniopharyngioma | meningioma |
| ependymoma | spinal tumor |
| pinealoma | medulloblastoma |
| hemangioblastoma | |

In one embodiment, the cancer is lung cancer, breast cancer, colorectal cancer, prostate cancer, a leukemia, a lymphoma, non-Hodgkin's lymphoma, skin cancer, a brain cancer, a cancer of the central nervous system, ovarian cancer, uterine cancer, stomach cancer, pancreatic cancer, esophageal cancer, kidney cancer, liver cancer, or a head and neck cancer. In another embodiment, the cancer is metastatic cancer.

In yet another embodiment, the cancer is brain cancer or melanoma. In one embodiment, the brain cancer is metastatic brain cancer or a glioma. In one embodiment, the glioma is pilocytic astrocytoma, astrocytoma, anaplastic astrocytoma or glioblastoma multiforme. In one embodiment, the cancer is homologous-recombination deficient, such as BRCA-I or BRCA-2 deficient, or is deficient in one or more proteins of the Fanconi family. In one embodiment, the deficiency is caused by a genetic mutation. In another embodiment, the phenotype resulting from the deficiency is caused by abnormally low expression of BRCA-I or BRCA-2 protein. In another embodiment, the phenotype resulting from the deficiency is caused by abnormally low expression of one or more proteins of the Fanconi family.

In another embodiment, the cancer is leukemia, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemia, such as but not limited to, chronic myelocytic (granulocytic) leukemia, chronic lymphocytic leukemia, hairy cell leukemia; polycythemia vera; lymphoma such as but not limited to Hodgkin's disease, non-Hodgkin's disease; multiple myeloma such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; dendritic cell cancer, including plasmacytoid dendritic cell cancer, NK blastic lymphoma (also known as cutaneous NK/T-cell lymphoma and agranular (CD4+/CD56+) dermatologic neoplasms); basophilic leukemia; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angio sarcoma (hemangiosarcoma), fibrosarcoma, Kaposi's sarcoma, leiomyosarcoma, liposarcoma, lymphangio sarcoma, neurilemmoma, rhabdomyosarcoma, synovial sarcoma; a brain tumor such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancer such as but limited to Cushing's disease, prolactin-secreting tumor, acromegaly, and diabetes insipius; eye cancer such as but not limited to ocular melanoma such as iris melanoma, choroidal melanoma, and cilliary body melanoma, and retinoblastoma; vaginal cancer such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancer such as but not limited to, squamous cell carcinoma, and adenocarcinoma; uterine cancer such as but not limited to endometrial carcinoma and uterine sarcoma; ovarian cancer such as but not limited to, ovarian epithelial carcinoma, borderline tumor, germ cell tumor, and stromal tumor; esophageal cancer such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancer such as but not limited to, adenocarcinoma, fungating (polypoid), ulcerating, superficial spreading, diffusely spreading, malignant lymphoma, lipo sarcoma, fibro sarcoma, and carcinosarcoma; colon cancer; rectal cancer; liver cancer such as but not limited to hepatocellular carcinoma and hepatoblastoma; gallbladder cancer such as adenocarcinoma; cholangiocarcinomas such as but not limited to papillary, nodular, and diffuse; lung cancer such as non-small cell lung cancer, squamous cell carcinoma (epidermoid carcinoma), adenocarcinoma, large-cell carcinoma and small-cell lung cancer; testicular cancer such as but not limited to germinal tumor, seminoma, anaplastic, classic (typical), spermatocytic, nonseminoma, embryonal carcinoma, teratoma carcinoma, choriocarcinoma (yolk-sac tumor), prostate cancer such as but not limited to, prostatic intraepithelial neoplasia, adenocarcinoma, leiomyosarcoma, and rhabdomyosarcoma; penile cancer; oral cancer such as but not limited to squamous cell carcinoma; basal cancer; salivary gland cancer such as but not limited to adenocarcinoma, mucoepidermoid carcinoma, and adenoidcystic carcinoma; pharynx cancer such as but not limited to squamous cell cancer, and verrucous; skin cancer such as but not limited to, basal cell carcinoma, squamous cell carcinoma and melanoma, superficial spreading melanoma, nodular melanoma, lentigo malignant melanoma, acral lentiginous melanoma; kidney cancer such as but not limited to renal cell carcinoma, adenocarcinoma, hypernephroma, fibrosarcoma, transitional cell cancer (renal pelvis and/or uterer); Wilms' tumor; bladder cancer such as but not limited to transitional cell carcinoma, squamous cell cancer, adenocarcinoma, carcinosarcoma. In addition, cancer include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In a specific of this embodiment, the cancer is one that is associated with cleavage of notch by γ-secretase including, but not limited to, leukemia, non small cell lung cancer, ovarian cancer, breast cancer, or brain cancer.

In still another embodiment, the subject in need of treatment has previously undergone or is presently undergoing treatment for cancer. The treatment includes, but is not limited to, chemotherapy, radiation therapy, surgery or immunotherapy, such as administration of a cancer vaccine.

In still another embodiment, the subject in need of treatment has previously undergone or is presently undergoing treatment for cancer. The treatment includes, but is not limited to, chemotherapy, radiation therapy, surgery or immunotherapy, such as administration of a cancer vaccine.

The Coumarin-Based Compounds are also useful for treating or preventing a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J. Pathol. (2003) 199(2):140-5); hepatitis B or C virus, which can lead to liver carcinoma (see, e.g., El-Serag, J. Clin. Gastroenterol. (2002) 35(5 Suppl. 2):572-8); human T cell leukemia virus (HTLV)-I, which can lead to T-cell leukemia (see, e.g., Mortreux et al., Leukemia (2003) 17(1):26-38); human herpesvirus-8 infection, which can lead to Kaposi's sarcoma (see, e.g., Kadow et al., Curr. Opin. Investig. Drugs (2002) 3(11): 1574-9); and Human Immune deficiency Virus (HIV) infection, which can lead to cancer as a consequence of immunodeficiency (see, e.g., Dal Maso et al., Lancet Oncol (2003) 4(2): 110-9). Each of these references is incorporated herein by reference.

The Coumarin-Based Compounds are also useful for preventing cancer, or preventing progression of a cancer, including but not limited to the cancers listed in Table 27. Such prophylactic use includes that in which non-neoplastic cell growth such as hyperplasia, metaplasia, or most specifically, dysplasia has occurred. Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic or therapeutic administration of a Coumarin-Based Compound. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. In a specific embodiment, leukoplakia, a benign-appearing hyperplastic or dysplastic lesion of the epithelium, or Bowen's disease, a carcinoma in situ, is treatable or preventable according to the present methods.

In another embodiment, fibrocystic disease (cystic hyperplasia, mammary dysplasia, specifically adenosis (benign epithelial hyperplasia)) is treatable or preventable according to the present methods.

In other embodiments, a subject that has one or more of the following predisposing factors for malignancy can be treated by administration of an effective amount of a Coumarin-Based Compound: a chromosomal translocation associated with a malignancy (e.g., the Philadelphia chromosome for chronic myelogenous leukemia; t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine. adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome); and exposure to carcinogens (e.g., smoking, second-hand smoke exposure, and inhalation of or contacting with certain chemicals).

1. Coumarin-Based Compounds Useful for Treatment or Prevention of Cancer

In one embodiment, the Coumarin-Based Compounds that are useful for treating or preventing cancer are those of Formulas I to XXVI, described above.

In another embodiment, the invention provides methods for treating or preventing cancer, comprising administering to a subject an effective amount of a compound of Formula A

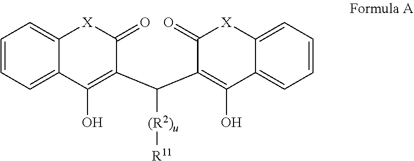

or a pharmaceutically acceptable salt thereof, wherein X, R², u, and R¹¹ are as set forth above for compounds or pharmaceutically acceptable salts of Formula A. In one embodiment, the subject is in need of treatment or prevention of cancer.

In some embodiments, the compounds of Formula A are those where u is 0 and R¹¹ is

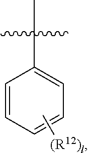

wherein each R¹² is independently bromo, iodo, C₄-C₈ alkoxy, amino, hydroxy, C₁-C₈ alkyl, NHAc, or trihalomethyl and l is 1. In certain embodiments, R¹² is independently bromo, iodo, NHAc, or trihalomethyl and l is 1.

In other embodiments, the compounds of Formula A are those where u is 0 and R¹¹ is a C₁-C₈ alkyl or C₃-C₈ cycloalkyl.

In other embodiments, the compounds of Formula A are those where u is 0 and R¹¹ is

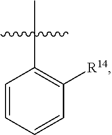

wherein R¹⁴ is bromo, iodo, or fluoro.

Illustrative examples of the compounds of Formula A include the following compounds:

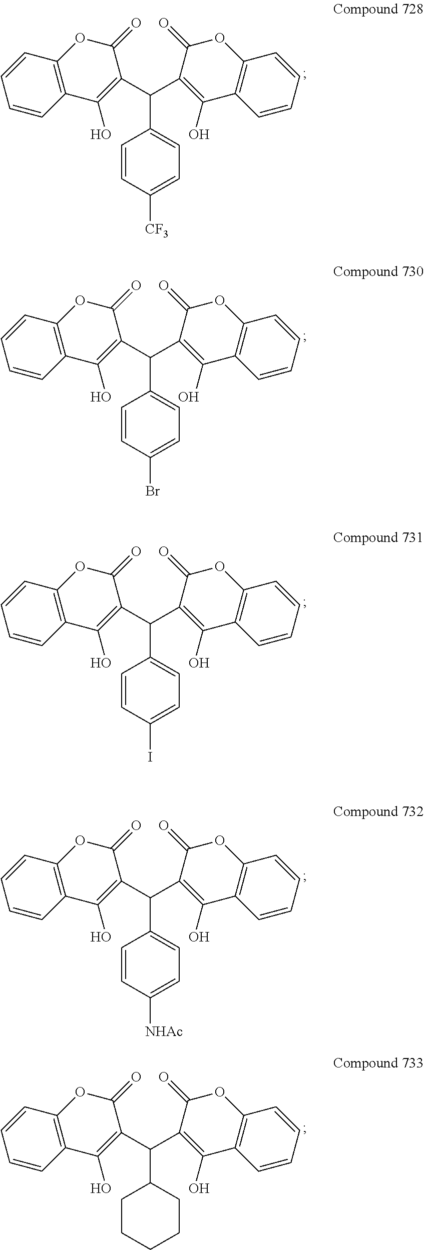 [SMILES: O=c1oc2ccccc2c(O)c1C(c1ccc(C(F)(F)F)cc1)c1c(O)c2ccccc2oc1=O ; O=c1oc2ccccc2c(O)c1C(c1ccc(Br)cc1)c1c(O)c2ccccc2oc1=O ; O=c1oc2ccccc2c(O)c1C(c1ccc(I)cc1)c1c(O)c2ccccc2oc1=O ; CC(=O)Nc1ccc(C(c2c(O)c3ccccc3oc2=O)c2c(O)c3ccccc3oc2=O)cc1 ; O=c1oc2ccccc2c(O)c1C(c1c(O)c2ccccc2oc1=O)C1CCCCC1]

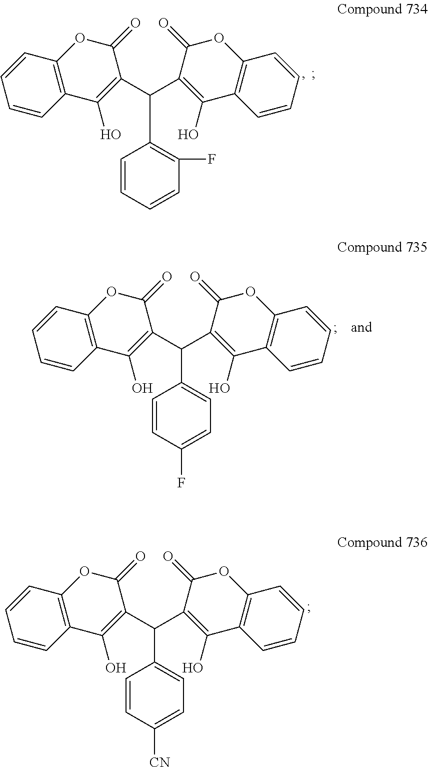 [SMILES: O=c1oc2ccccc2c(O)c1C(c1ccccc1F)c1c(O)c2ccccc2oc1=O ; O=c1oc2ccccc2c(O)c1C(c1ccc(F)cc1)c1c(O)c2ccccc2oc1=O ; N#Cc1ccc(C(c2c(O)c3ccccc3oc2=O)c2c(O)c3ccccc3oc2=O)cc1]

and pharmaceutically acceptable salts thereof.

In other embodiments, the invention provides methods for treating or preventing cancer, comprising administering to a subject an effective amount of a compound of Formula B

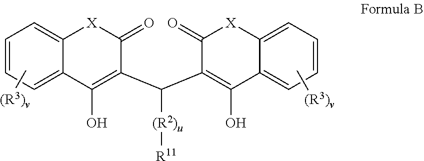

or a pharmaceutically acceptable salt thereof, wherein X, R², u, R³, v, and R¹¹ are as provided above in the summary of the invention for the compounds or pharmaceutically acceptable salts of Formula B. In one embodiment, the subject is in need of treatment or prevention of cancer.

In some embodiments, the compounds of Formula B are those where u is 0; R³ is halo or methyl; and R¹¹ is

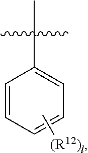

wherein each R¹² is independently bromo, fluoro, iodo, NHAc, or trihalomethyl and l is 1.

In other embodiments, the compounds of Formula B are those where u is 0; R³ is halo or methyl; and R¹¹ is a C₁-C₈ alkyl or C₃-C₈ cycloalkyl.

In certain embodiments, the compound of Formula B is

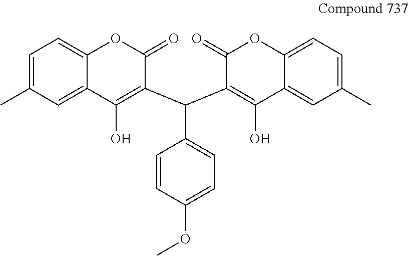

or a pharmaceutically salt thereof.

In other embodiments, the invention provides methods for treating or preventing cancer, comprising administering to a subject an effective amount of a compound of Formula C

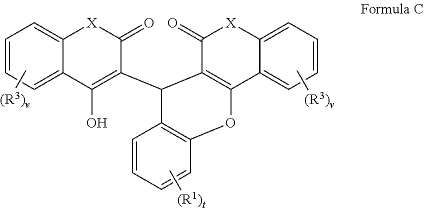

or a pharmaceutically acceptable salt thereof, wherein, X, R¹, R³, t, and v are as set forth above for the compounds or pharmaceutically acceptable salts of Formula C. In one embodiment, the subject is in need of treatment or prevention of cancer.

In some embodiments, the compounds of Formula C are those where R¹ is halo. In other embodiments, the compounds of Formula C are those where R¹ is fluoro. In other embodiments, the compounds of Formula C are those where R³ is halo or methyl. In other embodiments, the compounds of Formula C are those where R¹ is halo and R³ is halo or methyl.

In other embodiments, the invention provides methods for treating or preventing cancer, comprising administering to a subject an effective amount of a compound of Formula D

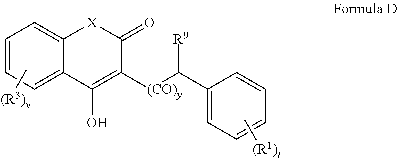

or a pharmaceutically acceptable salt thereof, wherein: X, R¹, R³, R⁹, R¹⁰, Q¹, Q², t, v, y, and z are as provided above in the summary of the invention for the compounds or pharmaceutically acceptable salts of Formula D. In one embodiment, the subject is in need of treatment or prevention of cancer.

In some embodiments, the compounds of Formula D are those where R¹ is halo. In other embodiments, the compounds of Formula D are those where R¹ is fluoro. In other embodiments, the compounds of Formula D are those where R³ is halo or methyl. In other embodiments, the compounds of Formula D are those where R¹ is halo and R³ is halo or methyl.

In other embodiments, the invention provides methods for treating or preventing cancer, comprising administering to a subject an effective amount of a compound of Formula E

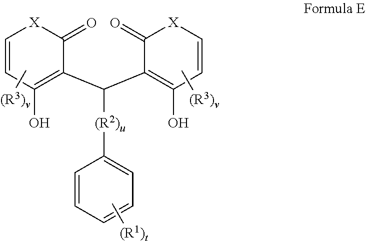

or a pharmaceutically acceptable salt thereof, wherein X, R¹, R², R³, t, v, and u are as provided above in the summary of the invention for the compounds or pharmaceutically acceptable salts of Formula E. In one embodiment, the subject is in need of treatment or prevention of cancer.

In some embodiments, the compounds of Formula E are those where R¹ is halo. In other embodiments, the compounds of Formula E are those where R¹ is fluoro. In other embodiments, the compounds of Formula E are those where R³ is halo or methyl. In other embodiments, the compounds of Formula D are those where R¹ is halo and R³ is halo or methyl.

In other embodiments, the invention provides methods for treating or preventing cancer, comprising administering to a subject an effective amount of a compound of Formula F

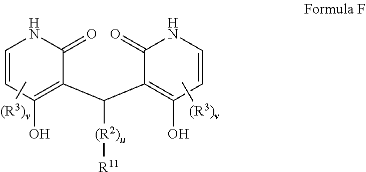

or a pharmaceutically acceptable salt or tautomer thereof, wherein R², R³, v, u, and R¹¹ are as provided above in the summary of the invention for the compounds or pharmaceutically acceptable salts of Formula F. In one embodiment, the subject is in need of treatment or prevention of cancer.

In certain embodiments, the compounds of Formula F are those where R¹¹ is

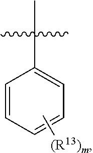

wherein each R¹³ is independently chloro, bromo, iodo, C₁-C₈ alkoxy, amino, hydroxy, cyano, C₁-C₈ alkyl, NHAc, or trihalomethyl and m is 3.

In some embodiments, the compounds of Formula F are those where u is 0; R³ is halo or methyl; and R¹¹ is

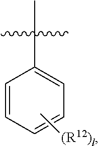

wherein each R¹² is halo.

Illustrative examples of the compounds of Formula F include the following:

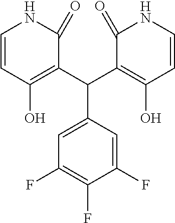 [SMILES: O=c1[nH]ccc(O)c1C(c1cc(F)c(F)c(F)c1)c1c(O)cc[nH]c1=O]

and/or its tautomer

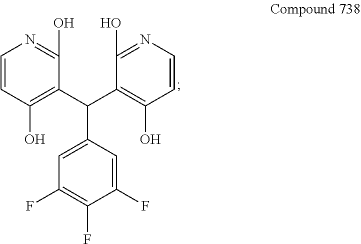 [SMILES: Oc1ccnc(O)c1C(c1cc(F)c(F)c(F)c1)c1c(O)ccnc1O]

In other embodiments, the invention provides methods for treating or preventing cancer, comprising administering to a subject an effective amount of a compound of the formula:

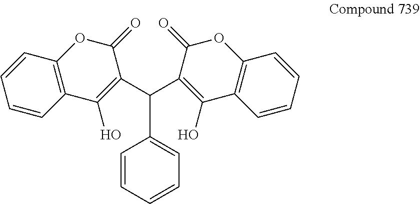 [SMILES: O=c1oc2ccccc2c(O)c1C(c1ccccc1)c1c(O)c2ccccc2oc1=O]

or a pharmaceutically acceptable salt thereof

2. Combination Therapy

In one aspect, the present methods for treating or preventing cancer can further comprise the administration of another anticancer agent.

In one embodiment, the present invention provides methods for treating or preventing cancer, comprising the administration of an effective amount of a Coumarin-Based Compound and another anticancer agent to a subject in need thereof. The Coumarin-Based Compound and another anticancer agent can be administered concurrently. In this embodiment, the Coumarin-Based Compound and another anticancer agent can be administered within the same composition, or can be administered from different compositions, via the same or different routes of administration. In another embodiment, the Coumarin-Based Compound is administered during a time when the other anticancer agent exerts its prophylactic or therapeutic effect, or vice versa.

In another embodiment, the Coumarin-Based Compound or other anticancer agent is administered in doses commonly employed when such agents are used as monotherapy for the treatment of cancer.

In one embodiment, the Coumarin-Based Compound or other anticancer agent is administered in doses that are lower than the doses commonly employed when such agents are used as monotherapy for the treatment of cancer.

In another embodiment, the Coumarin-Based Compound and other anticancer agent act synergistically and are administered in doses that are lower than the doses commonly employed when such agents are used as monotherapy for the treatment of cancer. The dosage of the Coumarin-Based Compound or other anticancer agent administered as well as the dosing schedule can depend on various parameters, including, but not limited to, the cancer being treated, the subject's general health, and the administering physician's discretion. A Coumarin-Based Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent, to a subject in need thereof. In various embodiments a Coumarin-Based Compound and the other anticancer agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, a Coumarin-Based Compound and the other anticancer agent are administered within 3 hours. In another embodiment, a Coumarin-Based Compound and the other anticancer agent are administered at 1 minute to 24 hours apart.

In one embodiment, an effective amount of a Coumarin-Based Compound and an effective amount of other anticancer agent are present in the same composition. In one embodiment, this composition is useful for oral administration, in another embodiment, this composition is useful for intravenous administration.

In one embodiment, the compositions comprise an amount of a Coumarin-Based Compound and the other anticancer agent which together are effective to treat or prevent cancer.

In another embodiment, the compositions comprise an effective amount of temozolomide, procarbazine, dacarbazine, interleukin-2, irinotecan, or doxorubicin, a pharmaceutically acceptable carrier or vehicle, and an effective amount of a Coumarin-Based Compound.

In one embodiment, the amount of a Coumarin-Based Compound and the other anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of combined amount of a Coumarin-Based Compound and the other anticancer agent by weight of the composition. Other compositions of the present invention are prepared so that a parenteral dosage unit contains from about 0.01% to about 2% by weight of the composition.

Cancers that can be treated or prevented by administering a Coumarin-Based Compound and the other anticancer agent include, but are not limited to, the list of cancers set forth above in Table 27.

In one embodiment, the cancer is brain cancer. In specific embodiments, the brain cancer is pilocytic astrocytoma, astrocytoma, anaplastic astrocytoma, glioblastoma multiforme or a metastatic brain tumor.

In one embodiment, the cancer is melanoma. In a specific embodiment, the melanoma is metastatic melanoma.

The Coumarin-Based Compound and other anticancer agent can act additively or synergistically. A synergistic combination of a Coumarin-Based Compound and the other anticancer agent, might allow the use of lower dosages of one or both of these agents and/or less frequent administration of the agents to a subject with cancer. The ability to utilize lower dosages of one or both of the Coumarin-Based Compound and other anticancer agent and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

In one embodiment, the administration of an effective amount of a Coumarin-Based Compound and an effective amount of another anticancer agent inhibits the resistance of a cancer to the other anticancer agent. In one embodiment, the cancer is a tumor.

Suitable other anticancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the other anticancer agent is, but is not limited to, a drug listed in Table 28.

TABLE 28
Alkylating agents, including but not limited to:

| | | |
|---|---|---|
| Nitrogen mustards: | Cyclophosphamide | Trofosfamide |
| | Ifosfamide | Chlorambucil |
| Nitrosoureas: | Carmustine (BCNU) | Lomustine (CCNU) |
| Alkylsulfonates: | Busulfan | Treosulfan |
| Triazenes: | Dacarbazine | Temozolomide |
| | Procarbazine | |
| Platinum containing | Cisplatin | Aroplatin |
| complexes: | Carboplatin | Oxaliplatin |

Plant alkaloids, including but not limited to:

| | | |
|---|---|---|
| Vinca alkaloids: | Vincristine | Vindesine |
| | Vinblastine | Vinorelbine |
| Taxoids: | Paclitaxel | Docetaxel |

DNA topoisomerase inhibitors, including but not limited to:

| | | |
|---|---|---|
| Epipodophyllins: | Etoposide | 9-aminocamptothecin |
| | Teniposide | Camptothecin |
| | Topotecan | Crisnatol |
| Mitomycins: | Mitomycin C | Anti-metabolites |

Anti-folates, including but not limited to:

| | | |
|---|---|---|
| DHFR inhibitors: | Methotrexate | Trimetrexate |
| IMP dehydrogenase | Mycophenolic acid | EICAR |
| inhibitors: | Tiazofurin | Ribavirin |
| Ribomiclotide | Deferoxamine | hydroxyurea |
| reductase inhibitors: | | |

Pyrimidine analogs, including but not limited to:

| | | |
|---|---|---|
| Uracil analogs: | 5-Fluorouracil | Doxifluridine |
| | Fluoxuridine | Ralitrexed |
| Cytosine analogs: | Cytarabine (ara C) | Gemcitabine |
| | Cytosine arabinoside | Capecitabine |
| | Fludarabine | |
| Purine analogs: | Mercaptopurine | Thioguanine |
| DNA anti-metabolites: | 3-HP | beta-TGDR |
| | 2′-deoxy-5- | cyclocytidine |
| | fluorouridine | |
| | 5-HP | guanazole |
| | alpha-TGDR | inosine glycodialdehyde |
| | aphidicolin | macebecin II |
| | glycinate | |
| | ara-C | Pyrazoloimidazole |
| | 5-aza-2′- | |
| | deoxycytidine | |

Hormonal therapies, including but not limited to:

| | | |
|---|---|---|
| Receptor | | |
| antagonists: | | |
| Anti-estrogen: | Tamoxifen | Megestrol |
| | Raloxifene | |
| LHRH agonists: | Goscrclin | Leuprolide acetate |
| Anti-androgens: | Flutamide | Bicalutamide |

Retinoids/deltoids, including but not limited to:

| | | |
|---|---|---|
| | Cis-retinoic acid | |
| Vitamin A | All-trans retinoic | |
| derivative: | acid (ATRA-IV) | |
| Vitamin D3 analogs: | EB 1089 | KH 1060 |
| | CB 1093 | |

Photodvnamic therapies, including but not limited to:

| | |
|---|---|
| Vertoporfm | Demethoxy-hypocrellin |
| (BPD-MA) | A |
| Plithalocyanine | (2BA-2-DMHA) |
| Photosensitizer Pc4 | |

Cytokines, including but not limited to:

| | |
|---|---|
| Interferon-α | Tumor necrosis factor |
| Interferon-β | Interleukin-2 |
| Interferon-γ | |

Angiogenesis inhibitors, including but not limited to:

| | |
|---|---|
| Angiostatin (plas- | MoAb IMC-ICl 1 |
| minogen fragment) | |
| antiangiogenic | Neovastat |
| antithrombin III | |
| Angiozyme | NM-3 |
| ABT-627 | Panzem |
| Bay 12-9566 | PI-88 |
| Benefin | Placental ribonuclease |
| | inhibitor |
| Bevacizumab | Plasminogen activator |
| | inhibitor |
| BMS-275291 | Platelet factor-4 (PF4) |
| cartilage-derived | Prinomastat |
| inhibitor (CDI) | |
| CAI | Prolactin 16 kD fragment |
| CD59 complement | Proliferin-related |
| fragment | protein (PRP) |
| CEP-7055 | PTK 787/ZK 222594 |
| Col 3 | Retinoids |
| Combretastatin A-4 | Solimastat |
| Endostatin (collagen | Squalamine |
| XVIII fragment) | |
| Fibronectin fragment | SS 3304 |
| Gro-beta | SU 5416 |
| Halofuginone | SU 6668 |
| Heparinases | SUl 1248 |
| Heparin hexa- | Tetrahydrocortisol-S |
| saccharide fragment | |
| HMV833 | Tetrathiomolybdate |
| Human chorionic | Thalidomide |
| gonadotropin (hCG) | |
| IM-862 | Thrombospondin-1 |
| | (TSP-I) |
| Interferon α/β/γ | TNP-470 |
| Interferon inducible | Transforming growth |
| protein (IP-10) | factor-beta (TGF-β) |
| Interleukin-12 | Vasculostatin |
| Kringle 5 (plas- | Vasostatin (cal- |
| minogen fragment) | reticulin fragment) |
| Marimastat | ZD6126 |
| Metalloproteinase | ZD 6474 |
| inhibitors (TIMPs) | |
| 2-Methoxyestradiol | farnesyl transferase |
| | inhibitors (FTI) |
| MMI 270 | Bisphosphonates |
| (CGS 27023A) | |

Antimitotic agents, including but not limited to:

| | |
|---|---|
| Allocolchicine | Maytansine |
| Halichondrin B | Rhizoxin |
| Colchicine | Thiocolchicine |
| colchicine derivative | trityl cysteine |
| dolstatin 10 | |

Others:

| | | |
|---|---|---|
| Isoprenylation | | |
| inhibitors: | | |
| Dopaminergic | l-methyl-4-phenyl- | |
| neurotoxins: | pyridinium ion | |
| Cell cycle | Staurosporine | |
| inhibitors: | | |
| Actinomycins: | Actinomycin D | Dactinomycin |
| Bleomycins: | Bleomycin A2 | Peplomycin |
| | Bleomycin B2 | |
| Anthracyclines: | Daunorubicin | Pirarabicin |
| | Doxorubicin | Zorabicin |
| | (adriamycin) | |
| | Idarubicin | Mitoxantrone |
| | Epirubicin | |
| MDR inhibitors: | Verapamil | |
| Ca²⁺ATPase | Thapsigargin | |
| inhibitors: | | |

Other additional anticancer agents that are useful in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefmgol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine hydrochloride; hydroxyurea; idarubicin hydrochloride; ifosfamide; ilmofosine; interleukin-2 (including recombinant interleukin-2, or rIL2), interferon alfa-2α; interferon alfa-2β; interferon alfa-n1; interferon alfa-n3; interferon beta-Iα; interferon gamma-Iβ; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamyciii; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Further anticancer drugs that are useful in the methods and compositions of the invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta Lactam Derivatives; beta-alethine; betaclamycin B; betulinic acid; bFGF inhibitor; bicalutamide; bisantrene; bisaziridinylspermme; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin Analogue; conagenin; crambescidin 816; crisnatol; cryptopliycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemniii B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine Analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin Analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drag resistance gene inhibitor; multiple tumor suppressor 1-based. therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel Analogues; paclitaxel derivatives; palauamiiie; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone Bl; raboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfm; temozolomide; teniposide; tetrachlorodecaoxide; tetrazomine; thalib lastine; thiocoraline; thrombopoietin; thrombopoietin mimetic; thymalfasin; thymopoietin receptor agonist; thymotrinan; thyroid stimulating hormone; tin ethyl etiopurpurirt; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; ver amine; verdins; verteporfm; vinorelbine; vinxaltine; vitaxin; vorozole; zanoterone; zeniplatin; zilascorb; and zinostatin stimalamer.

In another embodiment, the other anticancer agent is interferon-α. In another embodiment, the other anticancer agent is interleukin-2. In one embodiment, the other anticancer agent is an alkylating agent, such as a nitrogen mustard, a nitrosourea, an alkylsulfonate, a triazene, or a platinum-containing agent. In one embodiment, the other anticancer agent is a triazene alkylating agent In one embodiment, the other anticancer agent is O-6-benzylguanine. In another embodiment, the other anticancer agent is O-6-benzylguanine and temozolomide. In another embodiment, the other anticancer agent is O-6-benzylguanine and procarbazine. In still another embodiment, the other anticancer agent is O-6-benzylguanine and dacarbazine.

The Coumarin-Based Compounds can be administered to a subject that has undergone or is currently undergoing one or more additional anticancer therapies including, but not limited to, surgery, radiation therapy, or immunotherapy, such as cancer vaccines.

In one embodiment, the invention provides methods for treating or preventing cancer comprising administering to a subject in need thereof an effective amount of a Coumarin-Based Compound to treat or prevent cancer and another anticancer therapy including, but not limited to, surgery, radiation therapy, or immunotherapy, such as a cancer vaccine.

In one embodiment, the other anticancer therapy is radiation therapy. In another embodiment, the other anticancer therapy is surgery. In still another embodiment, the other anticancer therapy is immunotherapy.

In a specific embodiment, the present methods for treating or preventing cancer comprise administering an effective amount of a Coumarin-Based Compound and radiation therapy. The radiation therapy can be administered concurrently with, prior to, or subsequent to the Coumarin-Based Compound, in one embodiment at least an hour, five hours, 12 hours, a day, a week, a month, in another embodiment several months (e.g., up to three months), prior or subsequent to administration of the Coumarin-Based Compound. Where the other anticancer therapy is radiation therapy, any radiation therapy protocol can be administered depending upon the type of cancer to be treated. For example, but not by way of limitation, X-ray radiation can be administered; specifically, high-energy megavoltage (radiation of greater that 1 MeV energy) can be administered for deep tumors, and electron beam and orthovoltage X-ray radiation can be administered for skin cancers. Gamma-ray emitting radioisotopes, such as radioactive isotopes of radium, cobalt and other elements, can also be administered.

Additionally, the invention provides methods of treatment of cancer comprising administering a Coumarin-Based Compound as an alternative to chemotherapy or radiation therapy where the chemotherapy or the radiation therapy results in a negative side effect in the subject being treated. The subject being treated can, optionally, be treated with another anticancer therapy such as surgery, radiation therapy, or immunotherapy.

The Coumarin-Based Compounds can also be administered in vitro or ex vivo, such as for the treatment of certain cancers, including, but not limited to leukemias and lymphomas, such treatment involving autologous stem cell transplants. This can involve a process in which the subject's autologous hematopoietic stem cells are harvested and purged of all cancer cells, the subject's remaining bone-marrow cell population is then eradicated via the administration of a Coumarin-Based Compound and/or radiation, and the resultant stem cells are infused back into the subject. Supportive care can be subsequently provided while bone marrow function is restored and the subject recovers.

B. Treatment or Prevention of a Neurodegenerative Disease

The Coumarin-Based Compounds are useful for treating or preventing a neurodegenerative disease.

Accordingly, the invention provides methods for treating or preventing a neurodegenerative disease, comprising administering an effective amount of a Coumarin-Based Compound to a subject in need thereof. Examples of neurodegenerative diseases include, but are not limited to, Alexander's disease, Alper's disease, Alzheimer's disease, Amyotrophic lateral sclerosis, Ataxia telangiectasia. Batten disease (also known as Spielmeyer-Vogt-Sjogren-Batten disease), Bovine spongiform encephalopathy, Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Progressive Supranuclear Palsy, Refsum's disease, Sandhoffs disease, Schilder's disease, Subacute combined degeneration of spinal cord secondary to Pernicious Anaemia, Spinocerebellar ataxia, Spinal muscular atrophy, Steele-Richardson-Olszewski disease, and Tabes dorsalis. In one embodiment, the neurodegenerative disease is Alzheimer's disease. Other examples of neurdegenerative diseases include, but are not limited to, diffuse Lewy body disease, multisystem degeneration (Shy-Drager syndrome), motor neuron diseases including amyotrophic lateral sclerosis, degenerative ataxias, cortical basal degeneration, ALS-Parkinson's-Dementia complex of Guam, subacute sclerosing panencephalitis, Huntington's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, prion diseases (including Creutzfeldt-Jakob, Gerstmann-Straussler-Scheinker disease, Kuru and fatal familial insomnia), age-related dementia and other conditions with memory loss, such as vascular dementia, diffuse white matter disease (Binswanger's disease), dementia of endocrine or metabolic origin, dementia of head trauma and diffuse brain damage, dementia pugilistica and frontal lobe dementia, cerebral ischemia or infaction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

1. Coumarin-Based Compounds Useful for Treatment or Prevention of a Neurodegenerative Disease

In one embodiment, Coumarin-Based Compounds that are useful for treating or preventing a neurodegenerative disease are those of Formulas I to XXVI, set forth above.

In another embodiment, the invention emcompasses methods for treating or preventing cancer, comprising administering to a subject an effective amount of a compound of Formula A, B, C, D, E, or F, described above, or a pharmaceutically acceptable salt thereof. In one embodiment, the subject is in need of treatment or prevention of the neurodegenerative disease.

2. Combination Therapy

In one aspect, the present methods for treating or preventing a neurodegenerative disease can further comprise the administration of another anti-neurodegenerative disease agent.

In one embodiment, the present invention provides methods for treating or preventing a neurodegenerative disease, comprising the administration of an effective amount of a Coumarin-Based Compound and another anti-neurodegenerative disease agent to a subject in need thereof. The Coumarin-Based Compound and another anti-neurodegenerative disease agent can be administered concurrently. In this embodiment, the Coumarin-Based Compound and another anti-neurodegenerative disease agent can be administered within the same composition, or can be administered from different compositions, via the same or different routes of administration. In another embodiment, the Coumarin-Based Compound is administered during a time when the other anti-neurodegenerative disease agent exerts its prophylactic or therapeutic effect, or vice versa.

In another embodiment, the Coumarin-Based Compound or other anti-neurodegenerative disease agent is administered in doses commonly employed when such agents are used as monotherapy for the treatment of a neurodegenerative disease.

In one embodiment, the Coumarin-Based Compound or other anti-neurodegenerative disease agent is administered in doses that are lower than the doses commonly employed when such agents are used as monotherapy for the treatment of a neurodegenerative disease.

In another embodiment, the Coumarin-Based Compound and other anti-neurodegenerative disease agent act synergistically and are administered in doses that are lower than the doses commonly employed when such agents are used as monotherapy for the treatment of a neurodegenerative disease. The dosage of the Coumarin-Based Compound or other anti-neurodegenerative disease agent administered as well as the dosing schedule can depend on various parameters, including, but not limited to, the neurodegenerative disease being treated, the subject's general health, and the administering physician's discretion. A Coumarin-Based Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anti-neurodegenerative disease agent, to a subject in need thereof. In various embodiments a Coumarin-Based Compound and the other anti-neurodegenerative disease agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, a Coumarin-Based Compound and the other anti-neurodegenerative disease agent are administered within 3 hours. In another embodiment, a Coumarin-Based Compound and the other anti-neurodegenerative disease agent are administered at 1 minute to 24 hours apart.

In one embodiment, an effective amount of a Coumarin-Based Compound and an effective amount of other anti-neurodegenerative disease agent are present in the same composition. In one embodiment, this composition is useful for oral administration, in another embodiment, this composition is useful for intravenous administration.

In one embodiment, the compositions comprise an amount of a Coumarin-Based Compound and the other anti-neurodegenerative disease agent which together are effective to treat or prevent a neurodegenerative disease.

The Coumarin-Based Compound and other anti-neurodegenerative disease agent can act additively or synergistically. A synergistic combination of a Coumarin-Based Compound and the other anti-neurodegenerative disease agent, might allow the use of lower dosages of one or both of these agents and/or less frequent administration of the agents to a subject with a neurodegenerative disease. The ability to utilize lower dosages of one or both of the Coumarin-Based Compound and other anti-neurodegenerative disease agent and/or to administer the agents less frequently can reduce any toxicity associated with the administration of the agents to a subject without reducing the efficacy of the agents in the treatment of a neurodegenerative disease. In addition, a synergistic effect might result in the improved efficacy of these agents in the treatment of a neurodegenerative disease and/or the reduction of any adverse or unwanted side effects associated with the use of either agent alone.

In one embodiment, the administration of an effective amount of a Coumarin-Based Compound and an effective amount of another anti-neurodegenerative disease agent inhibits the resistance of a neurodegenerative disease to the other anti-neurodegenerative disease agent.

Suitable other anti-neurodegenerative disease agents useful in the methods and compositions of the present invention include, but are not limited to, anti-Alzheimer's agents such as cholinesterase inhibitors (e.g., tacrine, donepezil hydrochloride, rivastigmine, or galantamine), or partial glutamate antagonists (e.g., memantine), or anti-Parkinson's agents such as levodopa, carbidopa, tolcapone, bromocriptine, pergolide, pramipexole, ropinirole, selegiline, or amantadine.

C. Additional Combination Therapies

Additional agents that can be used in a combination product with Coumarin-Based Compounds for the treatment or prevention of diseases associated with γ-secretase activity or prevention of diseases associated with γ-secretase activity include, but are not limited to, a small molecule, a synthetic drug, a peptide (including a cyclic peptide), a polypeptide, a protein, a nucleic acid (e.g., a DNA and RNA nucleotide including, but not limited to, an antisense nucleotide sequence, a triple helix, RNAi, and a nucleotide sequence encoding a biologically active protein, polypeptide or peptide), an antibody, a synthetic or natural inorganic molecule, a mimetic agent, and a synthetic or natural organic molecule. Specific examples of such agents include, but are not limited to, an immunomodulatory agent (e.g., interferon), anti-inflammatory agent (e.g., an adrenocorticoid, a corticosteroid (e.g., beclomethasone, budesonide, flunisolide, fluticasone, triamcinolone, methylprednisolone, prednisolone, prednisone, hydrocortisone), a glucocorticoid, a steroid, and a non-steriodal anti-inflammatory drug (e.g., aspirin, ibuprofen, diclofenac, and a COX-2 inhibitor), a pain reliever, a leukotreine antagonist (e.g., montelukast, a methyl xanthine, zafirlukast, and zileuton), a beta2-agonist (e.g., albuterol, biterol, fenoterol, isoetharie, metaproterenol, pirbuterol, salbutamol, terbutalin formoterol, salmeterol, and salbutamol terbutaline), an anticholinergic agent (e.g., ipratropium bromide and oxitropium bromide), sulphasalazine, penicillamine, dapsone, an antihistamine, an anti-malarial agent (e.g., hydroxychloroquine), an anti-viral agent (e.g., a nucleoside analog (e.g., zidovudine, acyclovir, gangcyclovir, vidarabine, idoxuridine, trifluridine, and ribavirin), foscarnet, amantadine, rimantadine, saquinavir, indinavir, ritonavir, and AZT) and an antibiotic (e.g., dactinomycin (formerly actinomycin), bleomycin, erythomycin, penicillin, mithramycin, and anthramycin (AMC)).

Any therapy which is known to be useful, or which has been used, will be used or is currently being used for the treatment or prevention of diseases associated with γ-secretase activity can be used in combination with the Coumarin-Based Compounds in accordance with the invention described herein.

### V. Therapeutic or Prophylactic Administration and Compositions of the Invention

Due to their activity, Coumarin-Based Compounds are advantageously useful in veterinary and human medicine. As described above, the Coumarin-Based Compounds are useful for treating or preventing a Condition in a subject in need thereof. Without being bound by theory, it is believed that the Coumarin-Based Compounds exert their therapeutic or prophylactic effect by inhibiting γ-secretase.

The Coumarin-Based Compounds can be administered in amounts that are effective to treat or prevent a Condition in a subject, including a subject that is in need of treatment or prevention of a Condition.

When administered to a subject, the Coumarin-Based Compounds can be administered as a component of a composition that comprises a pharmaceutically acceptable carrier or vehicle. The pharmaceutically acceptable “carrier or vehicle” includes, for example, a diluent and an excipient. The present compositions, which comprise a Coumarin-Based Compound, can be administered orally. The Coumarin-Based Compounds can also be administered by any other convenient route, for example, by infusion or bolus injection, by absorption through epithelial or mucocutaneous linings (e.g., oral, rectal, or intestinal mucosa) and can be administered together with another biologically active agent. Administration can be systemic or local. Various delivery systems are known, e.g., encapsulation in liposomes, microparticles, microcapsules and capsules.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, specifically to the ears, nose, eyes, or skin. In some instances, administration will result in the release of a Coumarin-Based Compound into the bloodstream.

In one embodiment, the Coumarin-Based Compounds are administered orally. In other embodiments, it can be desirable to administer the Coumarin-Based Compounds locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Coumarin-Based Compounds into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the Coumarin-Based Compounds can be formulated as a suppository, with traditional binders and excipients such as triglycerides.

In another embodiment Coumarin-Based Compounds can be delivered in a vesicle, specifically a liposome (see Langer, Science 249:1527-1533 (1990) and Liposomes in Therapy of Infectious Disease and Cancer 317-327 and 353-365 (1989)).

In yet another embodiment, the Coumarin-Based Compounds can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249: 1527-1533 (1990) can be used. In one embodiment a pump can be used (Langer, Science 249: 1527-1533 (1990); Sefton, CRC Crit. Ref. Biomed. Eng. 14:201 (1987); Buchwald et al, Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sd. Rev. Macromol. Chem. 2:61 (1983); Levy et al, Science 228:190 (1935); During et al, Ann. Neural. 25:351 (1989); and Howard et al, J. Neurosurg. 71:105 (1989)).

In yet another embodiment a controlled- or sustained-release system can be placed in proximity of a target of the Coumarin-Based Compounds, e.g., the spinal column, brain, skin, lung, or gastrointestinal tract, thus requiring only a fraction of the systemic dose.

The present compositions can optionally comprise a suitable amount of a pharmaceutically acceptable excipient so as to provide the form for proper administration to the subject.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when the Coumarin-Based Compound is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule (see e.g. U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment, the Coumarin-Based Compound is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a Coumarin-Based Compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the Coumarin-Based Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection.

Generally, the ingredients are supplied either separately or mixed together in unit dosage form, for example, as a dry lyophilized-powder or water-free concentrate in a hermetically sealed container such as an ampule or sachette indicating the quantity of active agent. Where the Coumarin-Based Compounds are to be administered by infusion, they can be dispensed, for example, with an infusion bottle containing sterile pharmaceutical grade water or saline. Where the Coumarin-Based Compounds are administered by injection, an ampule of sterile water for injection or saline can be provided so that the ingredients can be mixed prior to administration.

Coumarin-Based Compounds can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus provides single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Coumarin-Based Compound to treat or prevent the Condition over a period of time. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the Coumarin-Based Compound, and can thus reduce the occurrence of adverse side effects. Controlled- or sustained-release compositions can initially release an amount of a Coumarin-Based Compound that promptly produces the desired therapeutic or prophylactic effect, and gradually and continually release other amounts of the Coumarin-Based Compound to maintain this level of therapeutic or prophylactic effect over an extended period of time. To maintain a constant level of the Coumarin-Based Compound in the body, the Coumarin-Based Compound can be released from the dosage form at a rate that will replace the amount of Coumarin-Based Compound being metabolized and excreted from the body.

Controlled- or sustained-release of an active ingredient can be stimulated by various conditions, including but not limited to, changes in pH, changes in temperature, concentration or availability of enzymes, concentration or availability of water, or other physiological conditions or compounds. The amount of the Coumarin-Based Compounds that is effective in the treatment or prevention of a Condition can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. Suitable effective dosage amounts, however, range from about 10 micrograms to about 5 grams about every 4 hours, although they are typically about 500 mg or less per every 4 hours. In one embodiment, the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Coumarin-Based Compound is administered, the effective dosage amounts correspond to the total amount administered.

Compositions can be prepared according to conventional mixing, granulating or coating methods, respectively, and the present compositions can contain, in one embodiment, from about 0.1% to about 99%; and in another embodiment from about 1% to about 70% of the Coumarin-Based Compound by weight or volume.

The dosage regimen utilizing the Coumarin-Based Compound can be selected in accordance with a variety of factors including type, species, age, weight, sex and medical condition of the subject; the severity of the condition to be treated; the route of administration; the renal or hepatic function of the subject; and the specific Coumarin-Based Compound employed. A person skilled in the art can readily determine the effective amount of the drug useful for treating or preventing the Condition. An Coumarin-Based Compound can be administered in a single daily dose, or the total daily dosage can be administered in divided doses of two, three or four times daily. Furthermore, a Coumarin-Based Compound can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of Coumarin-Based Compound ranges from about 0.1% to about 15%, w/w or w/v. The Coumarin-Based Compounds can be assayed in vitro or in vivo for the desired therapeutic or prophylactic activity prior to use in humans. Animal model systems can be used to demonstrate safety and efficacy.

In certain embodiments, a Coumarin-Based Compound or pharmaceutical composition thereof is administered to a human that has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In some embodiments, a Coumarin-Based Compound or pharmaceutical composition thereof is administered to a human infant. In other embodiments, a Coumarin-Based Compound or pharmaceutical composition thereof is administered to a human toddler. In other embodiments, a Coumarin-Based Compound or pharmaceutical composition thereof is administered to a human child. In other embodiments, a Coumarin-Based Compound or pharmaceutical composition thereof is administered to a human adult. In yet other embodiments, a Coumarin-Based Compound or pharmaceutical composition thereof is administered to an elderly human.

In certain embodiments, a Coumarin-Based Compound or pharmaceutical composition thereof is administered a subject in an immunocompromised state or immunosuppressed state or at risk for becoming immunocompromised or immunosuppressed. In certain embodiments, a Coumarin-Based Compound or pharmaceutical composition thereof is administered to a subject receiving or recovering from immunosuppressive therapy.

In some embodiments, a Coumarin-Based Compound or pharmaceutical composition thereof is administered to a patient who is susceptible to adverse reactions to conventional anti-γ-secretase therapies. In some embodiments, a γ-secretase inhibitor or pharmaceutical composition thereof is administered to a patient who has proven refractory to anti-γ-secretase therapies other than γ-secretase inhibitors, but are no longer on these therapies. Among these patients are refractory patients, and patients who are too young for conventional therapies.

In some embodiments, the subject being administered a Coumarin-Based Compound or pharmaceutical composition thereof has not received therapy prior to the administration of the Coumarin-Based Compound or pharmaceutical composition thereof.

### VI. Kits Comprising a Coumarin-Based Compound

The invention provides kits that can simplify the administration of a Coumarin-Based Compound to a subject.

A typical kit of the invention comprises a unit dosage form of a Coumarin-Based Compound. In one embodiment, the unit dosage form is a container, which can be sterile, containing an effective amount of a Coumarin-Based Compound and a pharmaceutically acceptable carrier or vehicle. The kit can further comprise a label or printed instructions instructing the use of the Coumarin-Based Compound to treat or prevent a Condition. The kit can also further comprise a unit dosage form of another prophylactic or therapeutic agent, for example, a container containing an effective amount of the other prophylactic or therapeutic agent. In one embodiment, the kit comprises a container containing an effective amount of a Coumarin-Based Compound and an effective amount of another prophylactic or therapeutic agent. Examples of other prophylactic or therapeutic agents include, but are not limited to, those listed above.

Having described the invention with reference to certain embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### General Procedure for the Synthesis of Coumarin-Based Compounds of Formulas I to IV, VI, VII, IX to XI, and XIII

4-Hydroxycoumarin or 4-hydroxy-6-methylcoumarin (3 mmol) is dissolved in 6 ml of hot ethanol. The corresponding aldehyde (1.5 mmol) is then added to the solution and the resultant mixture is refluxed for about 18 hours. The mixture is then cooled to room temperature and the resultant solid is collected from the mixture by filtration. The collected solid is then crystallized to provide the desired Coumarin-Based Compound.

### Example 2

### Synthesis of a Substituted Coumarin

This example provides a synthesis of 6-fluorocoumarin, which can be used as a starting material for preparing compounds provided herein. With slight modifications to the protocol provided below, coumarins with other substituents can be prepared.

A mixture of 4-fluorophenol (1.4 g, 12.5 mmol), malonic acid (1.5 g, 14.4 mmol), anhydrous zinc chloride (5.0 g, 37.5 mmol), and phosphorus oxychloride (4 ml) was heated with stirring at 60° C. for 48 h. The mixture was then cooled, and ice and water were added to the mixture. The resultant crude product was extracted from the mixture with CH₂Cl₂ (3×10 ml). The combined CH₂Cl₂ extracts were washed with brine and dried over Na₂SO₄. The solvent was then evaporated to provide a residue. The residue was purified using chromatography on silica gel (CH₂Cl₂/acetone 9:1) to provide 6-fluorocoumarin (156.2 mg, 7%) as a yellow solid.

### Example 3

### Synthesis of Compound 203

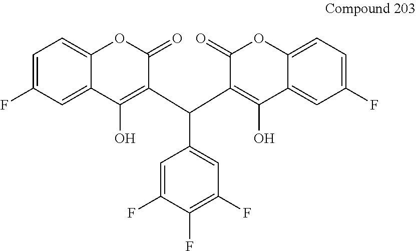 [SMILES: O=c1oc2ccc(F)cc2c(O)c1C(c1cc(F)c(F)c(F)c1)c1c(O)c2cc(F)ccc2oc1=O]

To a solution of 6-fluoro-4-hydroxycoumarin (50 mg, 0.28 mmol) in hot ethanol (2.0 mL), was added 3,4,5-trifluorobenzaldehyde (0.015 mL, 0.14 mmol). The resulting mixture was refluxed at 85° C. for 24 h, and cooled to room temperature. The solid was filtered off, washed with ethanol to give the product 203 (28 mg, 40%).

### Example 4

### Synthesis of Compound 53

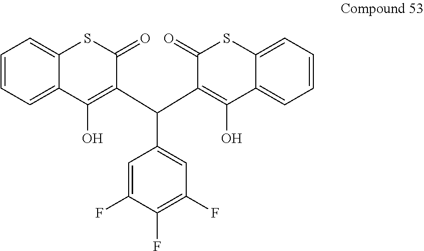 [SMILES: O=c1sc2ccccc2c(O)c1C(c1cc(F)c(F)c(F)c1)c1c(O)c2ccccc2sc1=O]

To a solution of thiosalicylic acid (1.0 g, 6.5 mmol) in tetrahydrofuran (33 ml) was added methyllithium (26 mmol, 16 ml of 1.6 M solution in ether) at 0° C. The resultant reaction mixture was stirred for 18 hours at room temperature. The reaction mixture was then quenched with water, followed by a saturated NH₄Cl solution. The organic phase was separated and the aqueous phase was extracted with EtOAc (3×50 ml). The combined organic extracts were dried over Na₂SO₄, and the solvent was evaporated to provide an oil residue. The oil residue was purified using chromatography on silica gel (hexane/EtOAc 9:1) to provide o-mercaptoacetophenone (885 mg, 90%) as a yellow oil.

Sodium hydride (1.0 g, 26.3 mmol of a 60% dispersion in oil) was slowly added to a solution of o-mercaptoacetophenone (400 mg, 2.6 mmol) and diethyl carbonate (0.9 ml) in toluene (7.0 ml). The mixture was refluxed for 4 hours, and then stirred at room temperature for 18 hours. Water (20 ml) was then added to the mixture. The mixture was then acidified with 1N HCl and extracted with CH₂Cl₂ (3×20 ml). The organic layers were dried over Na₂SO₄, and the solvent was evaporated. The crude product was purified using chromatography on silica gel (hexane/EtOAc 6:4) to provide 4-hydroxy-2H-thiochromen-2-one (45.6 mg, 10%) as a white solid.

To a solution of 4-hydroxy-2H-thiochromen-2-one (45 mg, 0.25 mmol) in ethanol (2.0 mL) was added 3,4,5-trifluorobenzaldehyde (0.014 mL, 0.13 mmol). The resulting mixture was refluxed at 85° C. for 24 h, and cooled to room temperature. The solid was filtered off, washed with ethanol to give the product 53 (6.7 mg, 11%).

### Example 5

### Synthesis of Compound 735

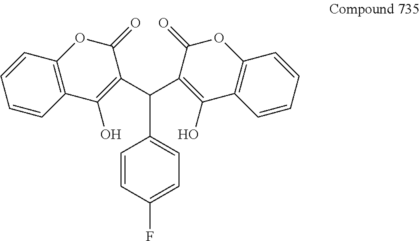 [SMILES: O=c1oc2ccccc2c(O)c1C(c1ccc(F)cc1)c1c(O)c2ccccc2oc1=O]

To a solution of 4-hydroxycoumarin (500 mg, 3.00 mmol) in ethanol (6.0 mL), was added 4-fluorobenzaldehyde (0.16 mL, 1.50 mmol). The resulting mixture was refluxed at 85° C. for 24 h, and cooled to room temperature. The solid was filtered off, washed with ethanol to give the product 735 (578.3 mg, 90%).

### Example 6

### Synthesis of Compound 737

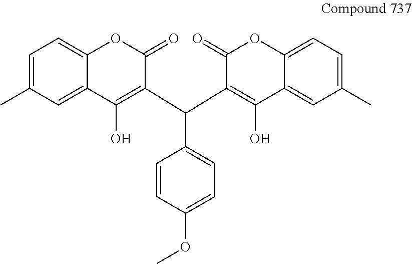

To a solution of 4-hydroxy-6-methylcoumarin (500 mg, 2.84 mmol) in ethanol (6.0 mL), was added 4-methoxybenzaldehyde (0.17 mL, 1.42 mmol). The resulting mixture was refluxed at 85° C. for 24 h, and cooled to room temperature. The solid was filtered off, washed with ethanol to give the product 737 (478.4 mg, 72%).

### Example 7

### Synthesis of Compound 37

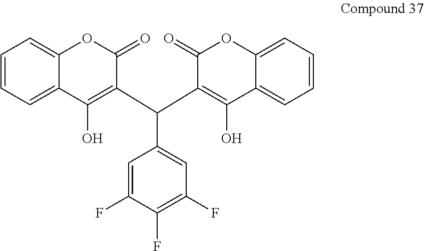 [SMILES: O=c1oc2ccccc2c(O)c1C(c1cc(F)c(F)c(F)c1)c1c(O)c2ccccc2oc1=O]

To a solution of 4-hydroxycoumarin (500 mg, 3.00 mmol) in ethanol (6.0 mL), was added 3,4,5-trifluorobenzaldehyde (0.17 mL, 1.50 mmol). The resulting mixture was refluxed at 85° C. for 24 h, and cooled to room temperature. The solid was filtered off, washed with ethanol to give the product 37 (520.0 mg, 72%).

Another synthesis for the preparation of compound 37 was performed as follows: To an ice cold solution of formic acid (1.38 ml, 37.00 mmol) was added triethylamine dropwise (1.68 ml, 12.00 mmol). The solution was kept at this temperature until the smoke disappeared, at which point 3,4,5-trifluorobenzaldehyde (0.35 mL, 3.00 mmol) and 4-hydroxycoumarin (500 mg, 3.00 mmol) were added sequentially. The mixture was refluxed at 130° C. for 4 h, and then cooled to room temperature. The reaction mixture was diluted with H₂O (6.0 mL), extracted with ethyl acetate (50.0 mL), dried over Na₂SO₄ and concentrated under vacuo. The crude product was recrystallized in ethanol to give the product 37 (412.3 mg, 45%).

### Example 8

### Synthesis of Compound 423

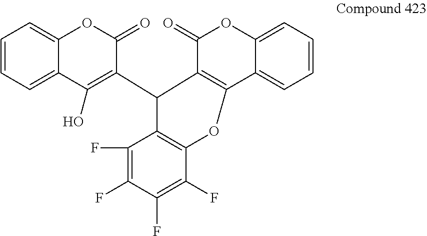 [SMILES: O=c1oc2ccccc2c(O)c1C1c2c(F)c(F)c(F)c(F)c2Oc2c1c(=O)oc1ccccc21]

To a solution of 4-hydroxycoumarin (500 mg, 3.00 mmol) in ethanol (3.0 mL), was added a solution of pentafluorobenzaldehyde (0.19 mL, 1.54 mmol) in ethanol (2.0 mL). The resulting mixture was refluxed at 85° C. for 24 h, and cooled to room temperature. The solid was filtered off, washed with ethanol to give the product 423 (268.9 mg, 37%).

### Example 9

### Synthesis of Compound 369

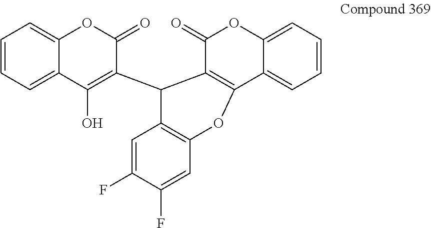 [SMILES: O=c1oc2ccccc2c(O)c1C1c2cc(F)c(F)cc2Oc2c1c(=O)oc1ccccc21]

To a solution of 4-hydroxycoumarin (500 mg, 3.00 mmol) in ethanol (6.0 mL), was added 2,4,5-trifluorobenzaldehyde (0.18 mL, 1.50 mmol). The resulting mixture was refluxed at 85° C. for 48 h, and cooled to room temperature. The solid was filtered off, washed with ethanol to give the product 369 (104 mg, 16%).

### Example 10

### Synthesis of Compound 209

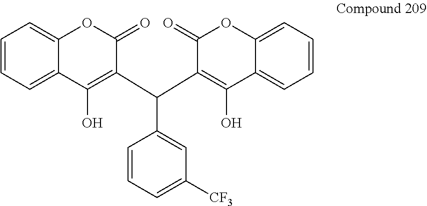 [SMILES: O=c1oc2ccccc2c(O)c1C(c1cccc(C(F)(F)F)c1)c1c(O)c2ccccc2oc1=O]

To a solution of 4-hydroxycoumarin (500 mg, 3.00 mmol) in ethanol (6.0 mL), was added 3-(trifluoromethyl)benzaldehyde (0.21 mL, 1.50 mmol). The resulting mixture was refluxed at 85° C. for 24 h, and cooled to room temperature. The solid was filtered off, washed with ethanol to give the product 209 (582.6 mg, 79%).

### Example 11

### Synthesis of Compound 728

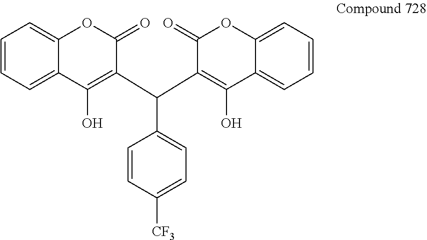 [SMILES: O=c1oc2ccccc2c(O)c1C(c1ccc(C(F)(F)F)cc1)c1c(O)c2ccccc2oc1=O]

To a solution of 4-hydroxycoumarin (500 mg, 3.00 mmol) in ethanol (3.0 mL), was added a solution of 4-(trifluoromethyl)benzaldehyde (0.21 mL, 1.50 mmol) in ethanol (1.0 mL). The resulting mixture was refluxed at 85° C. for 24 h, and cooled to room temperature. The solid was filtered off, washed with ethanol to give the product 728 (474.6 mg, 66%).

### Example 12

### Synthesis of Compound 257

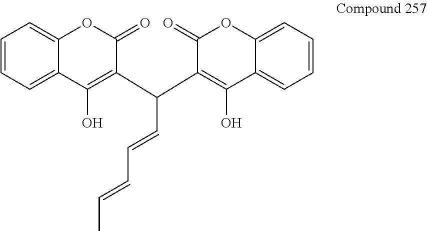 [SMILES: C/C=C/C=C/C(c1c(O)c2ccccc2oc1=O)c1c(O)c2ccccc2oc1=O]

To a solution of 4-hydroxycoumarin (500 mg, 3.00 mmol) in ethanol (9.0 mL), was added 2,4-hexadienal (0.17 mL, 1.50 mmol). The resulting mixture was refluxed at 85° C. for 24 h, and cooled to room temperature. The solid was filtered off, washed with ethanol to give the product 257 (17.6 mg, 3%).

### Example 13

### Synthesis of Compound 736

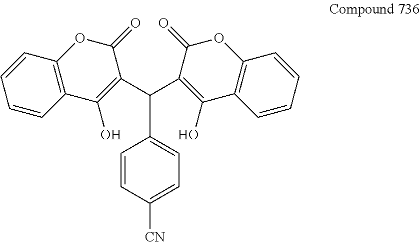 [SMILES: N#Cc1ccc(C(c2c(O)c3ccccc3oc2=O)c2c(O)c3ccccc3oc2=O)cc1]

To a solution of 4-hydroxycoumarin (500 mg, 3.00 mmol) in ethanol (3.0 mL), was added a solution of 4-cyanobenzaldehyde (202 mg, 1.50 mmol) in ethanol (6.0 mL). The resulting mixture was refluxed at 85° C. for 24 h, and cooled to room temperature. The solid was filtered off, washed with ethanol to give the product 736 (391.2 mg, 60%).

### Example 14

### Synthesis of Compound 77

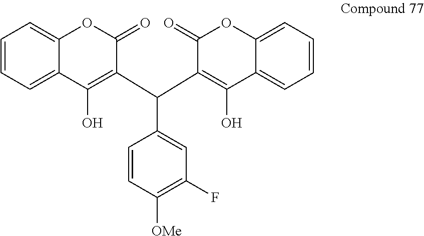 [SMILES: COc1ccc(C(c2c(O)c3ccccc3oc2=O)c2c(O)c3ccccc3oc2=O)cc1F]

To a solution of 4-hydroxycoumarin (500 mg, 3.00 mmol) in ethanol (3.0 mL), was added a solution of 3-fluoro-4-methoxybenzaldehyde (237 mg, 1.50 mmol) in ethanol (6.0 mL). The resulting mixture was refluxed at 85° C. for 24 h, and cooled to room temperature. The solid was filtered off, washed with ethanol to give the product 77 (651.2 mg, 94%).

### Example 15

### Synthesis of Compound 732

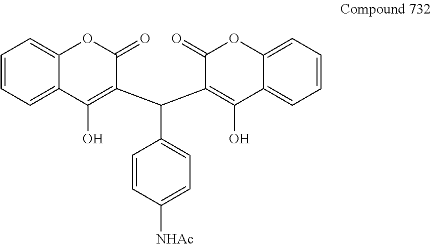 [SMILES: CC(=O)Nc1ccc(C(c2c(O)c3ccccc3oc2=O)c2c(O)c3ccccc3oc2=O)cc1]

To a solution of 4-hydroxycoumarin (500 mg, 3.00 mmol) in ethanol (3.0 mL), was added a solution of 4-acetamidobenzaldehyde (251 mg, 1.50 mmol) in ethanol (9.0 mL). The resulting mixture was refluxed at 85° C. for 24 h, and cooled to room temperature. The solid was filtered off, washed with ethanol to give the product 732 (285.2 mg, 40%).

### Example 16

### Synthesis of Compound 733

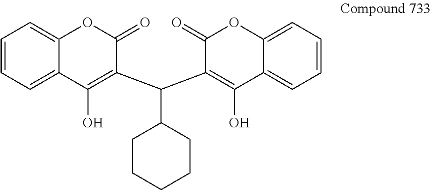 [SMILES: O=c1oc2ccccc2c(O)c1C(c1c(O)c2ccccc2oc1=O)C1CCCCC1]

To a solution of 4-hydroxycoumarin (500 mg, 3.00 mmol) in ethanol (9.0 mL), was added cyclohexanecarboxaldehyde (0.19 mL, 1.50 mmol). The resulting mixture was refluxed at 85° C. for 72 h, and cooled to room temperature. The solid was filtered off, washed with ethanol to give the product 733 (74.6 mg, 12%).

### Example 17

### Synthesis of Compound 42

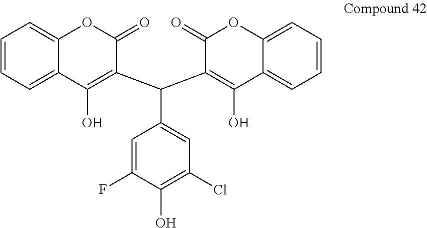 [SMILES: O=c1oc2ccccc2c(O)c1C(c1cc(F)c(O)c(Cl)c1)c1c(O)c2ccccc2oc1=O]

To a solution of 4-hydroxycoumarin (500 mg, 3.00 mmol) in ethanol (9.0 mL), was added 3-chloro-5-fluoro-4-hydroxybenzaldehyde (269 mg, 1.50 mmol). The resulting mixture was refluxed at 85° C. for 24 h, and cooled to room temperature. The solid was filtered off, washed with ethanol to give the product 42 (428.5 mg, 59%).

### Example 18

### Synthesis of Compound 372

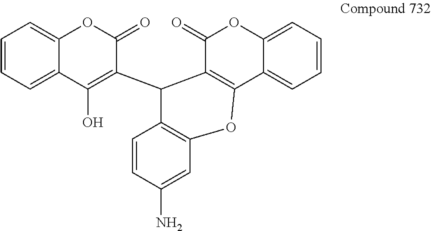 [SMILES: Nc1ccc2c(c1)Oc1c(c(=O)oc3ccccc13)C2c1c(O)c2ccccc2oc1=O]

To a solution of 4-hydroxycoumarin (94 mg, 0.58 mmol) in ethanol (5.0 mL), was added 4-amino-2-chloro-benzaldehyde (45 mg, 0.29 mmol). The resulting mixture was refluxed at 85° C. for 24 h, and cooled to room temperature. The solid was filtered off, washed with ethanol to give the product 732 (40 mg, 32%).

### Example 19

### Synthesis of Compound 210

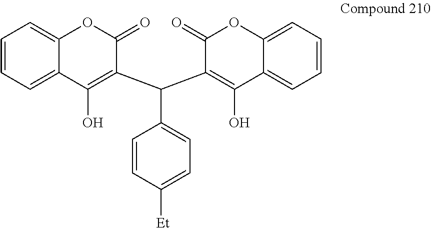 [SMILES: CCc1ccc(C(c2c(O)c3ccccc3oc2=O)c2c(O)c3ccccc3oc2=O)cc1]

To a solution of 4-hydroxycoumarin (500 mg, 3.00 mmol) in ethanol (5.0 mL), was added 4-ethylbenzaldehyde (0.21 mL, 1.50 mmol). The resulting mixture was refluxed at 85° C. for 24 h, and cooled to room temperature. The solid was filtered off, washed with ethanol to give the product 210 (625.1 mg, 95%).

### Example 20

### Synthesis of Compound 1

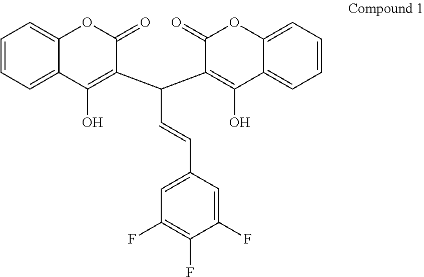 [SMILES: O=c1oc2ccccc2c(O)c1C(/C=C/c1cc(F)c(F)c(F)c1)c1c(O)c2ccccc2oc1=O]

To a solution of 4-hydroxycoumarin (26 mg, 0.16 mmol) in ethanol (0.5 mL), was added 3,4,5-triflourocinnamicaldehyde (15 mg, 0.08 mmol, prepared from reduction of 3,4,5-triflourocinnamic acid). The resulting mixture was refluxed at 90° C. for 24 h, and cooled to room temperature. The solid was filtered off, washed with ethanol to give the product 1 (3.8 mg, 10%).

### Example 21

### Synthesis of Compound 45

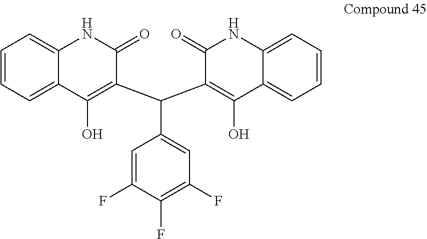 [SMILES: O=c1[nH]c2ccccc2c(O)c1C(c1cc(F)c(F)c(F)c1)c1c(O)c2ccccc2[nH]c1=O]

To a solution of 2,4-quinolinediol (500 mg, 3.10 mmol) in ethanol (19.0 mL), was added 3,4,5-trifluorobenzaldehyde (0.18 mL, 1.55 mmol). The resulting mixture was refluxed at 90° C. for 24 h, and cooled to room temperature. The solid was filtered off, washed with ethanol to give the product 45 (588 mg, 82%).

### Example 22

### Synthesis of Compound 61

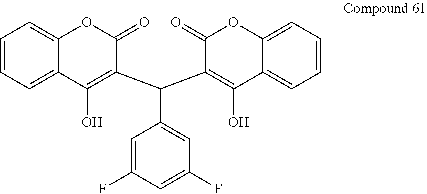 [SMILES: O=c1oc2ccccc2c(O)c1C(c1cc(F)cc(F)c1)c1c(O)c2ccccc2oc1=O]

To a solution of 4-hydroxycoumarin (500 mg, 3.00 mmol) in ethanol (9.0 mL), was added 3,5-difluorobenzaldehyde (0.17 mL, 1.50 mmol). The resulting mixture was refluxed at 90° C. for 24 h, and cooled to room temperature. The solid was filtered off, washed with ethanol to give the product 61 (397.6 mg, 29%).

### Example 23

### Synthesis of Compound 735

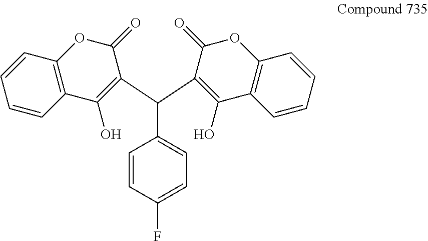 [SMILES: O=c1oc2ccccc2c(O)c1C(c1ccc(F)cc1)c1c(O)c2ccccc2oc1=O]

To a solution of 4-hydroxycoumarin (500 mg, 3.00 mmol) in ethanol (6.0 mL), was added 4-fluorobenzaldehyde (0.16 mL, 1.50 mmol). The resulting mixture was refluxed at 85° C. for 24 h, and cooled to room temperature. The solid was filtered off, washed with ethanol to give the product 735 (578.3 mg, 90%).

### Example 24

### Synthesis of Compound 197

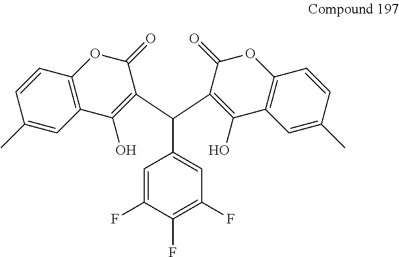

To a solution of 4-hydroxy-6-methylcoumarin (500 mg, 2.84 mmol) in ethanol (6.0 mL), was added 3,4,5-trifluorobenzaldehyde (0.16 mL, 1.42 mmol). The resulting mixture was refluxed at 85° C. for 24 h, and cooled to room temperature. The solid was filtered off, washed with ethanol to give the product 197 (320.4 mg, 46%).

### Example 25

### Synthesis of Compound 738

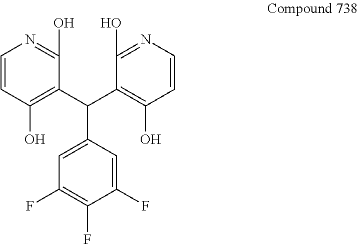 [SMILES: Oc1ccnc(O)c1C(c1cc(F)c(F)c(F)c1)c1c(O)ccnc1O]

To a solution of 2,4-dihydroxypyridine (250 mg, 2.25 mmol) in ethanol (5.0 mL), was added 3,4,5-trifluorobenzaldehyde (0.13 mL, 1.13 mmol). The resulting mixture was refluxed at 90° C. for 24 h, and cooled to room temperature. The solid was filtered off, washed with ethanol to give the product 738 (241.4 mg, 59%).

### Example 26

### Synthesis of Compound 734

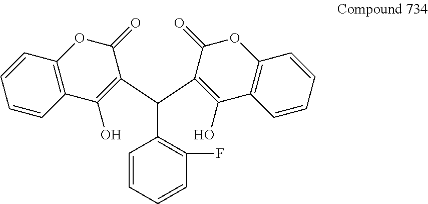 [SMILES: O=c1oc2ccccc2c(O)c1C(c1ccccc1F)c1c(O)c2ccccc2oc1=O]

To a solution of 4-hydroxycoumarin (500 mg, 3.00 mmol) in ethanol (9.0 mL), was added 2-fluorobenzaldehyde (0.16 mL, 1.50 mmol). The resulting mixture was refluxed at 90° C. for 24 h, and cooled to room temperature. The solid was filtered off, washed with ethanol to give the product 734 (329.4 mg, 25%).

### Example 27

### Synthesis of Compound 739

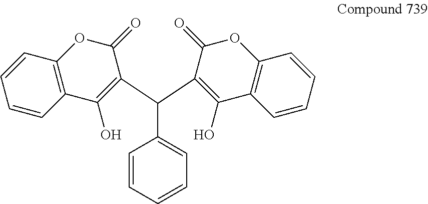 [SMILES: O=c1oc2ccccc2c(O)c1C(c1ccccc1)c1c(O)c2ccccc2oc1=O]

To a solution of 4-hydroxycoumarin (500 mg, 3.00 mmol) in ethanol (9.0 mL), was added benzaldehyde (0.16 mL, 1.50 mmol). The resulting mixture was refluxed at 90° C. for 24 h, and cooled to room temperature. The solid was filtered off, washed with ethanol to give the product 739 (530 mg, 86%).

### Example 28

### In Vitro Inhibition of γ-Secretase Activity

Without being bound by theory, it is believed that inhibiting γ-secretase, particularly that which generates Aβ42, or increasing the Aβ40/Aβ42 ratio, is desirable for the treatment or prevention of a Condition, particularly Alzheimer's disease.

Several of the above-described Coumarin-Based Compounds show in vitro inhibition of γ-secretase activity that generates Aβ40 and inhibition of γ-secretase activity that generates Aβ42. IC₅₀ values for inhibition of Aβ40 and Aβ42 were measured. The ratio of the IC₅₀ value for inhibition of γ-secretase activity that generates Aβ40 to the IC₅₀ value for inhibition of γ-secretase activity that generates Aβ42 was also calculated. The results are summarized below in Table 29.

The assay protocol employed was a modified version of that described in Li et al., 2000, Proc. Nat'l Acad. Sci. USA 97:6183-643, incorporated herein by reference. Briefly, recombinant peptide substrate was incubated with γ-secretase (40 μg/ml) in the presence or absence of test compound. The reaction mixture contained 0.25% CHAPSO, 0.1 μg/μl BSA, protease inhibitor, 50 mM PIPES, pH 7.0, 5 mM MgCl₂, 5 mM CaCl₂ and 150 mM KCl. The reaction was incubated for 2.5 hr at 37° C. and stopped by adding RIPA buffer (150 mM NaCl, 1.0% NP-40, 0.5% sodium deoxycholate, 0.1% SDS, 50 mM Tris HCl, pH 8.0). The products were detected with various antibody combinations using electrochemiluminescence (ECL) technology as previously described in Li et al., 2000, Proc. Nat'l Acad. Sci. USA 97:6183-643; Lai et al., 2003, J. Biol. Chem. 278: 22475-22481; and Yin et al., 2007, J. Biol. Chem. 282:23639-23644. The amount of product was determined using synthetic peptide or recombinant standards.

TABLE 29
In vitro inhibition of γ-secretase activity for Coumarin-Based Compounds

| | | IC₅₀ (μM) | | |
|---|---|---|---|---|
| | | | | Aβ40/Aβ42 |
| Cpd. | Chemical Structure | Aβ40 | Aβ42 | ratio |
| 209 | 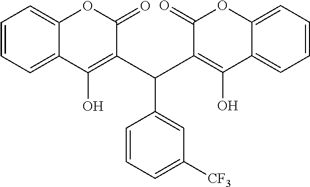 [SMILES: O=c1oc2ccccc2c(O)c1C(c1cccc(C(F)(F)F)c1)c1c(O)c2ccccc2oc1=O] | 2.0 | 0.8 | 2.5 |
| 77 | 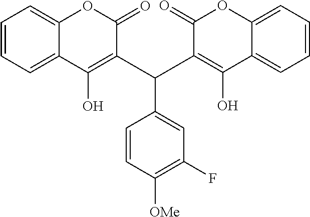 [SMILES: COc1ccc(C(c2c(O)c3ccccc3oc2=O)c2c(O)c3ccccc3oc2=O)cc1F] | 6.2 | 2.8 | 2.2 |
| 58 | 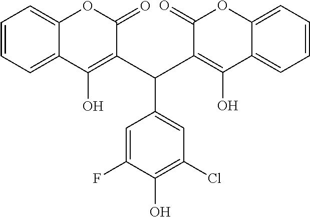 [SMILES: O=c1oc2ccccc2c(O)c1C(c1cc(F)c(O)c(Cl)c1)c1c(O)c2ccccc2oc1=O] | 4.8 | 3.1 | 1.5 |
| 210 | 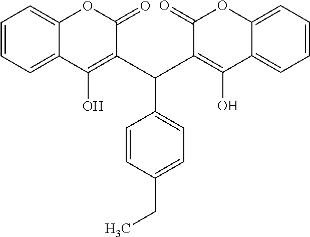 [SMILES: CCc1ccc(C(c2c(O)c3ccccc3oc2=O)c2c(O)c3ccccc3oc2=O)cc1] | 4.6 | 1.9 | 2.4 |
| 37 | 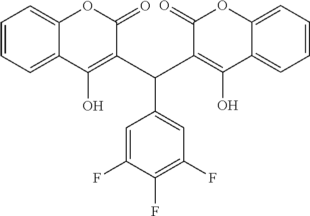 [SMILES: O=c1oc2ccccc2c(O)c1C(c1cc(F)c(F)c(F)c1)c1c(O)c2ccccc2oc1=O] | 0.6 | 0.2 | 3.0 |
| 423 | 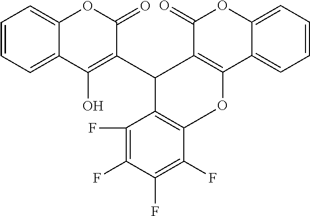 [SMILES: O=c1oc2ccccc2c(O)c1C1c2c(F)c(F)c(F)c(F)c2Oc2c1c(=O)oc1ccccc21] | 3.4 | 1.3 | 2.6 |
| 369 | 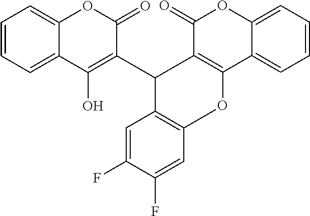 [SMILES: O=c1oc2ccccc2c(O)c1C1c2cc(F)c(F)cc2Oc2c1c(=O)oc1ccccc21] | 2.6 | 0.9 | 2.9 |
| 372 | 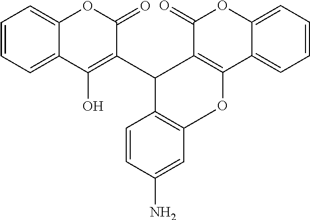 [SMILES: Nc1ccc2c(c1)Oc1c(c(=O)oc3ccccc13)C2c1c(O)c2ccccc2oc1=O] | 19.4 | 9.9 | 2.0 |
| 257 | 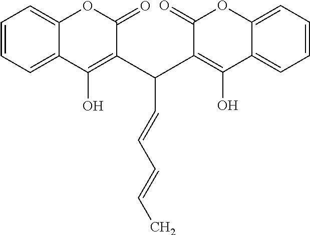 [SMILES: C/C=C/C=C/C(c1c(O)c2ccccc2oc1=O)c1c(O)c2ccccc2oc1=O] | 9.9 | 7.3 | 1.4 |
| (trans)- 258 | 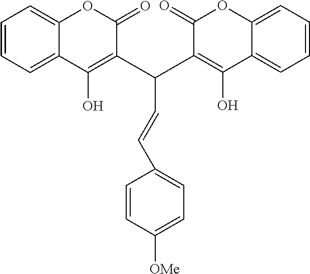 [SMILES: COc1ccc(/C=C/C(c2c(O)c3ccccc3oc2=O)c2c(O)c3ccccc3oc2=O)cc1] | 21.5 | 5.6 | 3.8 |
| (trans)- 1 | 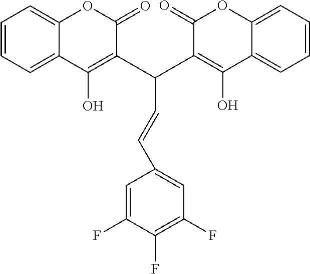 [SMILES: O=c1oc2ccccc2c(O)c1C(/C=C/c1cc(F)c(F)c(F)c1)c1c(O)c2ccccc2oc1=O] | 3.7 | 2.9 | 1.3 |
| 203 | 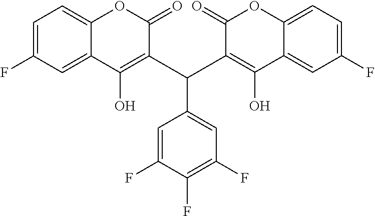 [SMILES: O=c1oc2ccc(F)cc2c(O)c1C(c1cc(F)c(F)c(F)c1)c1c(O)c2cc(F)ccc2oc1=O] | 2.6 | 0.9 | 2.9 |
| 199 | 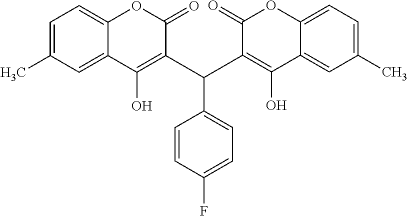 | 4.0 | 0.9 | 4.4 |
| 668 | 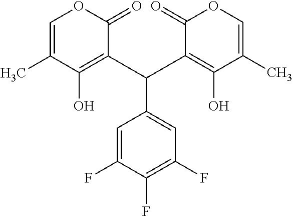 | 4.2 | 1.7 | 2.5 |
| 45 | 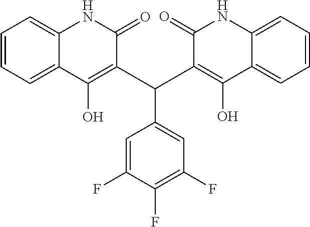 [SMILES: O=c1[nH]c2ccccc2c(O)c1C(c1cc(F)c(F)c(F)c1)c1c(O)c2ccccc2[nH]c1=O] | 8.0 | 3.4 | 2.4 |
| 53 | 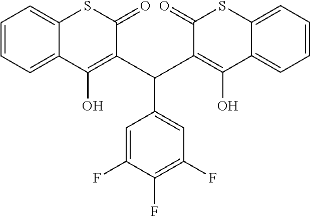 [SMILES: O=c1sc2ccccc2c(O)c1C(c1cc(F)c(F)c(F)c1)c1c(O)c2ccccc2sc1=O] | 1.3 | 0.5 | 2.6 |
| 520 | 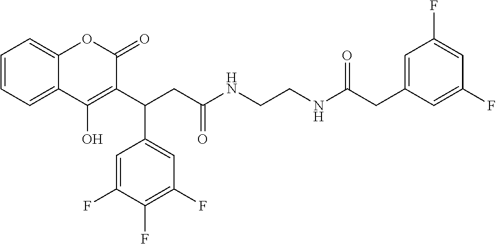 [SMILES: O=C(Cc1cc(F)cc(F)c1)NCCNC(=O)CC(c1cc(F)c(F)c(F)c1)c1c(O)c2ccccc2oc1=O] | 9.23 | 4.53 | 2.0 |
| 61 | 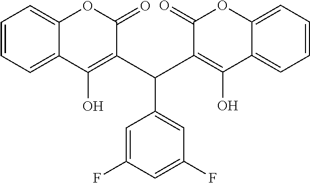 [SMILES: O=c1oc2ccccc2c(O)c1C(c1cc(F)cc(F)c1)c1c(O)c2ccccc2oc1=O] | 0.34 | 0.17 | 2.0 |
| 512 | 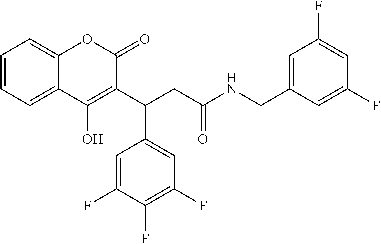 [SMILES: O=C(CC(c1cc(F)c(F)c(F)c1)c1c(O)c2ccccc2oc1=O)NCc1cc(F)cc(F)c1] | 1.86 | 1.24 | 1.5 |
| 197 | 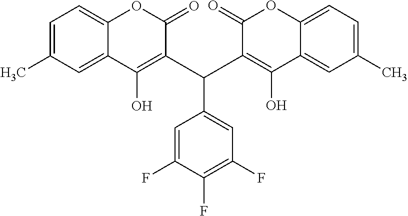 | 3.26 | 1.59 | 2.1 |
| 476 | 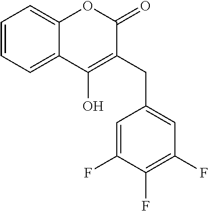 [SMILES: O=c1oc2ccccc2c(O)c1Cc1cc(F)c(F)c(F)c1] | ND (>100 μM) | ND (>100 μM) | n/a |
| 560 | 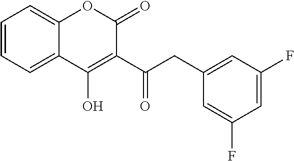 [SMILES: O=C(Cc1cc(F)cc(F)c1)c1c(O)c2ccccc2oc1=O] | 89.9 | 26.9 | 3.3 |
| 728 | 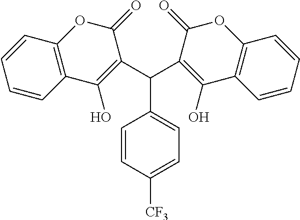 [SMILES: O=c1oc2ccccc2c(O)c1C(c1ccc(C(F)(F)F)cc1)c1c(O)c2ccccc2oc1=O] | 7.0 | 1.6 | 4.4 |
| 729 | 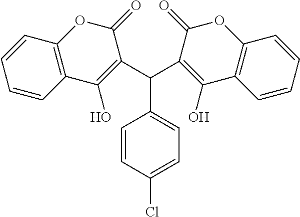 [SMILES: O=c1oc2ccccc2c(O)c1C(c1ccc(Cl)cc1)c1c(O)c2ccccc2oc1=O] | 1.7 | 1.2 | 1.4 |
| 738 | 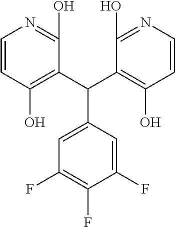 [SMILES: Oc1ccnc(O)c1C(c1cc(F)c(F)c(F)c1)c1c(O)ccnc1O] | ND (>100 μM) | ND (>100 μM) | n/a |
| 730 | 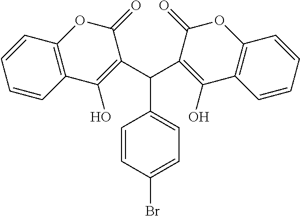 [SMILES: O=c1oc2ccccc2c(O)c1C(c1ccc(Br)cc1)c1c(O)c2ccccc2oc1=O] | 1.7 | 1.2 | 1.4 |
| 731 | 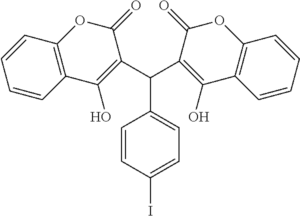 [SMILES: O=c1oc2ccccc2c(O)c1C(c1ccc(I)cc1)c1c(O)c2ccccc2oc1=O] | 6.1 | 1.5 | 4.1 |
| 732 | 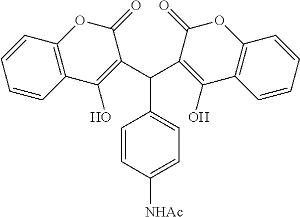 [SMILES: CC(=O)Nc1ccc(C(c2c(O)c3ccccc3oc2=O)c2c(O)c3ccccc3oc2=O)cc1] | 63.9 | 15.9 | 4.0 |
| 733 | 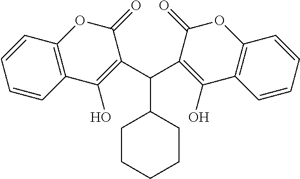 [SMILES: O=c1oc2ccccc2c(O)c1C(c1c(O)c2ccccc2oc1=O)C1CCCCC1] | 4.2 | 1.2 | 3.5 |
| 734 | 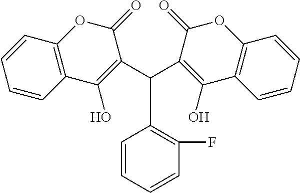 [SMILES: O=c1oc2ccccc2c(O)c1C(c1ccccc1F)c1c(O)c2ccccc2oc1=O] | 1.95 | 7.2 | 0.3 |

### Example 29

### Cell-based Assay for Production of Aβ38, Aβ40 and Aβ42 peptides

The following cell-based assay can be used for assessing inhibitory activity of test compounds on γ-secretase activity on APP expressed in stably transfected cells. Cells such as HEK239 or N2A cells that stably express APP are incubated 24-48 hr. in medium to which is added γ-secretase with or without test compound. The conditioned medium is collected. Secreted Aβ peptides are detected by electrochemiluminescence (ECL) technology as previously described, for example, in Li et al., 2000, Proc. Nat'l Acad. Sci. USA 97:6183-643; Lai et al., 2003, J. Biol. Chem. 278: 22475-22481; and Yin et al., 2007, J. Biol. Chem. 282:23639-23644. Concentration of Aβ peptides can be calculated from standard curves that are generated using synthetic peptides using the ECL assay.

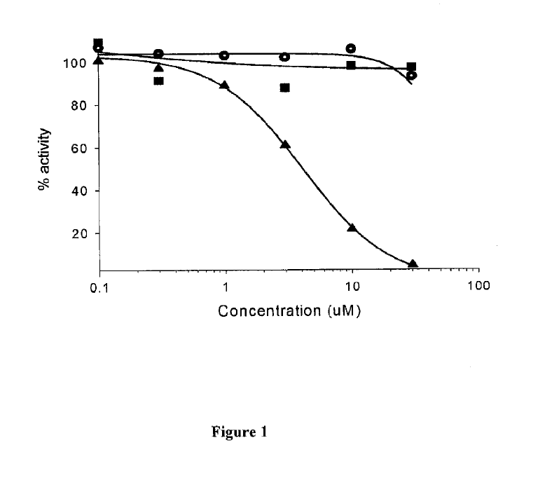

Results of a cell-based assay are provided in FIG. 1 in which cells stably transfected with APP were incubated in medium containing γ-secretase activity and the indicated amounts of compound 37. These results show that as concentrations of compound 37 are increased in the medium there is a decrease in the amount of Aβ42 (triangles) secreted from the cells. The amounts of Aβ38 (squares) and Aβ40 (circles) secreted remain relatively constant between cells treated with different concentrations of compound 37.

### Example 30

### Modulation of γ-Secretase Specificity Using Small Molecule Allosteric Inhibitors

### Abstract

γ-Secretase cleaves multiple substrates within the transmembrane domain that include the amyloid precursor protein as well as the Notch family of receptors. These substrates are associated with Alzheimer disease (AD) and cancer. Despite extensive investigation of this protease, little is known regarding the regulation of γ-secretase specificity. To discover selective inhibitors for drug development and for probing the mechanisms of γ-secretase specificity, we screened chemical libraries and consequently developed a di-coumarin family of inhibitors that preferentially inhibits γ-secretase-mediated production of Aβ42 over other cleavage activities. Provided coumarin-dimer based compounds interact with γ-secretase by binding to an allosteric site. By developing a multiple photoaffinity probe approach, we demonstrate that this allosteric binding causes a conformational change within the active site of γ-secretase at the S2 and S1 subsites that leads to selective inhibition of Aβ42. Utilizing these di-coumarin compounds, we reveal an unprecedented mechanism by which γ-secretase specificity is regulated and provide insights into the molecular basis by which familial presenilin mutations may affect the active site and specificity of γ-secretase. Furthermore, this class of selective inhibitors may be useful in medicine, and particularly in the development of AD therapeutics.

### Introduction

γ-Secretase is a multi-protein membrane-bound complex that is currently at the frontline of basic and translational research. It is composed of at least four proteins that include Presenilin, Nicastrin, Aph-1 and Pen-2 (1). Presenilin is believed to contain the active site of γ-secretase (2-4). It represents a novel class of protease that catalyzes peptide bond hydrolysis within the transmembrane hydrophobic environment and plays an essential role in a newly emerged signaling pathway known as regulated intramembrane proteolysis (5). γ-Secretase cleaves a variety of type I membrane proteins that include the amyloid precursor protein (APP) and the Notch family of proteins despite limited primary sequence homology across targeted substrates (6). Elucidation of the mechanisms that control the specificity of γ-secretase for these substrates has been hindered due to technical difficulties associated with intramembrane enzymology. Determining the factors that contribute to γ-secretase specificity is critical to understanding the biology of this unique protease and targeting it for therapeutic purposes.

γ-Secretase is an appealing drug target for Alzheimer disease and cancer. γ-Secretase cleaves APP to generate neurotoxic Aβ peptides, ranging from 37 to 46 amino acids in length (7). Among them, Aβ40 and Aβ42 have been extensively investigated for their association with AD (7). Additionally, disease-causing familial AD mutations (FAD) within APP, presenilin-1 (PS-1) and presenilin-2 (PS-2) proteins result in an increase in the ratio of Aβ42 to Aβ40 (see review (7)). Mutations in both enzyme and substrate can influence the specificity of γ-secretase and lead to pathological consequences. Non-selective inhibition of γ-secretase activity has been explored as an AD and cancer therapeutic approach, however the abrogation of all activities of γ-secretase results in toxicity in the gastrointestinal tract due to the blockage of Notch1 signaling (8). Therefore, the development of selective inhibitors is necessary to investigate γ-secretase specificity and provide candidates for drug development.

Recent studies have indicated that the ratio of Aβ42 to Aβ40, rather than the total amount of β-amyloid, correlates with the amount of characteristic AD plaques in mouse models (9-10) as well as with the age of onset of familial Alzheimer disease (11). Furthermore, new evidence suggests that Aβ40 may even play a neuroprotective role against AD progression whereas Aβ42 is more hydrophobic and more readily aggregates to form toxic oligomers and fibrils (10). As discussed herein, the discovery and development of selective γ-secretase inhibitors that specifically abrogate Aβ42 production over Aβ40 and Notch cleavage is a promising strategy for AD therapy.

Weggen et al. discovered that a subset of non-steroidal anti-inflammatory drugs, referred to as γ-secretase modulators (GSMs), were able to selectively decrease γ-secretase-mediated production of Aβ42 with a concomitant increase in Aβ38, and had no effect on Aβ40 or Notch1 cleavage (12). Conversely, other GSMs were determined to stimulate the production of Aβ42 while reducing Aβ38 cleavage. Subsequent studies have shown that these GSMs alter γ-secretase cleavage preference by binding directly to the APP substrate and not to γ-secretase (13). Other compounds that target γ-secretase and preferentially inhibit Aβ40 and Aβ42 production over Notch1 processing have been reported (14-15) although the precise action mechanism of these molecules has not been established. Therefore, it is critical to develop a better understanding of the molecular basis of γ-secretase specificity in order to facilitate the development of selective γ-secretase inhibitors (GSIs) for the treatment of AD and other human disorders.

In the present study, we describe a novel class of GSIs that contain a di-coumarin core and modulate γ-secretase specificity for Aβ42 production over Aβ38, Aβ40 and Notch cleavages. We have demonstrated that these inhibitors regulate γ-secretase activity by binding to an allosteric site within the γ-secretase complex. Furthermore, we have developed a multiple photoaffinity probe strategy using transition-state inhibitors that allows us to evaluate the architecture of the active site of γ-secretase. Using this method we demonstrate that the binding of di-coumarin compounds to γ-secretase causes a conformational change in the 51 and S2 subsites which may explain the selective regulation of protease by these small molecules. This work offers unprecedented evidence of a molecular mechanism by which γ-secretase specificity is modulated by small probes and could potentially explain how certain PS1 familial mutations influence AD. These inhibitors represent important tools that will help elucidate factors contributing to γ-secretase specificity and its relationship to AD, and represent an important contribution to AD therapy.

### Results

### Di-Coumarin Compounds are Selective γ-Secretase Inhibitors In Vitro

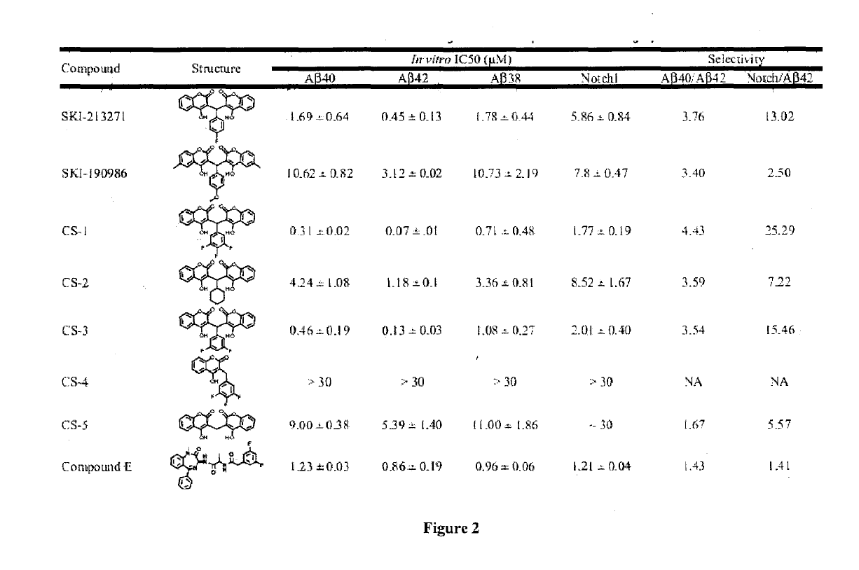

To discover selective GSIs, we screened large collections of small molecules (˜200,000 compounds) at the Sloan-Kettering Institute High Throughput Screening (HTS) Core Facility. Our HTS approach uncovered several novel classes of GSIs as well as currently established scaffolds. Among them, the presented class contains a symmetric di-coumarin core joined by a central benzene ring that displays specificity against Aβ42 production. The HTS screen revealed five inactive compounds in this structural class and two active hits: SKI-213271 and SKI-190986. In our multiple in vitro assays, both compounds selectively abrogated Aβ42 production over Aβ40 (FIG. 2) by approximately 3.5-fold. Additionally, we determined that both lead compounds did not promote Aβ38 production, which is distinct from the previously reported GSMs (12). Lastly, the coumarin-dimer compounds also exhibited decreased potency for inhibition of Notch-1 processing. Clearly, these compounds could represent a novel class of inhibitors that selectively target Aβ42 production. To develop more potent and selective inhibitors, we synthesized more than 40 analogs and have profiled a few in Table 1 with the respective IC₅₀ values for each in vitro assay listed. The predominant trend for this family of compounds was increased potency against Aβ42 over Aβ40, Aβ38, or Notch. The most effective compound, CS-1, exhibited in vitro IC₅₀ values of 0.07 μM, 0.31 μM, 0.71 μM, and 1.77 μM against Aβ42, Aβ40, Aβ38, and Notch respectively. The inactivity of CS-4 suggests that the coumarin-dimer structure is necessary for inhibitory potency. Conversely, Compound E, a potent pan-GSI, did not exhibit any significant selectivity for any of the cleavage activities assayed (FIG. 2). Preliminary structure-activity relationship analyses showed that the mono-, di- and tri-fluoro benzene ring incrementally increased the potency and selectivity of the compounds. Substitution of the fluorobenzene moiety with cyclohexane (CS-2) or hydrogen (CS-5) significantly reduced the potency and selectivity (FIG. 2). Furthermore, we tested the ability of CS-1 to retain its selectivity against γ-secretase from mouse brain membrane and found that it did maintain its preference for Aβ42 inhibition (IC₅₀'s: Aβ40=380 nM±35, Aβ42=112 nM±40). Lastly, we also determined the inhibitory potency of CS-1 against cell membrane prepared from cells that stably express the PS1-M146L familial mutation (16). The IC₅₀'s of CS-1 are 167±21 nM and 206±57 nM for Aβ40 and Aβ42, respectively.

### Di-Coumarin Compounds are Selective γ-Secretase Inhibitors in Cells

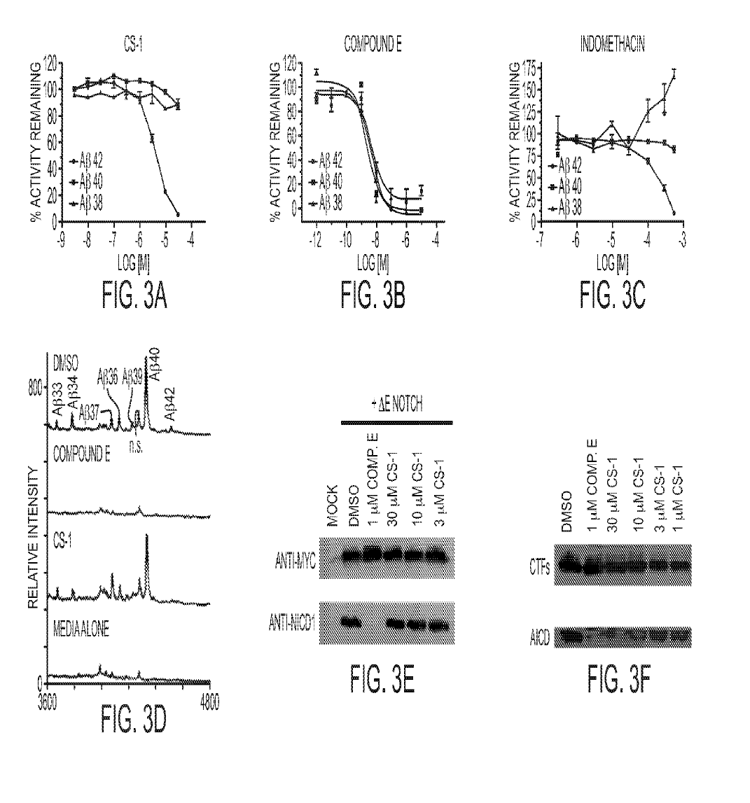

We next set out to determine if the selective inhibition of Aβ42 was maintained in a cell-based system for APP processing. First, we compared our lead compound CS-1 (FIG. 3a) to Compound E (FIG. 3b) and the GSM compound indomethacin (FIG. 3c). N2a mouse neuroblastoma cells that stably express Swedish-mutated APP substrate were treated with the indicated compounds for 24 hrs at 37° C. Following 24 hr incubation period, the medium was collected from the cells and assayed for secreted Aβ42, Aβ40, and Aβ38. CS-1 inhibited Aβ42 production with an EC₅₀ of approximately 3 μM in our cell-based assay, yet had virtually no effect on Aβ38 or Aβ40 production up to 30 μM (FIG. 3a). Furthermore, cytotoxicity studies using Alamar Blue indicated CS-1 had little to no effect on cell viability up to 30 μM (data not shown). In addition, we found that CS-3 exhibited an identical inhibitory profile with a slightly increased EC₅₀ for Aβ42 inhibition (˜5 μM). Compound E inhibited the production of all three β-amyloid species with equal potency (FIG. 3b), whereas indomethacin significantly enhanced Aβ38 production, abrogated Aβ42, and had no effect on Aβ40 (FIG. 3c). The result for indomethacin mirrored those findings by Kukar et al. whereby a different cell-based system was utilized (17), further validating our assay system for analysis of these Aβ species. We next confirmed these findings using immunoprecipitation-mass spectrometry (IP-MS) that revealed that CS-1 was able to inhibit Aβ42 while leaving Aβ38 and Aβ40 production largely intact (FIG. 3d). In a cell system, the coumarin-dimer based compounds retained their selectivity and exhibited an even greater specificity for inhibition of γ-secretase activity for Aβ42 production, which is a promising finding for drug development. This may reflect subtle variations between the cellular and in vitro conformations of γ-secretase. Nevertheless, the cell-based studies confirmed that CS-1 maintains a preference for inhibition of the γ-secretase mediated production of Aβ42 over Aβ40 or Aβ38, which is distinct to previously reported GSMs (17) and inhibitors (14-15, 18).

We next determined the ability of CS-1 to suppress cellular γ-secretase activity for Notch1 cleavage. The ΔE Notch construct encodes a truncated Notch1 protein that lacks the majority of the extracellular domain and no longer requires ligand binding or S2 cleavage (19). The fragment expressed by the ΔE Notch construct is a membrane-tethered portion of the Notch-1 receptor that is a direct substrate of γ-secretase. ΔE Notch was transiently expressed in HEK-293 cells for 24 hrs in the presence of DMSO or GSI. The expression of ΔE Notch protein was confirmed by anti-Myc antibody. We found that Compound E effectively blocked all production of the Notch intracellular domain (NICD) as detected by the anti-NICD1 SM320 antibody. However, CS-1 at concentrations up to 30 μM, which was able to abrogate virtually all of Aβ42 production, had no effect on NICD generation (FIG. 3e). In addition, we examined the potency of CSI-1 on AICD production and determined that it is less potent for this cleavage with an IC₅₀ >10 μM (FIG. 3f). This result further highlights the selectivity of this class of coumarin-dimer compound for Aβ42 inhibition.

### Di-Coumarin Inhibitors are Non-Competitive Inhibitors

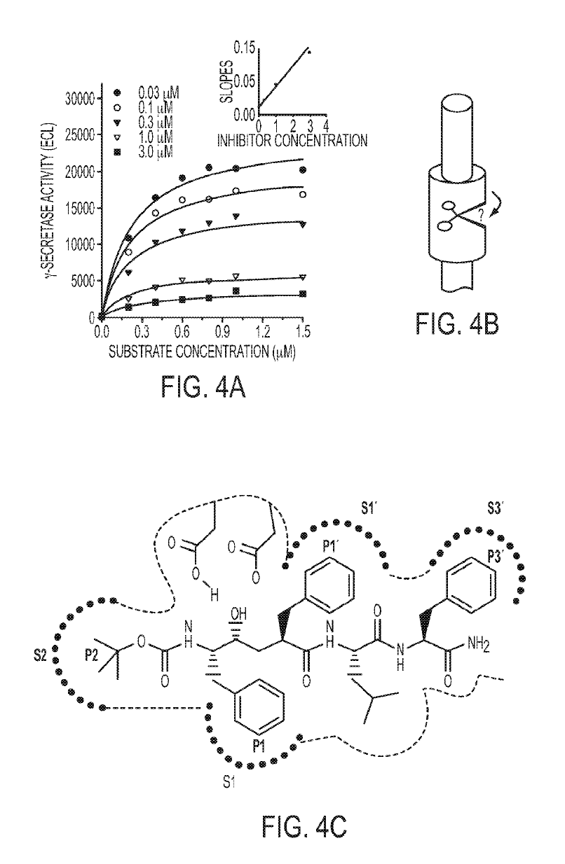

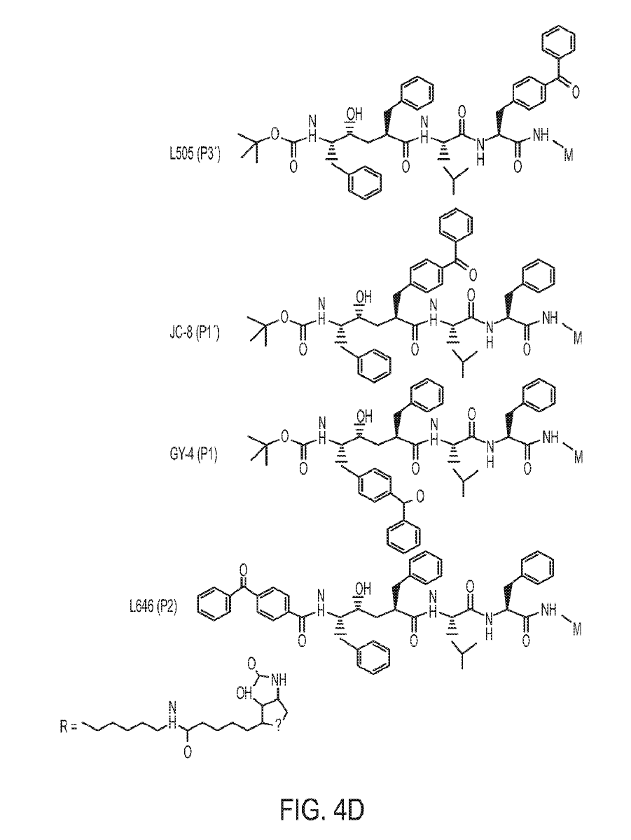

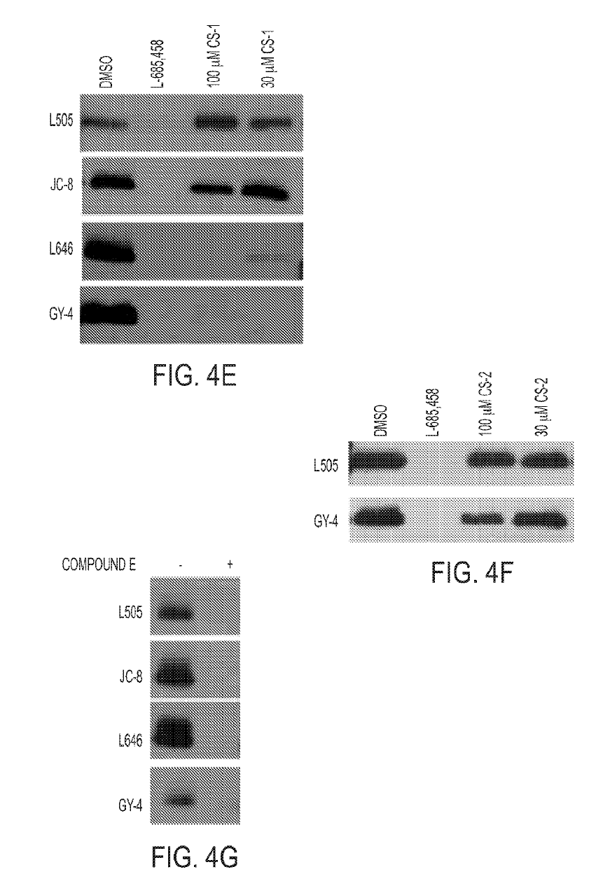

Following the realization that CS-1 and its analogs were exhibiting an in vitro and cell-based selectivity for Aβ42 over other γ-secretase cleavage activities, we examined their mechanism of action Inhibition kinetic analysis of CS-1 showed that it affects Vmax, but not Km indicating non-competitive inhibition against the APP-transmembrane domain substrate (APP-TM) (FIG. 4a), whereas L-685,458 (L458), a transition state inhibitor (20) behaves as a competitive inhibitor against the same substrate. The findings regarding L458 were consistent with our previous report (21). Additionally, the replotting of slope against inhibitor concentration shows a linear relationship (R²=0.98) (FIG. 4a, inlet), suggesting a purely non-competitive inhibition and a single inhibitor binding site. It is noteworthy to point out that L458 acts as a non-competitive inhibitor when the C100 substrate is used due to a putative docking site interaction (22). The non-competitive behavior of this class of inhibitors against APP-TM suggests that the coumarin dimer compounds are binding to γ-secretase at an allosteric site and thereby preventing enzyme activity.

### Di-Coumarin Inhibitors Alter the Subsites of the γ-Secretase Active Site

We hypothesized that the allosteric binding of the di-coumarin compounds alters the conformation of the active site of γ-secretase and thereby preferentially affects the Aβ42 site cleavage (FIG. 4b). This raised the technical issue of how to probe the contours of the enzymatic active site. Although the structure of γ-secretase has been determined by cryoelectron microscopy (23), the resolution attained is not sufficient to investigate subtle changes within the active site. Consequently, we developed a series of active-site directed inhibitors that incorporate a photoreactive benzophenone entity into varied positions. Using these photoreactive probes, we assessed the effect of the di-coumarin inhibitor binding on the active site of γ-secretase. Since the efficiency of photoinsertion depends on the orientation of the probe and the proximity of residues within the active site, conformational change of the active site can alter the orientation of the probe and contact residues and lead to altered cross-linking efficiencies. Therefore, multiple photoactivatable, active-site directed GSIs will provide a practical approach to evaluate the changes within the active site following allosteric di-coumarin binding.

L458 contains a hydroxyethylamine transition-state isostere that mimics the tetrahedral intermediate of aspartyl proteases and this moiety hydrogen bonds with the catalytic aspartate residues of γ-secretase (20). According to the nomenclature of Schechter and Berger (24), L458 contains the P2, P1, P1′, P2′ and P3′ residues that putatively bind to the S2, S1, S1′, S2′ and S3′ subsites, respectively, within the active site of γ-secretase (FIG. 4c). We have developed a series of biotinylated, photoactivatable inhibitors based on the core structure of L458 that allow us to probe the sub-pockets of the γ-secretase active site (3, 25-26). These inhibitors all have an individual benzophenone group incorporated into L458 at either the P2, P1, P1′, or P3′ position and are referred to as L646, GY4, JC8 and L505 (FIG. 4d). Each of these inhibitors interacts and labels the S2, S1, S1′, and S3′ subsites, respectively, within the γ-secretase complex (FIG. 4c-d).

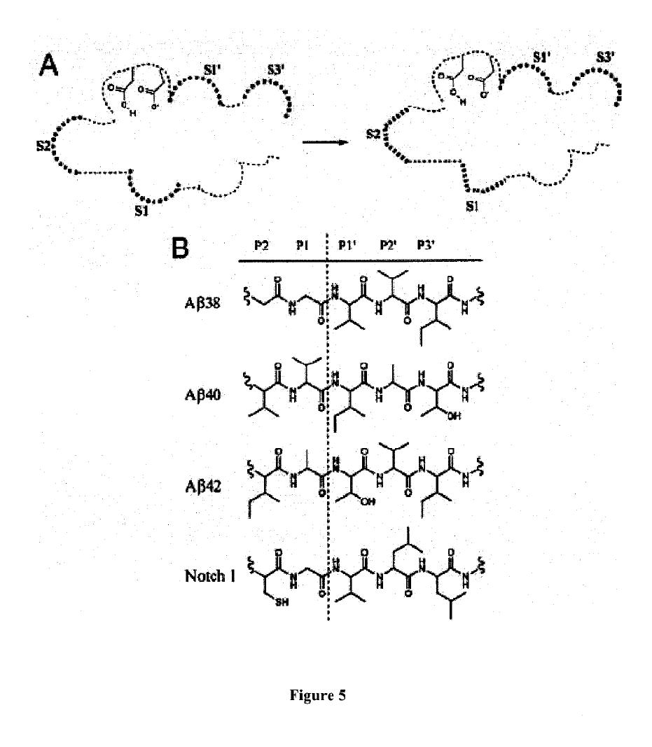

HeLa membrane was incubated with CHAPSO detergent and photoaffinity probe in the presence or absence of excess L458 or CS-1. Labeled presenilin was isolated using streptavidin beads, separated by SDS-PAGE and subsequently western blotted using anti-PS1-NTF antibodies. Again, presenilin is believed to contain the active site of γ-secretase, therefore we examined PS1 photolabeling. We determined that the compounds each labeled PS1-NTF, which migrated at approximately 34 kDa (FIG. 4e). First, as expected, excess L458 at 2 μM completely blocked photoinsertion of each probe. This demonstrated that the active site photolabeling was specific (FIG. 4e). Second, CS-1 up to 100 μM did not block the L505 labeling of PS1-NTF and only slightly inhibited JC-8. This indicated that CS1 binding has no significant effect on the S1′ and S3′ subsites and supports the notion that CS-1 and L458 do not bind at the same site within γ-secretase (FIG. 4e, two upper panels). Third, CS-1 virtually abolished all of the labeling of PS1-NTF by L646 and GY-4 (FIG. 4e, two lower panels), which confirmed that this class of inhibitors directly interacts with γ-secretase and that CS-1 binding alters the S2 and S1 subpockets within the active site. Moreover, CS-2 that is 17-fold less potent than CS-1 for Aβ42 inhibition (FIG. 2) did not alter L505 photolabeling of the S3′ subsite and only partially block GY-4 labeling at 100 μM (FIG. 4f). Clearly, inhibition of the photoinsertion of GY-4 is related to the potency of these AGSI compounds. Lastly, Compound E at 2 μM nonselectively blocked photoinsertion of all four probes (FIG. 4g). Taken together, these results indicate that the binding of CS-1 to an allosteric site in γ-secretase alters the active site architecture, mainly affecting the S2 and S1 (non prime side) subsites (FIG. 5a). It is possible that CS-1-induced conformational changes within the active site of γ-secretase alter the enzymatic interaction with the P2 and P1 residues of Aβ42 (Ile-Ala), yet minimally affect the P2 and P1 side chains of Aβ38, Aβ40, or Notch-1 (Gly-Gly, Val-Val, and Cys-Gly, respectively) (FIG. 5b). Regardless, it is clear that these di-coumarin allosteric γ-secretase inhibitors selectively abolish Aβ42 cleavage over Aβ38, Aβ40, and Notch1 and this selectivity is likely due to alteration within the S2 and S1 pockets of the enzymatic active site.

### Discussion

γ-Secretase cleaves numerous substrates that are involved in diverse biological processes. The multiple substrates of γ-secretase appear to possess little primary sequence homology and consequently, the factors governing cleavage specificity remain unknown. The localization or compartmentalization of γ-secretase substrates has been proposed as one mechanism to control its activity (27-28). In addition to processing multiple proteins, γ-secretase initiates proteolysis of APP at multiple sites. Among the products that result, Aβ42 is more hydrophobic and therefore more prone to aggregate and form the characteristic neurotoxic oligomers and fibrils associated with AD as compared to other β-amyloid species (29). Therefore, factors that promote the generation of Aβ42 are believed to accelerate the pathological cascade leading to AD. Mutations in APP, PS-1, and PS-2 are linked to familial forms of early onset AD (7). The majority of mutations within each of these genes cause an increase in the ratio of Aβ42 to Aβ40 in biochemical, cellular and animal models. Recent studies suggest that alteration of γ-secretase complex dynamics and/or formation of γ-secretase complexes with mutated components can affect the enzymatic cleavage specificity (30-31). Despite these advances in our understanding, little is known regarding the molecular mechanisms that control the specificity of γ-secretase-mediated cleavage at the Aβ40, Aβ42 or Notch1 cleavage locations. Our work has provided the first evidence that changes in the active site architecture can modulate γ-secretase specificity and provides a rationale for the design of selective GSIs targeting the S2 and S1 subsites. Additionally, we present a novel family of small molecule inhibitors that can be used to probe the biology of γ-secretase and may serve as the basis for AD drug development.

First, developing GSIs that preferentially abrogate Aβ42 production over other Aβ species or substrates has been an appealing strategy for AD therapeutics. Establishment of these selective inhibitors could potentially reduce the Notch-related toxicity witnessed with current GSIs and maintain Aβ40 production, which is thought to be neuroprotective against AD (10). In this study, we have identified a coumarin-dimer class of allosteric GSIs (AGSI) that preferentially inhibit γ-secretase-mediated Aβ42 generation over Aβ40, Aβ38, or Notch in vitro as well as in cell-based systems. These AGSIs directly target γ-secretase by binding to an allosteric site within the enzyme, rather than targeting the APP substrate. Furthermore, these coumarin-dimer compounds similarly affect γ-secretase activity for Aβ40 and Aβ38 production and lack the interconnected effect witnessed with the GSMs whereby decreased Aβ42 resulted in increased Aβ38 generation, and vice versa (17). Therefore, these AGSIs represent a class of inhibitors that are distinct from the GSMs (12, 17) as well as previously reported GSIs (14-15, 18). It is noteworthy to point out that coumarin-dimer based compounds have been reported to be active against HIV integrase (32) and human NAD(P)H:quinine oxidoreductase-1 (33), as well as exhibit anticoagulant activity (34). However, the coumarin-dimer compounds that Nolan et al. reported that are most potent against NAD(P)H:quinine oxidoreductase-1 lack the central benzene ring (CS-5) and therefore exhibit a much weaker inhibition of γ-secretase (FIG. 2). Clearly, these compounds possess a distinct structure and activity relationship against NAD(P)H:quinine oxidoreductase-1 as compared to γ-secretase. Therapeutic application of these AGSI compounds needs to be further investigated. Additionally, we have demonstrated that AGSIs bind to an allosteric site within the γ-secretase complex thereby influencing the interaction of γ-secretase with our active-site directed inhibitors. The presented data reveals that AGSI binding is capable of altering the conformation of the catalytic core of γ-secretase within the S2 and S1 subsites. These changes likely are the cause for differential inhibition of Aβ42 over Aβ38, Aβ40 and Notch cleavage by the di-coumarin compounds. Therefore, it is conceivable that other factors influencing γ-secretase cleavage specificity for Aβ42 could similarly affect the S2 and S1 pockets. PS-1 FAD mutations significantly affect Aβ42 production and represent one potential pathological example whereby mutational alteration of the S2 and S1 subsites results in altered enzymatic specificity.

Finally, we have developed a rational method to monitor subtle changes in the conformation of the γ-secretase active site using photoactivatable, active-site directed probes. γ-Secretase is a large multi-protein complex composed of at least four proteins possessing 19 putative transmembrane domains. The complexity of γ-secretase has made acquisition of its crystal structure a formidable challenge and it has not yet been successfully obtained. Our method thereby offers a practical chemical approach for elucidating the action mechanism of inhibitors against the γ-secretase complex and other enzymes in which sufficient resolution of structures are not available or obtainable. These photoreactive compounds are valuable tools for examining the active site of endogenous γ-secretase and can be used to analyze factors that influence its conformation or to investigate differences across varied tissues or cell lines.

In summary, the discovery of these selective AGSIs and development of our multiple photoaffinity small molecule approach has helped to elucidate a mechanism of γ-secretase specificity and shed light on how γ-secretase specificity is modulated. Furthermore, the family of di-coumarin compounds represents a novel class of drug candidates for therapeutic AD development and will be useful probes for unraveling the intricacies of this enigmatic protease under physiological and pathological conditions.

### Materials and Methods

### Reagents, GSIs, and Photoaffinity Probes.

Coumarin-based γ-secretase inhibitors were synthesized in our laboratory and will be published in detail elsewhere while Compound E was synthesized as previously described (35). The syntheses of L458, L646, L505 (3), GY-4 (25), and JC-8 (26) were all previously described elsewhere. The polyclonal anti-NICD-1 SM320 antibody that was produced using a peptide antigen was purified using peptide antigen immobilized resin.

### In Vitro and Cell-Based γ-Secretase Assays.

Cell membranes and solubilized γ-secretase were prepared as described previously (36). The in vitro and cell γ-secretase assays detecting either Aβ38, Aβ40, or Aβ42 cleavage were performed similar as previously described (21, 36). Cleaved product was detected using ruthenylated antibodies that recognize specific APP cleavage sites (Aβ31-38*, G2-10*, or G2-11* antibody for Aβ38, Aβ40, or Aβ42 respectively). The Km and Vmax in the presence and absence of γ-secretase inhibitors were analyzed by non-linear curve fit using the software SigmaPlot 8.0 with the Michaelis-Menten equation (v=Vm [S]/(Km+[S]; v: initial rate; Vm: maximum velocity; Km: the Michaelis-Menten constant, S: substrate).

The in vitro γ-secretase assay detecting Notch cleavage was similar to the assays described above, however there were a few notable differences. First, the substrate used was a directly biotinylated Notch transmembrane domain peptide (Notch1-TM, acetyl-YVAAAAFVLLFFVGCGVLLSRKRRRQHGK-biotin). This Notch substrate was incubated with 40 ng/μl solubilized γ-secretase, 0.25% CHAPSO and 1% DMSO or GSI in the presence of 1×PIPES, pH 7.0 buffer for 2.5 hrs at 37° C. Cleaved product was detected using the affinity polyclonal anti-NICD-1 antibody (SM320), which recognizes the cleaved product and not the substrate, as well as a ruthenylated secondary anti-rabbit antibody. The sample was then similarly incubated with magnetic streptavidin beads and quantified by measuring electrochemiluminescence.

IP-MS Analysis of β-Amyloid Peptides from Cell Media.

Aβ peptide profiles were analyzed by immunoprecipitation/mass spectrometry (37). Aliquots of 1.0 mL conditioned media (DME-HG, Opti-Mem, 10% FBS, Pen/Strep, G418) from N2A mouse neuroblastoma cells overexpressing APP Swedish mutation were immunoprecipitated by monoclonal antibody 4G8 and Protein G+/A agarose beads in the presence of internal standard, Aβ12-28 (10 nM). Aβ peptides were extracted from the beads with α-cyano-4-hydroxycinnamic acid matrix (using as solvent Formic acid/Water/Isopropanol 1:4:4 v/v/v) and spotted on a MALDI target plate prepared by the thin-layer method. The molecular masses of immunoprecipitated Aβ species were measured using a Voyager-DE STR matrix assisted laser desorption ionization time-of-flight mass spectrometer (Applied Biosystems). Each spectrum was collected using 750 laser shots. Mass spectra were calibrated using bovine insulin as internal mass calibrant. Peaks corresponding to Aβ peptides were identified using the measured molecular masses searching against human Aβ peptide.

### Cell-Based Notch Cleavage Assay.

ΔE Notch or empty pcDNA3.1(−) construct was transfected into HEK-293 cells in a 6-well format using Lipofectamine reagent, following manufacturer's instructions. Transfection mixture was incubated with cells for 5 hrs at 37° C. Following incubation, media was removed and fresh media was added back containing 1% DMSO or GSI. This was incubated for 24 hrs at 37° C. after which the cells were washed 1× in phosphate buffered saline and lysed in 1×RIPA buffer (50 mM Tris pH 8.0, 150 mM NaCl, 0.1% (w/v) SDS, 1% (v/v) NP-40, and 0.5% (w/v) deoxycholic acid) containing protease inhibitors. Samples were then centrifuged at 13,000 rpm's at 4° C. and the supernatent was collected and analyzed by Western analysis using either anti-Myc antibody at a 1:1000 dilution or anti-NICD-1 SM320 at a 1:500 dilution.

### AICD Generation Assay and Photolabeling the γ-Secretase Active Site.

The generation of AICD by γ-secretase was performed as previously described (38) using N2A mouse neuroblastoma cells stably overexpressing the APP Swedish mutation (N2A APPsw). Photolabeling experiments are performed as previously described (3).

### REFERENCES

1. De Strooper B (2003) Aph-1, Pen-2, and Nicastrin with Presenilin generate an active gamma-Secretase complex. Neuron 38:9-12.
2. Wolfe M S, et al. (1999) Two transmembrane aspartates in presenilin-1 required for presenilin endoproteolysis and gamma-secretase activity. Nature 398:513-517.
3. Li Y M, et al. (2000) Photoactivated gamma-secretase inhibitors directed to the active site covalently label presenilin 1. Nature 405:689-694.
4. Esler W P, et al. (2000) Transition-state analogue inhibitors of gamma-secretase bind directly to presenilin-1. Nat Cell Biol 2:428-434.
5. Brown M S, Ye J, Rawson R B, & Goldstein J L (2000) Regulated intramembrane proteolysis: a control mechanism conserved from bacteria to humans. Cell 100:391-398.
6. Kopan R & Goate A (2000) A common enzyme connects notch signaling and Alzheimer's disease. Genes Dev 14:2799-2806.
7. Selkoe D J (2001) Alzheimer's disease: genes, proteins, and therapy. Physiol Rev 81:741-766.
8. van Es J H, et al. (2005) Notch/gamma-secretase inhibition turns proliferative cells in intestinal crypts and adenomas into goblet cells. Nature 435:959-963.
9. Deng Y, et al. (2006) Deletion of presenilin 1 hydrophilic loop sequence leads to impaired gamma-secretase activity and exacerbated amyloid pathology. J Neurosci 26:3845-3854.
10. Kim J, et al. (2007) Abeta40 inhibits amyloid deposition in vivo. J Neurosci 27:627-633.
11. Kumar-Singh S, et al. (2006) Mean age-of-onset of familial alzheimer disease caused by presenilin mutations correlates with both increased Abeta42 and decreased Abeta40. Hum Mutat 27:686-695.
12. Weggen S, et al. (2001) A subset of NSAIDs lower amyloidogenic Abeta42 independently of cyclooxygenase activity. Nature 414:212-216.
13. Kukar T L, et al. (2008) Substrate-targeting gamma-secretase modulators. Nature 453:925-929.
14. Mayer S C, et al. (2008) Discovery of begacestat, a Notch-1-sparing gamma-secretase inhibitor for the treatment of Alzheimer's disease. J Med Chem 51:7348-7351.
15. Netzer W J, et al. (2003) Gleevec inhibits beta-amyloid production but not Notch cleavage. Proc Natl Acad Sci USA 100:12444-12449.
16. Borchelt D R, et al. (1996) Familial Alzheimer's disease-linked presenilin 1 variants elevate Abeta1-42/1-40 ratio in vitro and in vivo. Neuron 17:1005-1013.
17. Kukar T, et al. (2005) Diverse compounds mimic Alzheimer disease-causing mutations by augmenting Abeta42 production. Nat Med 11:545-550.
18. Harrison T, Churcher I, & Beher D (2004) gamma-Secretase as a target for drug intervention in Alzheimer's disease. Curr Opin Drug Discov Devel 7:709-719.
19. Schroeter E H, Kisslinger J A, & Kopan R (1998) Notch-1 signalling requires ligand-induced proteolytic release of intracellular domain. Nature 393:382-386.
20. Shearman M S, et al. (2000) L-685,458, an aspartyl protease transition state mimic, is a potent inhibitor of amyloid beta-protein precursor gamma-secretase activity. Biochemistry 39:8698-8704.
21. Yin Y I, et al. (2007) {gamma}-Secretase Substrate Concentration Modulates the Abeta42/Abeta40 Ratio: IMPLICATIONS FOR ALZHEIMER DISEASE. J Biol Chem 282:23639-23644.
22. Tian G, et al. (2002) Linear non-competitive inhibition of solubilized human gamma-secretase by pepstatin A methylester, L685458, sulfonamides, and benzodiazepines. J Biol Chem 277:31499-31505.
23. Osenkowski P, et al. (2009) Cryoelectron microscopy structure of purified gamma-secretase at 12 A resolution. J Mol Biol 385:642-652.
24. Schechter I & Berger A (1967) On the size of the active site in proteases. I. Papain. Biochem Biophys Res Commun 27:157-162.
25. Yang G, et al. (2009) Stereo-controlled synthesis of novel photoreactive gamma-secretase inhibitors. Bioorg Med Chem Lett 19:922-925.
26. Chun J, Yin Y I, Yang G, Tarassishin L, & Li Y M (2004) Stereoselective Synthesis of Photoreactive Peptidomimetic gamma-Secretase Inhibitors. J Org Chem 69:7344-7347.
27. Tarassishin L, Yin Y I, Bassit B, & Li Y M (2004) Processing of Notch and amyloid precursor protein by gamma-secretase is spatially distinct. Proc Natl Acad Sci USA 101:17050-17055.
28. Vetrivel K S, et al. (2005) Spatial segregation of gamma-secretase and substrates in distinct membrane domains. J Biol Chem 280:25892-25900.
29. Jarrett J T, Berger E P, & Lansbury P T, Jr. (1993) The C-terminus of the beta protein is critical in amyloidogenesis. Ann N Y Acad Sci 695:144-148.
30. Placanica L, et al. (2009) Pen2 and Presenilin-1 Modulate the Dynamic Equilibrium of Presenilin-1 and Presenilin-2 {gamma}-Secretase Complexes. J Biol Chem 284:2967-2977.
31. Bentahir M, et al. (2006) Presenilin clinical mutations can affect gamma-secretase activity by different mechanisms. J Neurochem 96:732-742.
32. Zhao H, et al. (1997) Coumarin-based inhibitors of HIV integrase. J Med Chem 40:242-249.
33. Nolan K A, et al. (2007) Coumarin-based inhibitors of human NAD(P)H:quinone oxidoreductase-1. Identification, structure-activity, off-target effects and in vitro human pancreatic cancer toxicity. J Med Chem 50:6316-6325.
34. Zhou H Y, Hong J L, Shu P, Ni Y J, & Qin M J (2009) A new dicoumarin and anticoagulant activity from Viola yedoensis Makino. Fitoterapia 80:283-285.
35. Lammich S, et al. (2002) Presenilin-dependent intramembrane proteolysis of CD44 leads to the liberation of its intracellular domain and the secretion of an Abeta-like peptide. J Biol Chem 277:44754-44759.
36. Li Y M, et al. (2000) Presenilin 1 is linked with gamma-secretase activity in the detergent solubilized state. Proc Natl Acad Sci USA 97:6138-6143.
37. Wang R, Sweeney D, Gandy S E, & Sisodia S S (1996) The profile of soluble amyloid beta protein in cultured cell media. Detection and quantification of amyloid beta protein and variants by immunoprecipitation-mass spectrometry. J Biol Chem 271:31894-31902.
38. Hecimovic S, et al. (2004) Mutations in APP have independent effects on Abeta and CTF gamma generation. Neurobiol Dis 17:205-218.

These results demonstrate that compounds as provided herein are useful for inhibiting Aβ42 secretion from cells.

1-164. (canceled)

165. A compound according to Formula I 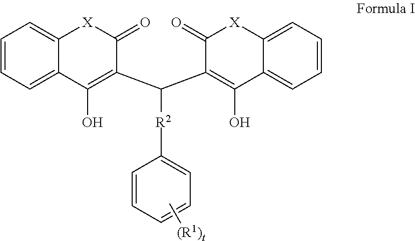
or a pharmaceutically acceptable salt thereof, wherein
each X is O;
each R¹ is independently halo, C₁-C₈ alkoxy, cyano, amino, hydroxy, or C₂-C₈ alkyl;
R² is C₁-C₈ alkylene or C₂-C₈ alkenylene; and
t is an integer from 2 to 5.

166. The compound of claim 165, wherein the compound has the structure: 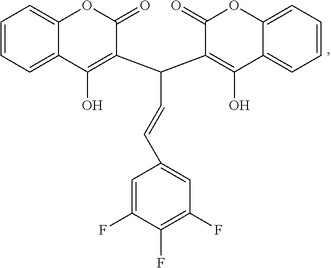 [SMILES: O=c1oc2ccccc2c(O)c1C(/C=C/c1cc(F)c(F)c(F)c1)c1c(O)c2ccccc2oc1=O]
or a pharmaceutically acceptable salt thereof.

167. A composition comprising an effective amount of the compound or pharmaceutically acceptable salt of the compound of claim 165 and a pharmaceutically acceptable carrier or vehicle.

168. A composition comprising an effective amount of the compound or pharmaceutically acceptable salt of the compound of claim 166, and a pharmaceutically acceptable carrier or vehicle.

169. A method for treating or preventing a neurodegenerative disease, comprising administering to a subject in need thereof an effective amount of the compound or pharmaceutically acceptable salt of the compound of claim 165.

170. A method for treating or preventing a neurodegenerative disease, comprising administering to a subject in need thereof an effective amount of the compound or pharmaceutically acceptable salt of the compound of claim 166.

171. The method of claim 169, wherein the neurodegenerative disease is Alzheimer's disease.

172. The method of claim 170, wherein the neurodegenerative disease is Alzheimer's disease.

173. A method for treating or preventing cancer, comprising administering to a subject in need thereof an effective amount of the compound or pharmaceutically acceptable salt of the compound of claim 165.

174. A method for treating or preventing cancer, comprising administering to a subject in need thereof an effective amount of the compound or pharmaceutically acceptable salt of the compound of claim 166.


SEQUENCE LISTING (1 sequence)

SEQ ID NO: 1 - protein, 29 aa, Artificial Sequence
  Description of Artificial Sequence Synthetic peptide
YVAAAAFVLLFFVGCGVLLSRKRRRQHGK